(19) United States
(12) Patent Application Publication
(10) Pub. No.: US 2025/0090842 A1
(43) Pub. Date: Mar. 20, 2025

(54) BIOELECTRIC NEUROMODULATION METHODS AND SYSTEMS FOR NEUROPATHIC PAIN RELIEF

(71) Applicants: THE REGENTS OF THE UNIVERSITY OF CALIFORNIA, Oakland, CA (US); NATIONAL CHENG KUNG UNIVERSITY, Tainan City (TW)

(72) Inventors: Wentai Liu, Los Angeles, CA (US); Meng-Ru Shen, Tainan City (TW); Jung-Shun Lee, Tainan City (TW); Chia-En Wong, Tainan City (TW); Li-Hsien Chen, Tainan City (TW); Ya-Ting Hsu, Tainan City (TW)

(73) Assignees: THE REGENTS OF THE UNIVERSITY OF CALIFORNIA, Oakland, CA (US); NATIONAL CHENG KUNG UNIVERSITY, Tainan City (TW)

(21) Appl. No.: 18/957,670

(22) Filed: Nov. 23, 2024

Related U.S. Application Data
(63) Continuation of application No. PCT/US2023/024482, filed on Jun. 5, 2023.
(60) Provisional application No. 63/349,665, filed on Jun. 7, 2022.

(51) Int. Cl. A61N 1/05; A61N 1/36

(52) U.S. Cl. CPC A61N 1/0551; A61N 1/36071; A61N 1/36139; A61N 1/36196

(57) ABSTRACT

A method and system device to generate and deliver an electric field to a target a peripheral nerve with customized electric fields according to the nerve of interest, which may be superficial or deep in the human body, to elicits action potentials of the target nerve. The system utilizes a stimulator circuit which receives parameters of a stimulation protocol from an external control device, and assembles basic building waveforms, counters, and timers which when combined are directed to a current/voltage driver hardware (CDC) that generates the stimulation protocol.

### CROSS-REFERENCE TO RELATED APPLICATIONS

This application claims priority to, and is a 35 U.S.C. § 111(a) continuation of, PCT international application number PCT/2023/024482 filed on Jun. 5, 2023, incorporated herein by reference in its entirety, which claims priority to, and the benefit of, U.S. provisional patent application Ser. No. 63/349,665 filed on Jun. 7, 2022, incorporated herein by reference in its entirety. Priority is claimed to each of the foregoing applications.

The above-referenced PCT international application was published as PCT International Publication No. WO 2023/239670 A1 on Dec. 14, 2023, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to neuropathic pain relief, and more particularly to electrical peripheral nerve neuromodulation for alleviating painful neuropathic conditions.

### 2. Background Discussion

Neuropathic pain (NP) is characterized by abnormal activation of pain conducting pathways and manifests as mechanical allodynia and thermal hypersensitivity. The management of neuropathic pain remains challenging for clinicians due to ineffective pharmacotherapy. Nerve stimulation at the pain pathway involved central nervous and spinal cord is used for treatment of medically refractory chronic pain in patients who fail to respond to, or are unable, to tolerate pharmacological treatments. However, current viable neuromodulation methods (e.g., Spinal Cord Stimulation (SCS)) require invasive procedures for device implantation. In contrast, sciatic nerve stimulation (SNS) offers advantages of easy access of nerve with non-invasive approach, as a result it becomes a new viable therapeutic solution of benefit to NP.

Accordingly, a need exists for enhanced treatment with non-invasive or minimal invasive electrical neuromodulation methods and systems targeting peripheral nerves such as sciatic nerve. The present disclosure describes a method and system for neuropathic pain relief via peripheral nerve stimulation delivered non-invasively which overcomes many of the adverse events, while providing additional benefits.

### BRIEF SUMMARY

This patent disclosure presents the electrical neuromodulation (bioelectronics medicine) methods and techniques for neuropathic pain relief. Pain has become one of the major challenges of the modern healthcare system. Neuromodulation techniques, such as peripheral nerve stimulation (PNS) and spinal cord stimulation (SCS), have been used to treat debilitating pain in patients who fail to respond to or cannot tolerate pharmacological treatments. Our animal experiment results support that 1 Hz to 100 Hz sciatic nerve stimulation can alleviate both neuropathic pain behaviors and hyperactivation of pain conducting pathways. Neuromodulation techniques can regulate spinal cord neuroinflammation and reduce spinal cord inflammatory protein expression, astrocytic gliosis, and microglia activation. In the present patent disclosure, we disclosed a peripheral electrical neuromodulation system to alleviate painful clinical conditions by targeting the proper peripheral nerves at either upper or lower extremity.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a block diagram for a method of regulating hematopoiesis.

FIG. 2 is an overview of an ANAST system, consisting of four submodules: Graphical User Interface (GUI), Controller (CTL) or Firmware (microcontroller (uCT)), Current/Voltage Driver Circuitry (CDC), and Electrode Array (EA).

FIG. 3 and FIG. 4 is a flow diagram showing the GUI issuing a command to the controller (CTL) or firmware in order to initiate the loading of stimulation parameters from the GUI and setup of the stimulus by CDC to the targeted nerve via the electrode (EA), as shown in FIG. 2.

FIG. 5 is a block diagram of current driver circuitry (CDC) for supporting both anode and cathode current stimulation waveforms.

FIG. 6 is a pictorial showing various example electrode structures (e.g., hook, needle, cuff, surface, and electrode array) which may be utilized.

FIG. 7 is a waveform diagram showing parameters that specify the stimulation waveforms.

FIG. 8 is a data packet diagram for communication between the GUI and firmware of the processor as utilized.

FIG. 9 are waveform diagrams showing basic timing for producing a basic building waveform (BBW) block of the pulse trains defined by N and P counters.

FIG. 10 is a waveform example of a high-resolution controller clock.

FIG. 11 through FIG. 13 are 3D graphs of focusing pattern and associated electrode montage toward achieving the focused stimulation at a specific nerve target using an electrode array (3×6).

FIG. 14 are bar graphs showing that different frequencies of electrical stimulation have distinct impact on modulating the concentration of different types of blood cells within peripheral blood as a consequence of hematopoietic cells mobilizing from bone marrow to peripheral blood.

FIG. 15 is a plot of white blood cell (WBC) concentration relative to a baseline in response to different forms of management.

FIG. 16 and FIG. 17 are bar graphs of treatment results for electrical stimulation (ES) and administered G-CSF shown at day 10.

FIG. 18 is a plot of hemoglobin concentration relative to a baseline for different forms of management as performed in two cycles.

FIG. 19 and FIG. 20 are bar graphs comparing electrical stimulation (ES) and GCSF for alleviating the grading of chemotherapy-related anemia according to results obtained from at least one embodiment of the present disclosure.

FIG. 21 is a plot of platelet concentration relative to a baseline for different forms of management.

FIG. 22 and FIG. 23 are bar graphs comparing chemotherapy augmented with GCSF and with ES according to results.

FIG. 24 is a plot of survival rates showing electrical stimulation reducing hematological adverse events and mortality after chemotherapy.

FIG. 25 are cycle diagrams and bar charts showing electrical stimulation preserving nerve and bone marrow microenvironments.

FIG. 26 are plots of mRNA levels of CXCL12, VCAM1 and SCF within bone marrow depicting how electrical stimulation degrades adhesion molecules within bone marrow and mobilizes stem cells.

FIG. 27 are bar charts for electrical stimulation promoting hematopoietic regeneration.

FIG. 28 is a stimulation protocol and image results from the tests of FIG. 27.

FIG. 29 is a plot comparing chemotherapy performed by itself and chemotherapy augmented with electrical stimulation (ES) showing that ES alters gene expression profile within bone marrow.

FIG. 30 is a stimulation diagram of performing nerve stimulation utilizing an indirect stimulation source exemplified as an ultrasonic source.

FIG. 31 is a pictorial sequence on how electrical stimulation (ES) modulates hematopoiesis and the bone marrow microenvironment.

FIG. 32 is a block diagram of pain classification.

FIG. 33 through FIG. 35 depict neuronal circuits and pathways of pain.

FIG. 36 and FIG. 37 are plots of mechanical tests in animal experiments to validate therapeutic effects of PNS, and stereotactic electric nerve stimulation.

FIG. 38 depicts a stereotactic electrode configuration.

FIG. 39 shows plots indicating thermal and mechanical testing to confirm stimulation parameters.

FIG. 40 are plots 1470 of Plantar Electromyography (EMG) signals recorded to confirm and tune stimulation parameters.

FIG. 41 is a block diagram of a Peripheral Neuromodulation Pain Alleviation (PNPA) System, according to at least one embodiment of the present disclosure.

FIG. 42 is a hierarchical diagram of the PNPA system having a Graphic User Interface (GUI), a microcontroller with firmware (uCT), and Current/voltage Driver Circuit (CDC), according to at least one embodiment of the present disclosure.

FIG. 43 is a flow diagram of waveform control performed on the microcontroller-based PNPA device, according to at least one embodiment of the present disclosure.

FIG. 44 is a block diagram of CDC hardware which generates both cathodic and anodic electric pulses for the stereotactic stimulation electrode arrays according to at least one embodiment of the present disclosure.

FIG. 45 are waveform diagrams of basic building waveforms in constructing waveforms of a treatment protocol, as utilized according to at least one embodiment of the present disclosure.

FIG. 46 is a data packet diagram of an example packet format utilized for communication between the GUI and the PNPA, according to at least one embodiment of the present disclosure.

FIG. 47 are waveform diagram of elements within a set of basic building waveforms (BBW) used by the system in generating the stimulus protocols, as utilized according to at least one embodiment of the present disclosure.

FIG. 48 is a waveform diagram of a balanced symmetric biphasic waveform, generated according to at least one embodiment of the present disclosure.

FIG. 49 are waveforms diagram of four different example stimulation protocols, generated according to at least one embodiment of the present disclosure.

FIG. 50 is a block diagram of a Stereotactic Stimulation Electrode Array (SSEA) configured for delivering electrical stimulation non-invasively, according to at least one embodiment of the present disclosure.

FIG. 51 and FIG. 52 are pictorial views of a multiple ring structure of stereotactic electrode arrays wrapping around an extremity, according to at least one embodiment of the present disclosure.

FIG. 53 is an outline of 5 different tests conducted to evaluate the effect of the neuromodulation in the peripheral nerve using sciatic nerve, utilized according to at least one embodiment of the present disclosure.

FIG. 54 is an experimental procedure summary of evaluating the effect of non-invasive stereotactic neuromodulation using stereotactic stimulation electrode arrays (SSEA) in the peripheral nerve using sciatic nerve which was performed according to at least one embodiment of the present disclosure.

FIG. 55 and FIG. 56 are pictorial views of demonstrating assess points of SSEA for ns-SNS, shown on a human and a rat, according to at least one embodiment of the present disclosure.

FIG. 57 is a pictorial view showing additional placement details for FIG. 56, utilized according to at least one embodiment of the present disclosure.

FIG. 58 is a plot of Plantar EMG signal confirming delivery of nerve stimulation, according to at least one embodiment of the present disclosure.

FIG. 59 are plots of testing results, including a mechanical test of paw withdrawal threshold, a thermal test of paw withdrawal threshold, a mechanical test of cumulative paw withdrawal threshold, and a thermal test of paw withdrawal threshold, utilized according to at least one embodiment of the present disclosure.

FIG. 60 are bar charts showing testing of the expression of the inflammatory proteins, utilized according to at least one embodiment of the present disclosure.

FIG. 61 and FIG. 62 are bar charts of activation of the inflammatory cells in the spinal cord following SNS as measured by immunofluorescent staining, utilized according to at least one embodiment of the present disclosure.

FIG. 63 are plots of pain behavior results in animals receiving NRL+nsSNS and NRL, utilized according to at least one embodiment of the present disclosure.

FIG. 64 and FIG. 65 are bar charts of activation of the inflammatory cells in the spinal cord following SNS as measured by immunofluorescent staining, utilized according to at least one embodiment of the present disclosure.

### DETAILED DESCRIPTION

### 1. Hematopoiesis Modulation System

### 1.1. High Level System Description

The present disclosure is divided into two primary portions. Section 1 through Section 4 pertain to a Hematopoiesis Modulation System which is not claimed in the instant application. Section 5 through Section 8 are directed to a bioelectric neuromodulation apparatus, method and system, for which claims are included in the instant application.

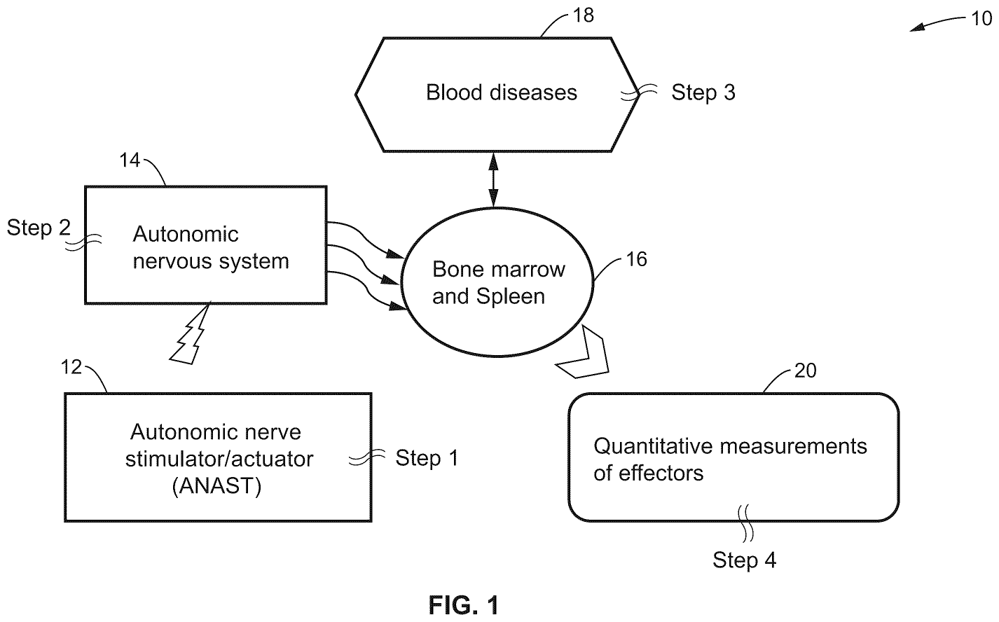

FIG. 1 illustrates an example embodiment 10 of a system and method for regulating hematopoiesis. An Autonomic Nerve Actuator and Stimulator 12 (ANAST) which is coupled (step 1) for acting on the autonomic nervous system 14 and directed at (step 2) the bone marrow and spleen 16 when addressing (step 3) blood diseases 18, as well as for performing quantitative measurement of effectors 20 (step 4).

The method includes the steps of selecting a specific waveform shape based on a system constraint of a waveform generator, and applying a temporal pattern of stimulation to targeted nerves that innervate the bone marrow using the waveform generator, the temporal pattern of stimulation comprising a plurality of single pulse and multiple pulse groups, with constant and randomized inter-pulse intervals between the single pulses and multiple pulse groups, as well as constant or randomized inter-pulse intervals, as well as pulse widths, within the multiple pulse groups themselves.

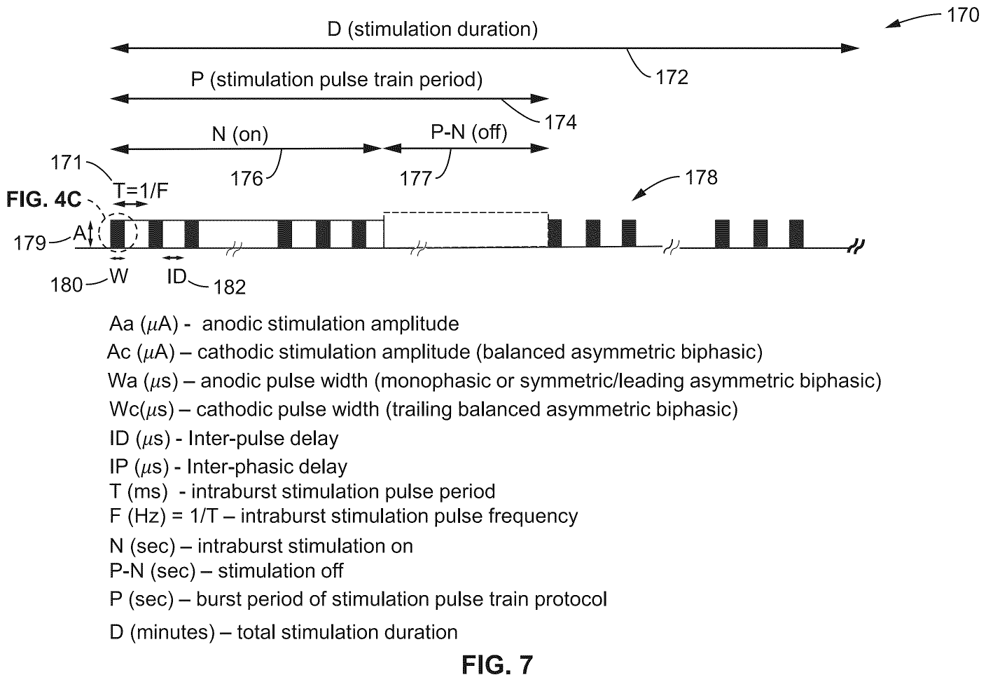

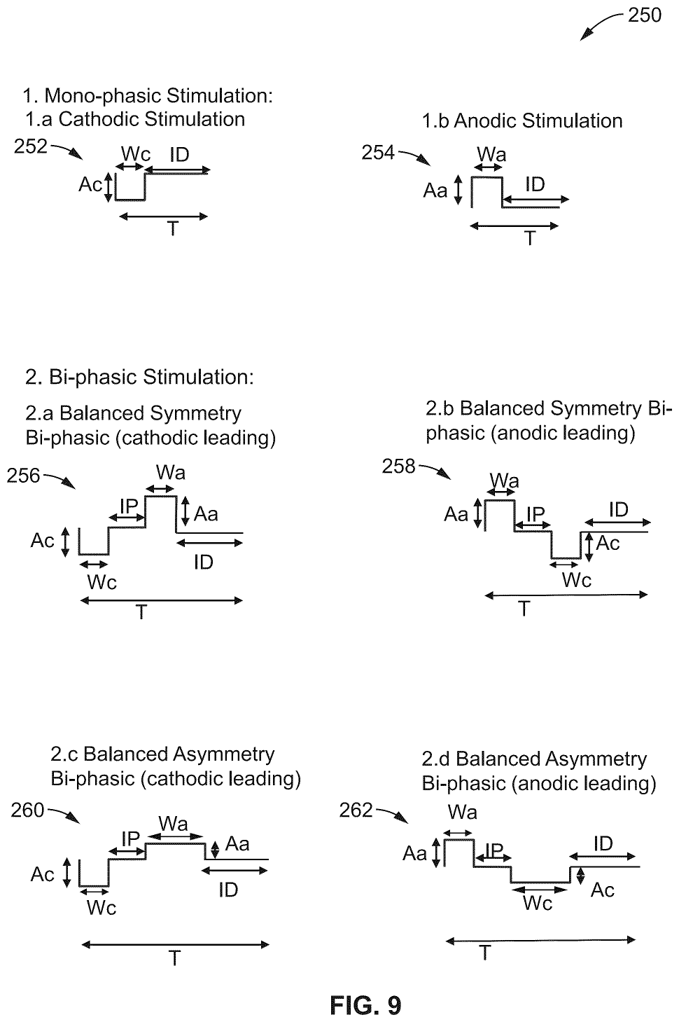

This method and system for bone marrow innervation stimulation may include an electrode(s) to access the targeted nerves either via implantable or transcutaneous mechanism and a stimulus generator operably coupled to the electrode, where the stimulus generator applies electrical stimulation. Connected to at least one electrode, the stimulus generator (ANAST) is configured to transmit to the electrode an electrical signal for innervating (via either sympathetic or parasympathetic nervous system) the bone marrow. A waveform shape of the electrical signal is shown and described in FIG. 7 and FIG. 9. The electrical signal may also utilize a temporal pattern of stimulation, such as comprising a repeating succession of pulse trains, with each pulse train having a plurality of single pulse and multiple pulse groups, with constant or randomized inter-pulse intervals between the single pulses and multiple pulse groups, and also having constant or randomized inter-pulse intervals, as well as randomized pulse width, within the multiple pulse groups themselves. The pulse train repeating in succession innervates and regulates the microenvironments, which closely affects the hematopoiesis at the bone marrow.

### 1.2. ANAST Stimulation System Description

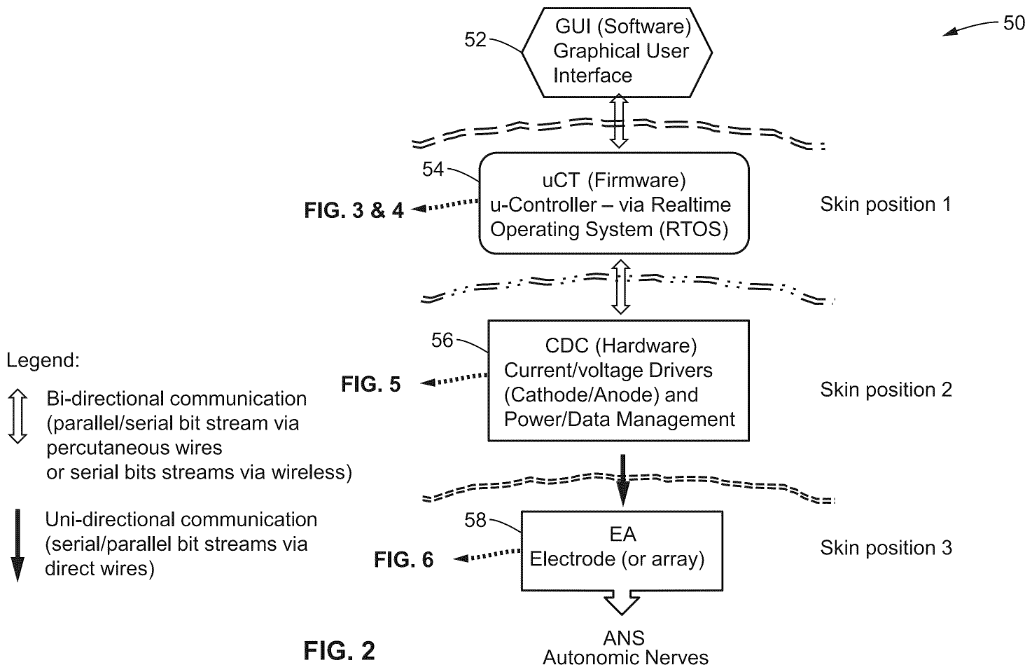

FIG. 2 illustrates an example embodiment 50 of the ANAST system. The ANAST system in this example comprises four submodules: a Graphical User Interface (GUI) 52, Controller (CTL) and/or Firmware (micro-controller (uCT)) 54, Current/Voltage Driver Circuitry (CDC) 56, and Electrode Array (EA) 58. It should also be appreciated that the structures and functions described may be divided in other ways across submodules, which may be more or less than exemplified herein, without departing from the teachings of the present disclosure.

The coupling mechanism between any two of the submodules can be realized either via wired (serial or parallel) or wireless (serial) mode. By way of example and not limitation, a preferred connectivity of bi-directional communications is shown between the upper layers and a uni-directional communications to the lowest level being the electrode(s) or electrode array(s) themselves. The overall ANAST system can be configurated for deployment in supporting the regulation of hematopoiesis through innervating the bone marrow.

The configuration of Skin Position 3, as seen at the bottom of the figure, illustrates that at least one embodiment could house together a subsystem of controller (e.g., processor and firmware), driver, and electrode (array) as an implantable unit and leave the GUI as an external unit. The configuration of Skin Position 2, as seen in the center portion of the figure, shows another embodiment which may house together an implantable unit of the driver and electrode (array), and leave a subsystem of GUI and controller (e.g., processor and firmware) as the external unit. Skin Position 1, shown at the upper portion of the figure, depicts yet another embodiment which may house together the GUI, a control circuit (e.g., processor, memory and firmware), and driver as an external unit and an implantable electrode (array). Thus, the positions of the units in relation to the skin depend on the specific embodiments and its applications.

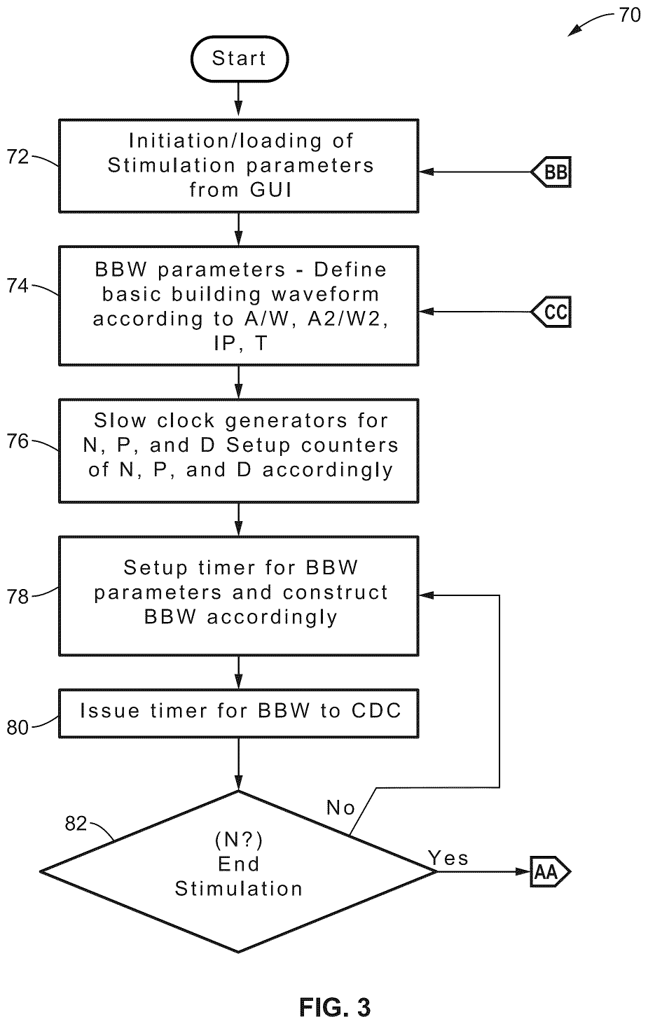

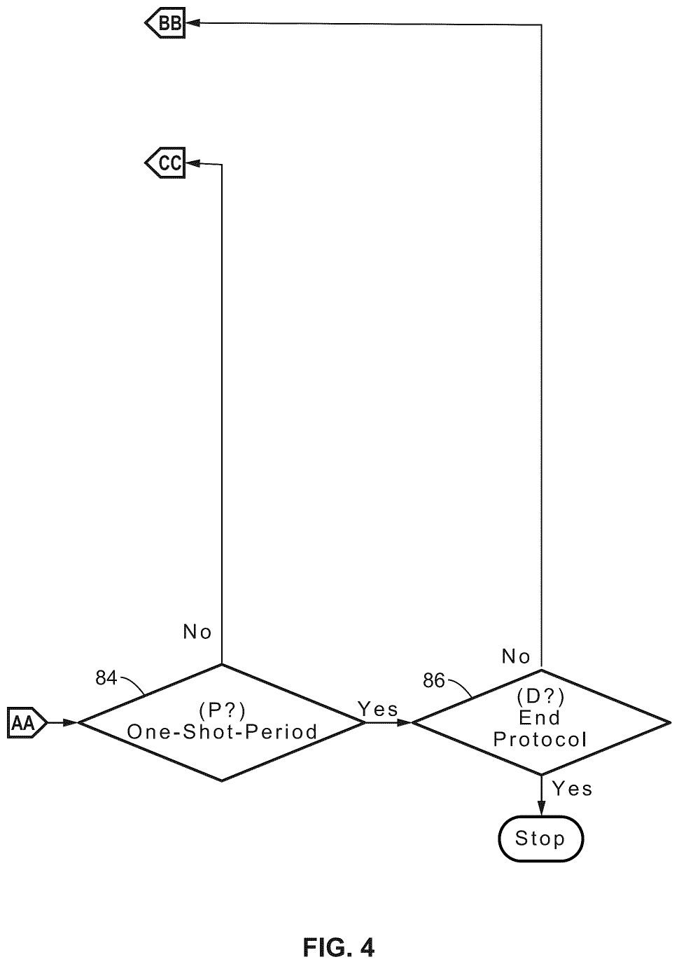

FIG. 3 and FIG. 4 illustrate an example embodiment 70 of a GUI which issues commands to the controller (CTL), such as comprising a processor (e.g., microprocessor with memory and instructions (firmware)) as shown in FIG. 2. It should also be appreciated that other circuit forms may be utilized for generating wave patterns and for controlling operations for initiating the loading of stimulation parameters from the GUI and the setup of stimulation by CDC to the targeted nerve through the electrode(s) (EA), without departing from the teachings of the present disclosure.

In the flow diagram initiation and/or loading 72 of the stimulation parameters is performed from the graphic user interface (GUI). These parameters are converted 74 into Basic Building Waveforms (BBW), described later for FIG. 9, for example according to A/W, A2/W2, IP and T. At block 76 clock generators are activated, such as slow clock generators for parameters N, P and D and setting up counters accordingly. Then at block 78 a timer is set up for the BBW parameters and constructing the BBW. Then execution reaches block 80 which issues time instructions for BBW to the CDC.

A check 82 is performed to determine if the active portion (N) of the stimulation has been completed. If it has not been completed, then execution returns to block 78 with timers being setup again for BBW. Otherwise, if this N phase of stimulation has ended, then execution reaches block 84 in FIG. 4 which determines if the one-shot period (P) has ended.

If the period has ended, then execution returns to block 74 in FIG. 3 where a new period is created. Otherwise, execution reaches block 86 which determines if the entire stimulation protocol has been completed. If it has not been completed, then execution returns to block 72 in FIG. 3 where initialization/loading is performed of the parameters from the GUI; otherwise, this stimulation processing ends.

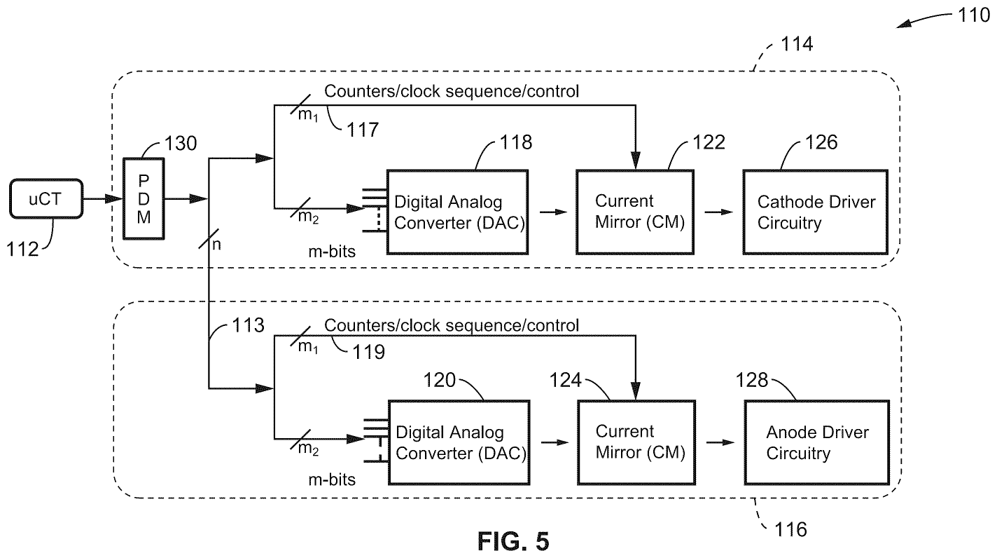

FIG. 5 illustrates an example embodiment 110 of current driver circuitry (CDC), which supports both anode and cathode current stimulation waveforms. A controller 112, such as a microcontroller (processor) with memory and firmware; or other electronic circuit(s) configured for generating sequential strings and controlling stimulation operations is coupled to drivers 114 and 116 through a power/data management (PDM) circuit 130. The controller circuit (herein exemplified as a microcontroller) activates the power/data management unit 130 which provides regulated power to the CDC circuit and associated buffer and clock conditioning/generation. The PDM circuit 130 can be configured for supporting a CDC circuit in either a wired or wireless mode.

It will be appreciated that stimulation requires current levels to be directed to the electrodes; to which the example below is directed. It should, however, be recognized that the stimulation may be regulated based on either current or voltage without departing from the teachings of the present disclosure. Current can be directed through the electrodes toward reaching a given voltage, or directed toward reaching a certain current level. One of ordinary skill in the art will appreciate the interchange between current and voltage when driving a load.

Each driver 114, 116 in this example has a similar structure for driving a stimulation signal at the electrodes. A Digital-to-Analog Converter (DAC) 118, 120, is shown receiving m-bits from the controller. Although these bits are typically sent in parallel, they can be sent as serial information and converted in or before the DAC, without departing from the teachings of the present disclosure.

It should be appreciated that data recovery in a communication sequence can be achieved utilizing either synchronous or asynchronous mode communications. By way of example and not limitation, the embodiment described below utilizes synchronous communications, however, this is not a limitation of the present disclosure which may utilize various communications approaches or protocols for communicating between the controller circuit and current driver circuit.

Following each of the DACs are current mirror circuits 122, 124. The current mirrors are generally utilized here as voltage to current amplifiers, with the proviso described in the previous paragraph. In at least one example embodiment, the current mirrors operate as current amplifiers which have two branches; a reference branch and an output branch, whereby the output current is a multiple of the reference current. The reference current branch is made of N parallel sub-branches such that the overall reference current is equal to the values specified by the N-bit binary code. Each binary bit represents a binary voltage which is converted to current in the drive circuit.

The current mirror could be turned on and off according to the controlled switch (usually connected to the mirror circuitry in serial). This switch is further controlled by the “counter/clock sequence/control) signals provided by the controller circuit (uCT).

The current mirrors output current amplitudes, A_{c} from CM 122, and Aₐ from CM 124, respectively to a cathode driver circuit 126 and anode driver circuit 128. The pulse width of each anode and cathode pulse is specified by parameters W_{k.} and Wₐ, respectively, the resolutions of which are limited by the clock frequency.

It will be appreciated that the generation of these cathode and anode drive waveforms may be accomplished with variations of this circuit, or alternatives, which otherwise are configured for setting signal patterns to drive both cathode and anode circuitry for the stimulation patterns. Accordingly, the present disclosure is not limited to the specific structure exemplified in this figure.

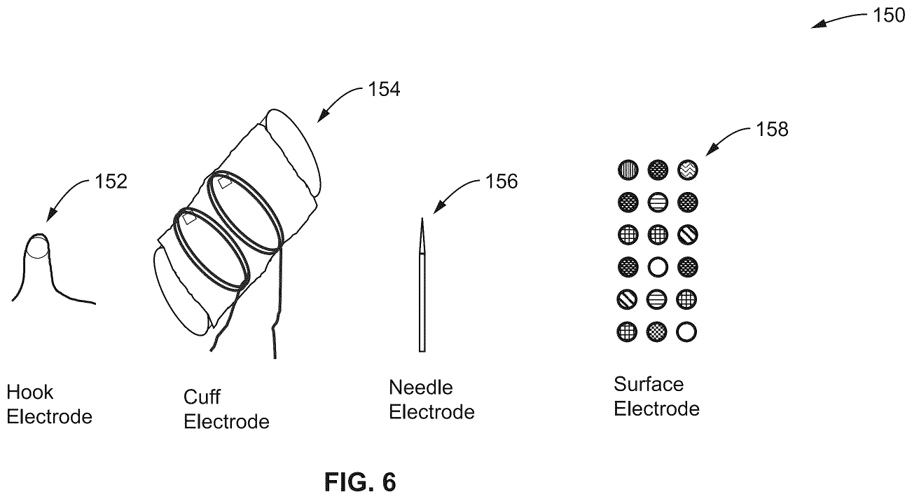

FIG. 6 illustrates an example embodiment 150 of various electrode structures. By way of example, and not limitation, the electrode structures in at least one embodiment can utilize one or more hook electrode 152, cuff electrode 154, needle electrode 156, and surface electrodes 158; while other electrodes known to one of ordinary skill in the art and/or combinations of various electrode types may be utilized without departing from the teachings of the present disclosure. A multiple or a plurality of any electrodes or combinations may be utilized, such as shown in electrode array 158 (comprising surface electrodes). In at least one embodiment at least one electrode array is utilized, which for example may be retained in a fixed or stretchable substrate.

FIG. 7 through FIG. 10 illustrate an example embodiment 170, 210, 250 and 290 of parameters for controlling stimulation.

In FIG. 7 is shown the timing and counters of intraburst stimulation pulse period (T) 171, total stimulation duration (D) 172, burst period of stimulation pulse train protocol (P) 174, intraburst stimulation on (N) 176, value of period that stimulation is off (P-N) 177, which is the idle latency of one-shot period (P) minus the active portion of the stimulation waveform (N), for timing stimulation as set up by the controller to produce the proper waveforms as defined by the parameter set.

The parameters that specify the stimulation waveforms include specification of polarity and mode—LP (leading cathodic or anodic), MO (voltage or current), BP (biphasic), SY (symmetric or asymmetric biphasic); amplitudes, pulse widths, and delay time—Aₐ, Wₐ, A_{c}, W_{c}, IP, ID, T, N, P, and D. One example embodiment is configured with a micro-controller (uCT) and associated memory and firmware for producing the desired stimulus by generating a proper timing sequence for the current driver circuitry (CDC).

A pulse train 178 is shown having amplitude (A) 179, intraburst stimulation pulse period (T) 171, pulse width (W) 180 and inter-pulse delay (ID) 182. The rectangular black sections of the waveform represent the basic building block waveforms as shown in FIG. 9.

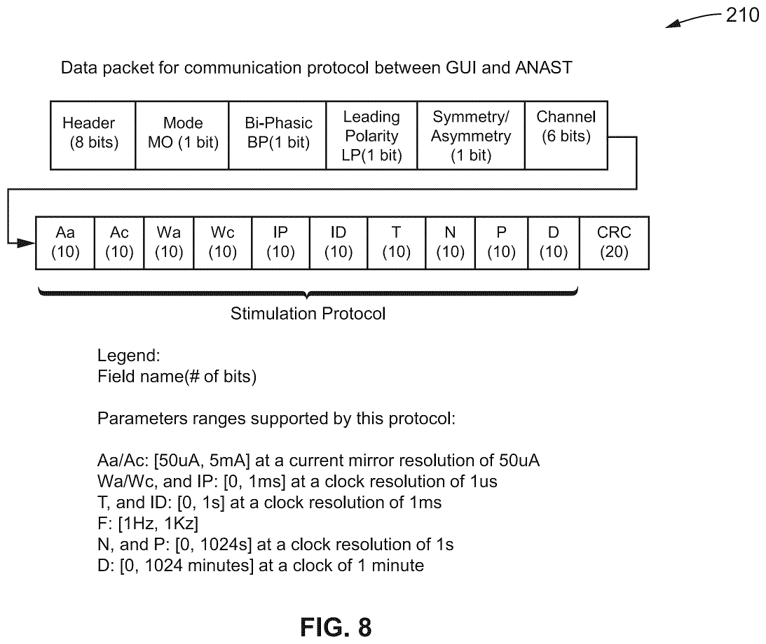

In FIG. 8 is shown a data packet 210 for an example communication protocol between the GUI and a controller and its firmware. The data packet fully specifies the stimulation parameters seen at the bottom of FIG. 7.

The figure also exemplifies a set of counter specifications and its corresponding feasible ranges for the stimulation parameters. The device architecture is able to provide a wide range of parameters for each individual patient subject.

Using the protocol outlined in this FIG. 8, the GUI issues a command to the controller (CTL) in order to initiate the loading of stimulation parameters from the GUI and setup of the stimulus by CDC to the targeted nerve through the electrode or electrode array (EA).

In at least one embodiment a controller circuit (CTL) is exemplified as firmware executing instructions on a micro-controller (uCT) to produce the desired stimulus by generating the proper timing sequence for the CDC accordingly. The timing and counters of T, N, P, and D for controlling the stimulation parameters are set up and controlled by the uCT to produce the proper waveforms defined by the parameter set. The timing is set to produce the basic building waveform (BBW) block of the pulse trains defined by N and P counters. Moreover, it is allowed to change the basic building waveform block of the pulse trains every P periods.

It should be appreciated that the resolution of the parameters in the time domain is limited by the period of the system clock; whereby increasing the frequency of the system clock allows increasing the resolution of the parameters. The counters of T, N, P, and D are updated according to the u-controller clock or corresponding slow clocks derived by uCT. As an example, the resolution of 0.1 μs is achieved for a uCT clock at 10 MHz. The clock generators of the uCT are programmed to produce slow clocks for the counters.

In FIG. 9 are shown examples 252, 254, 256, 258, 260 and 262 of basic timing which can be utilized according to the disclosure for building basic waveform (BBW) blocks of the pulse trains defined by N and P counters.

The first group are configured for generating mono-phasic stimulation as either cathodic stimulation 252, or anodic stimulation 254. In a second group are seen simple bi-phasic stimulation pulses, exemplifying both balanced symmetry (cathodic 256 or anodic 258 leading), and balanced asymmetry (cathodic 260 or anodic 262 leading).

The basic building waveforms (BBW) in FIG. 4C can be realized by various control circuits, for example a microcontroller containing firmware, or by hardware such as System-on-Chip (SoC), or Application-Specific Integrated Circuit (ASIC), or utilizing other forms of sequencing circuitry or combinations thereof. A pulse train (PT) is composed of a series of N (Counter N) basic building waveforms (BBWs). The One-Shot-Protocol (OSP) is in turn composed of a PT and followed by an idle latency of P-N(Counter P). The One-Shot-Protocol (OSP) is repeatedly generated until the counter D has expired.

Moreover, the controller circuit is allowed to change the basic building waveform block of the pulse trains every P period. It will be noted that the resolution of the parameters in the time domain is limited by the system clock frequency. The counters of T, N, P, and D are updated according to the controller circuit clock or corresponding slow clocks derived by the controller circuit (e.g., processor, microcontroller, SoC, ASIC, and/or other circuitry configured for pulse generation).

Each electrode can be programmed as cathode or anode polarity in a bipolar configuration mode or as cathode or anode in monopolar mode. Furthermore, it is feasible to further support symmetric and asymmetric bi-phasic waveforms with interphasic delay (IP) in either bipolar and/or monopolar configurations.

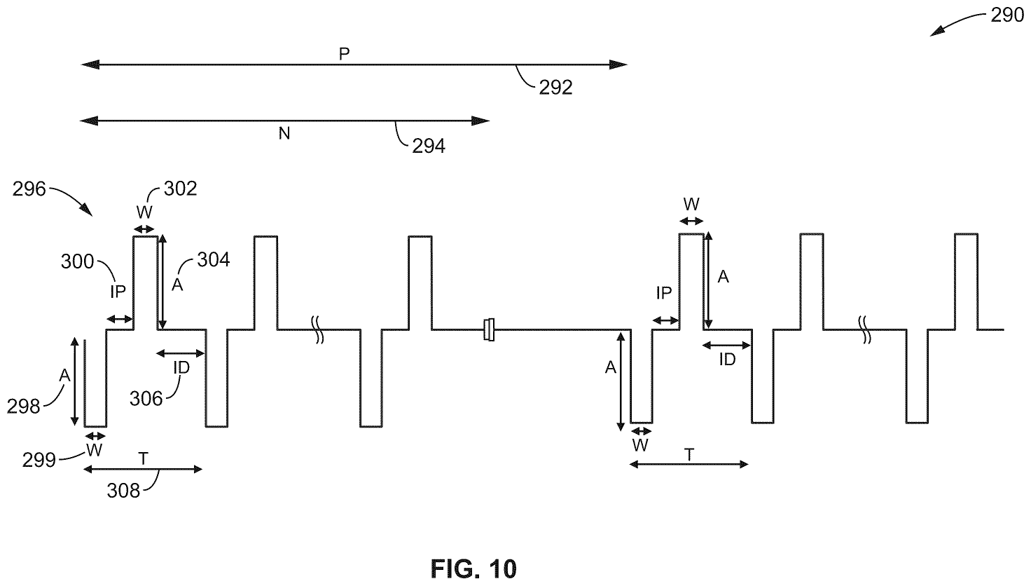

In FIG. 10 is seen an example 290 of a stimulation waveform, exemplifying a one-shot protocol (cathodic leading). By way of illustrative example, and not limitation, a resolution of 0.1 μs of waveform 296 is achieved for an exemplified microcontroller clock at 10 MHz. This figure demonstrates implementation of the stimulation protocol of a balanced symmetric bi-phasic waveform (P) 292 with 2 seconds on (N) 294, 8 seconds off (P-N), and then 60 minutes (D) with BBW at 20 Hz (F=1/T) of balanced symmetric biphasic waveforms with the pulse width of 0.2 ms (W), pulse amplitude of 0.5 mA (A) 298 and 304, a 0.1 ms (IP) 300, cathodic width 299 (W_{c}), anodic width 302 (Wₐ), and T 308 exemplified as being 50 ms. This waveform can be realized using 1 s resolution of the clock for W, IP; a clock of 1 ms for T; a slow clock at 1 s for N, P; and a slow clock at 1 minute for D.

The electrical signal may also be composed of a temporal pattern of stimulation comprising a repeating succession of pulse trains (e.g., the right side of FIG. 10 showing a second pulse train) each pulse train comprising a plurality of single pulse and multiple pulse groups, with constant or randomized inter-pulse intervals between the single pulses and multiple pulse groups, as well as constant or randomized inter-pulse intervals within the multiple pulse groups themselves, the pulse train repeating in succession to innervate and regulate the microenvironments, which closely affect the hematopoiesis at the bone marrow.

Each electrode can be programmed as cathode or anode polarity in bipolar configuration or as cathode or anode in monopolar mode. Furthermore, it is feasible to further support symmetric and asymmetric biphasic waveform with interphasic delay (IP) in either bipolar and/or monopolar configurations.

The current driver circuitry (CDC) seen in FIG. 5 supports both anode and cathode current stimulation waveforms as shown in FIG. 9. By way of example and not limitation, each driver is composed of a Digital-to-Analog Converter (DAC) and a current mirror (CM) such that the output current amplitude, Aₐ or A_{c}, is induced, respectively. The pulse width of each anode and cathode pulse is specified by Wₐ and W_{c}, respectively, whose resolution is limited by the clock frequency.

The ranges for utilizing the ANAST system to augment a chemotherapy treatment are generally according to the following parameters and ranges:
(a) Frequency: about 2 Hz to about 100 Hz.
(b) Waveform inter-phasic delay: about 0 to about 1 ms.
(c) Duration of each phasic pulse: about 0.05 ms to about 3 ms.
(d) Pulse train: stimulation on (about 1 to about 5 seconds) at about 2 Hz to about 100 Hz and stimulation off (about 1 to about 10 seconds). For example, about 1 second on at about 50 Hz and about 9 seconds off.
(f) Amplitude: about 0.05 mA to about 200 mA.
(g) Simulation duration: about 1 minute to about 90 minutes with a repeating pattern defined by “Pulse train”. For example, about 1 second on at about 20 Hz and about 9 seconds off for 60 minutes (total pulses: 20 pulses×60 secs/min×60 mins/10 secs=20×360 pulses=7,200 pulses for 60 minutes).
(h) Natural Biomimetic waveforms which mimic biological signals that represent the firing sequences and oscillation patterns by a neuron or a cluster system of neurons. Examples include EMG, EEG, sympathetic tones, parasympathetic tones, and similar neural activity.
(i) Synthetic Biomimetic waveforms with randomized interphasic delay, pulse width, and amplitude at either a Poisson or Gaussian distribution.

### 1.3. Promoting Hematopoiesis by Electrical Stimulation

Electrical Stimulation (ES) targets the sympathetic nerve innervating bone marrow toward priming its microenvironments after chemotherapy. The results from testing performed in the present disclosure have demonstrated that electrical stimulation of sciatic nerve rescues bone marrow microenvironment from chemotherapy-induced injury, consequently reducing hematologic toxicity and thus mortality.

The therapeutic stimulation provided according to the present disclosure can access (stimulate) the nerves either by an invasive or non-invasive stimulation. Invasive delivery involves the use of direct electrical stimulation to an electrode/electrode array. In non-invasive stimulation the electrical stimulation is created through an indirect mechanism. In at least one embodiment, a form of ultrasound neuromodulation may be utilized in which the ultrasonic particle motions at the nerve are converted into a stimulation force (e.g., electrical stimulation). For example, as these tissues are conductive, particle motion created by an ultrasonic wave induces an electric current density generated by Lorentz forces. This can be enhanced in some cases with magnetic fields generated to pass through the nerve tissue to accentuate the stimulus.

The electrical stimulation described herein is equally applicable to both direct and indirect stimulation of the nerves. By way of example the electrodes/electrode array seen in FIG. 6 can be replaced with indirect operating electrodes, such as in the form of ultrasonic emitters with the cathode and anode drivers in FIG. 5 incorporating an ultrasonic oscillator, or otherwise receiving an ultrasonic oscillation signal.

It should also be appreciated that testing was performed at the sciatic nerve in the test results for the sake of simplicity of illustration, as the sciatic nerve notch is readily accessible for stimulation. However, it will be recognized that the described stimulation would have similar effect on other locations in the nervous system, as the nerve fibers have similar structures and neural activation potentials.

It should be appreciated that bone marrow is innervated by both sympathetic nervous system that is emerged from thoracolumbar spinal cord section and parasympathetic nervous system emerging from cranial nerves and sacral spinal cord section. Thus stimulation, at locations or regions of nerve fibers along both nervous systems mentioned above, will eventually reach the bone marrow and is able to regulate hematopoiesis.

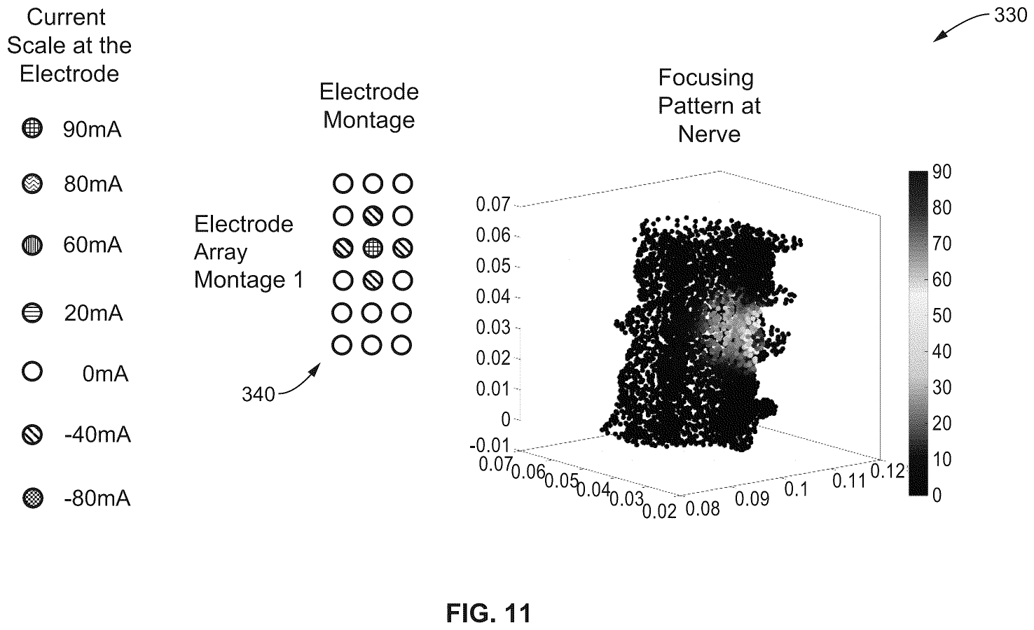

FIG. 11 through FIG. 13 illustrates an example embodiment 330, 350 and 370 of electrode array montages 340, 360 and 380 for achieving a focused stimulation at a specific nerve target, such as a 3×6 electrode array, given by example and not limitation as arrays of various x and y dimensions may be utilized in the present disclosure without limitation.

In these figures, the electrode array montage may comprise either one operating directly or indirectly; for example, the direct stimulation of electrical stimulation through each electrode of the array, or generating an indirect stimulation signal (e.g., ultrasound) which is converted at the nerve it is focused upon into a stimulation. In either case, the desired nerve fiber region can be targeted by (direct or indirect) electrical stimulation if a proper current montage from the array is utilized. Accordingly, the use of focused ultrasound (US) can reach the desired depth of nerve fibers at a pre-defined focality by properly selecting parameters, such as intensity, frequency, acoustic pressure, burst cycle, pulse rate, and duty cycle, and other US related parameters when activating sympathetic nerves.

Each figure depicts a current scale (e.g., from −80 mA up through +90 mA) on the left for interpreting the electrode states in the montage, with the right of each figure depicting a 3D focusing pattern with a scale in meters at the tissue (nerve embedded). By way of example and not limitation, each electrode (or transducer) in the array may be approximately 1 cm diameter with a 3 cm pitch. These features may be scaled down by an order of magnitude, such as in a larger array, or scaled up by a factor of up to four, with relative pitch being determined by the specific implementation and application.

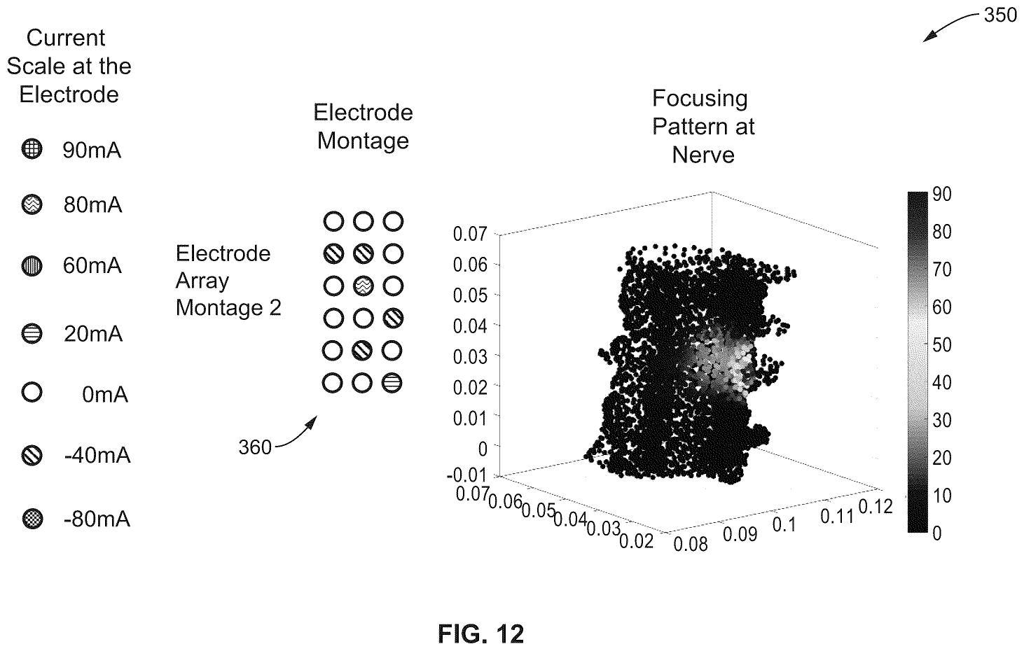

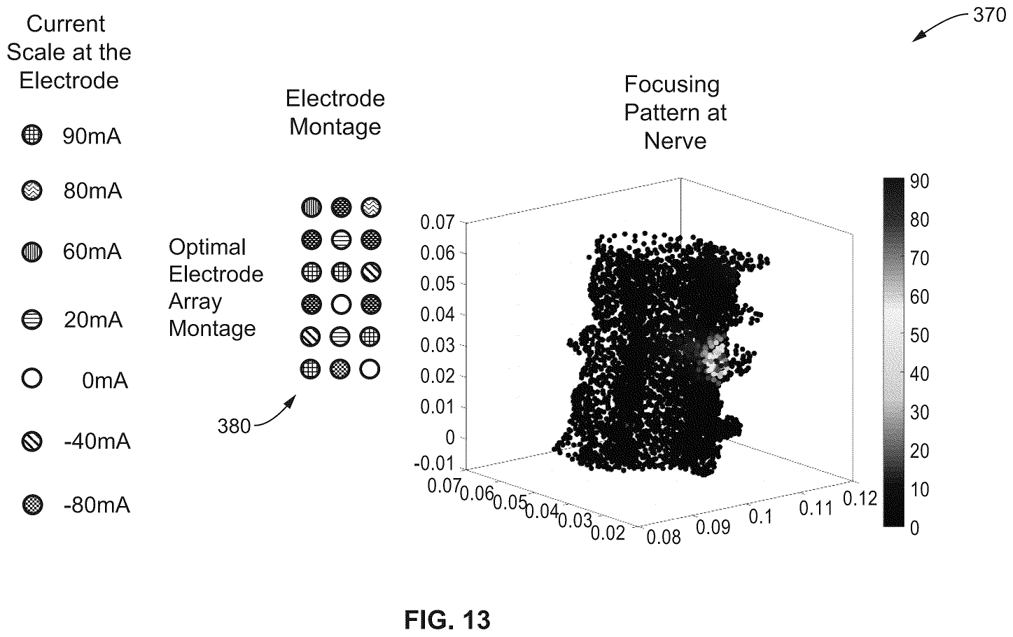

In FIG. 11 and FIG. 12 is seen a first and second electrode array montage, while FIG. 13 depicts an optimal array montage. In at least one embodiment, “optimal” is defined in this context in the sense of electrical field intensity (m/V²) and the focality measurement (cm) of the electrical field at the desired stimulation target.

1.4. Electrical Stimulation of Autonomic Nerve Accelerates Recovery from Neutropenia and Thrombocytopenia Induced by Chemotherapy

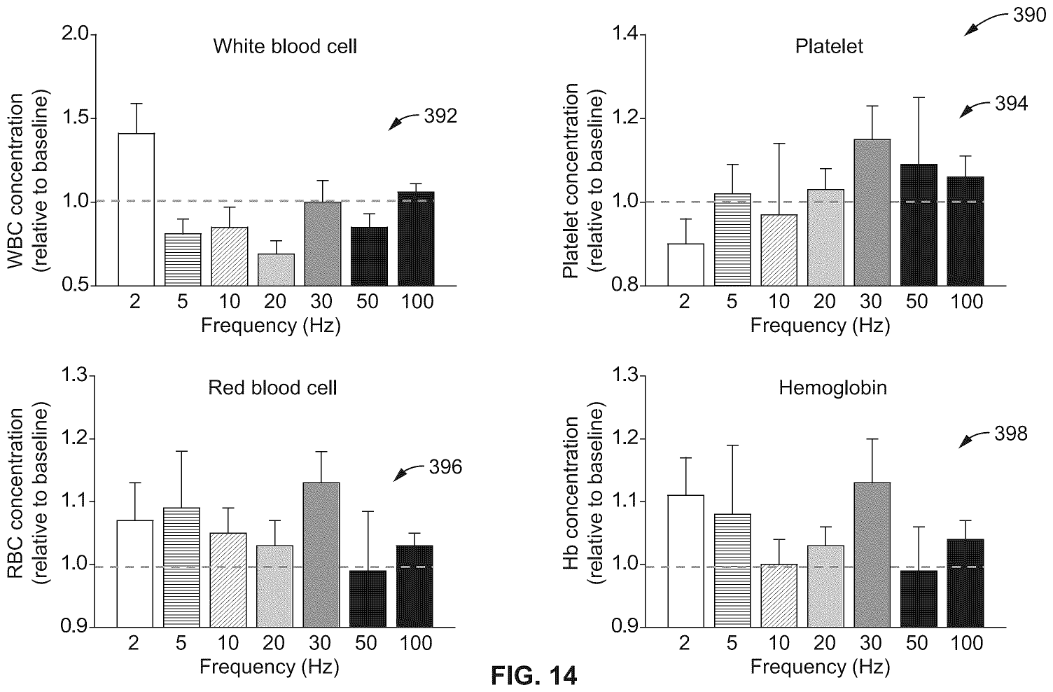

FIG. 14 illustrates an example embodiment 390 of electrical stimulation of the autonomic nerve for modulating peripheral blood cells. The electrical stimulation was performed using different frequencies applied to the nerve (e.g., sciatic nerve in this example test) of SD (Sprague-Dawley) rats for a period of time (e.g., 60 minutes), and then blood samples were obtained for performing a complete blood count. The figure illustrates that different frequencies of electrical stimulation have distinct impact on modulating the concentration of different types of blood cells within peripheral blood, which is the consequence of hematopoietic cells mobilizing from bone marrow to peripheral blood.

In the present disclosure other ranges have been tested for the rat experiments, including the use of pulse widths in the range from 0.05 to 3 mS and current amplitudes from 0.25 to 3 mA. For human subjects the current amplitude range is set from 0.05 to 200 mA.

The figure depicts bar charts for white blood cell concentration 392, platelet concentration 394, red blood cell concentration 396 and hemoglobin concentration 398, at frequencies from 2 Hz to 100 Hz. It can be seen from these charts that these concentration levels can be significantly altered depending on the frequency of stimulation utilized. Thus, the parameters can be modulated, such as frequency in this case, by ANAST toward optimizing the tradeoffs between different physiological characteristics, such as concentration of white blood cells, red blood cells and hemoglobin.

### 2. Hematologic Adverse Events

The use of electrical stimulation according to the present disclosure is applicable to a wide range of chemotherapeutic agents. For the sake of simplicity of illustration, the testing performed is primarily directed to one such agent, “carboplatin”, however, the method and apparatus of the present disclosure is not limited to this one chemotherapy agent.

Chemotherapy-induced hematological toxicity includes damage of hematopoietic stem cells and nerve injury within bone marrow microenvironment. Some chemotherapy agents result in nerve damage such as platinum drugs, taxanes, vinca alkaloids, proteasome inhibitors, and alkylating agent, which in turn disrupts the hematopoiesis by deteriorating the innervation of bone marrow via adrenergic, cholinergic, and peptide receptors. Involving with cytokines and chemokines, the disrupted cascade pathways of molecular signaling prevent the normal function of both endosteal and vascular niches, a critical organism for hematopoiesis-differentiation, proliferation, and migration of Hematopoiesis Stem Cells (HSC). Specifically, damage at both endosteal and vascular niches in bone marrow exacerbates the innervation mechanism via neuroreceptors of Beta-2, Beta-3, Apha-1, Alpha-2. Sympathetic nerve mainly innervates bone marrow by regulating these receptors and trickling down the regulation of molecular pathway signaling, critically the adhesion molecular—CXCL12 (cytokine), and CXCR4 (chemokine). ES applied at the sympathetic nerve has been shown in the present disclosure to provide a high degree of success in preserving the nerve and activating the neuroreceptors and down regulating the critical cytokines and chemokines. Accordingly, the present apparatus and method significantly facilitates hematopoiesis.

As a reference, G-CSF (GranuloCyte Stimulation Factor—a cytokine) has been commonly applied after treatment of various chemotherapy agents. G-CSF activates its own molecular signaling pathways, such as down regulation of CXCL12 in order to facilitate hematopoiesis—differentiation, proliferation, and migration. It should be appreciated that the studies in this present disclosure show that the application of ES outperforms the use of G-CSF in chemotherapy treatments.

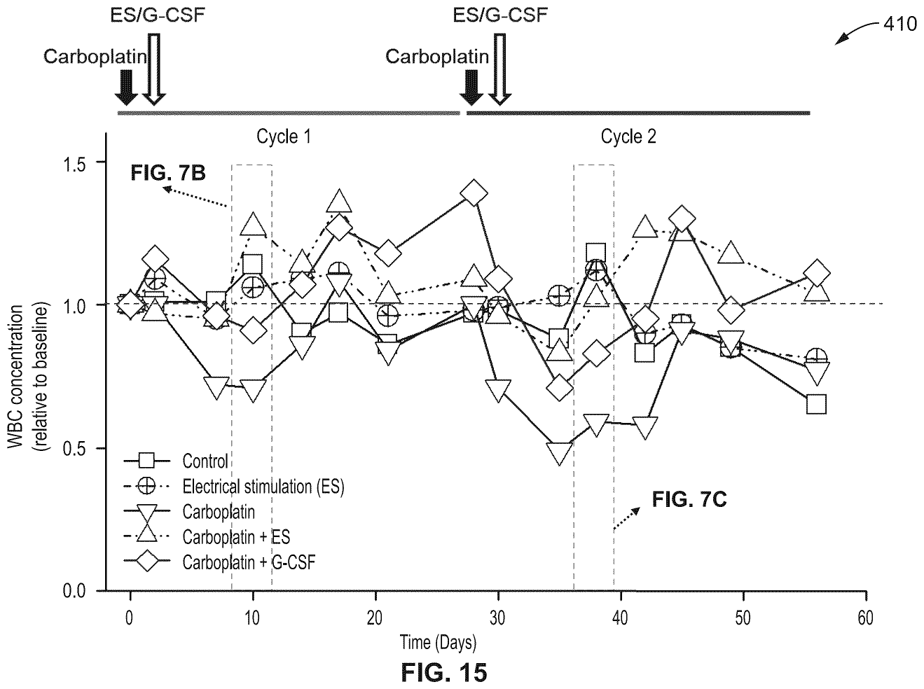

FIG. 15 through FIG. 17 illustrates an example 410, 430, 450 of results from evaluating whether ES can reduce chemotherapy agent-related hematological adverse effect. By way of example and not limitation, the specific chemotherapy agent utilized in this test was carboplatin.

In FIG. 15 is seen white blood cell concentration for each of the five groups of rats which were tested over two cycles of testing. Male SD rats (weighing 350 to 400 g) were used for studying chemotherapy-induced cytopenia. The rats were divided into five experimental groups: control group; electrical stimulation group; carboplatin group; carboplatin+electrical stimulation (ES) group; and carboplatin+G-CSF group.

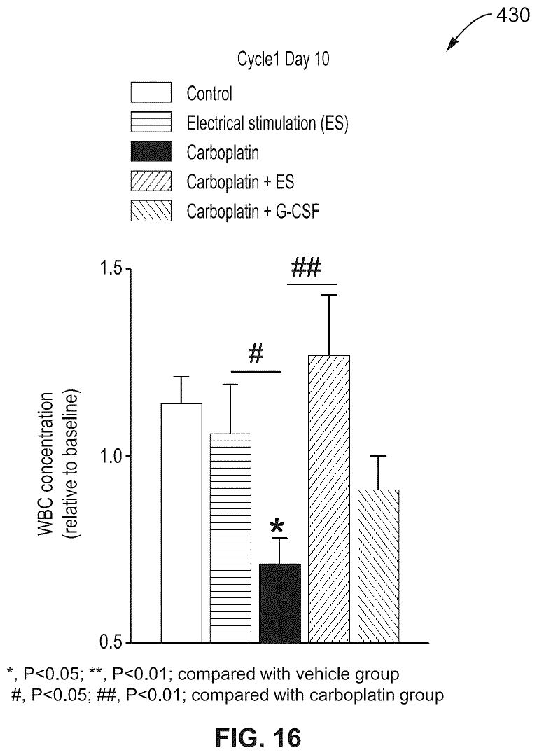

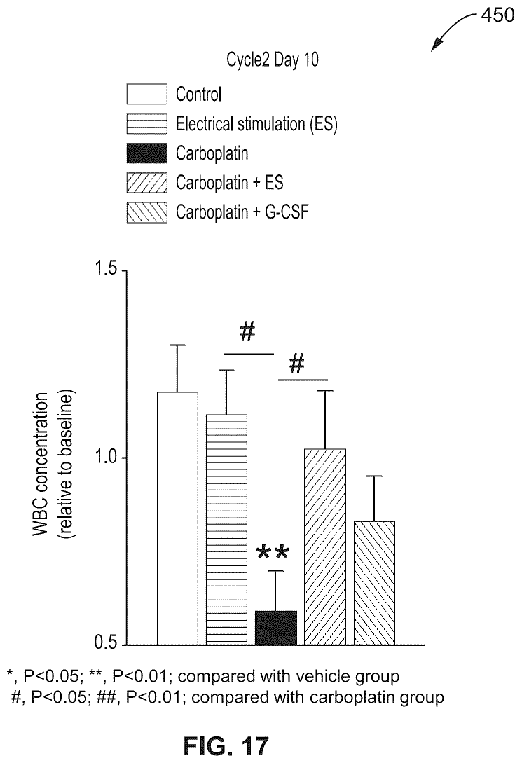

In FIG. 16 and FIG. 17 are shown bar graphs of the results for each group in day 10 of the first and second cycle, respectively. The P-value for statistical analysis (P) is noted in each of these figures.

For each cycle of treatment, a single dose of carboplatin (e.g., 60 mg/kg) or vehicle (saline) was injected intraperitoneally on day 0. On day 2, electrical autonomic nerve stimulation was performed for a specified period (e.g., 60 minutes), or a single dose of G-CSF was administered on the rats according to the different experimental groups. In the example treatment each cycle of treatment is considered to be 28 days, however, a treatment cycle could span from one to eight weeks.

In at least one embodiment the nerve stimulation can be generated at frequencies from 1-100 Hz, with a current level from approximately 0.05 mA to 200 mA, using a balanced symmetric and asymmetric biphasic waveform. In at least one preferred embodiment, the frequency was approximately 20 Hz at a current level of approximately 0.5 mA and an intraburst stimulation period N of two seconds, and a burst period of approximately P=10 seconds.

Blood samples for complete blood count (CBC) were collected in EDTA tubes on various days (day 0, 2, 7, 10, 14, 17, 21, and 28).

Compared to the rats in the carboplatin group, the severity of carboplatin-induced neutropenia was significantly alleviated in the group receiving carboplatin and electrical stimulation according to the present disclosure.

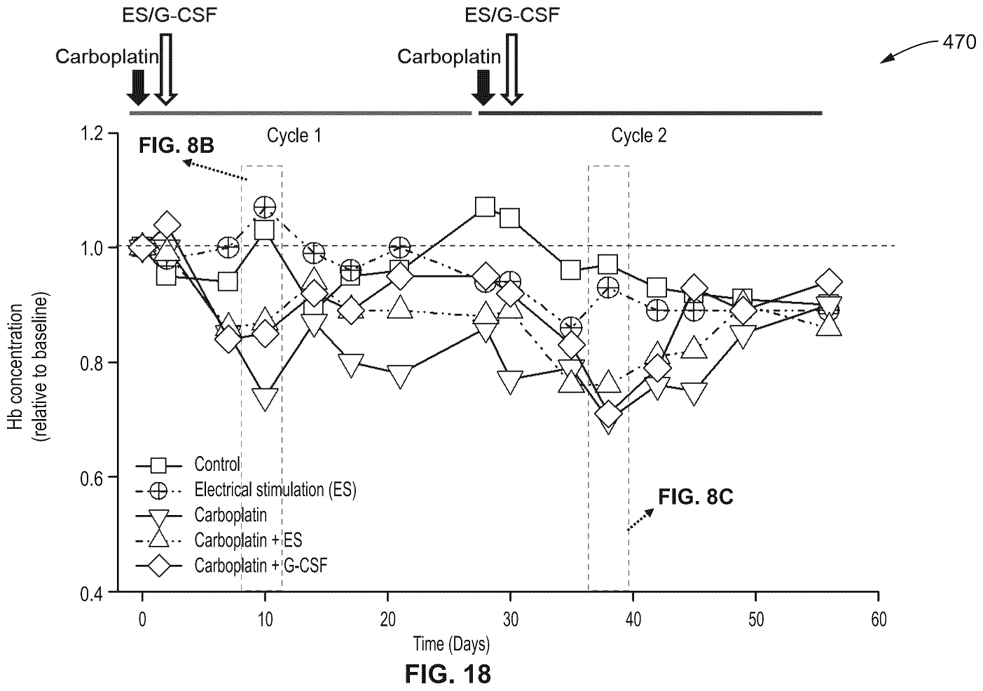

FIG. 18 through FIG. 20 illustrate an example 470, 490, 510 of results obtained from evaluating hemoglobin levels during testing of the present disclosure at each time point from the rats from the same five experimental groups.

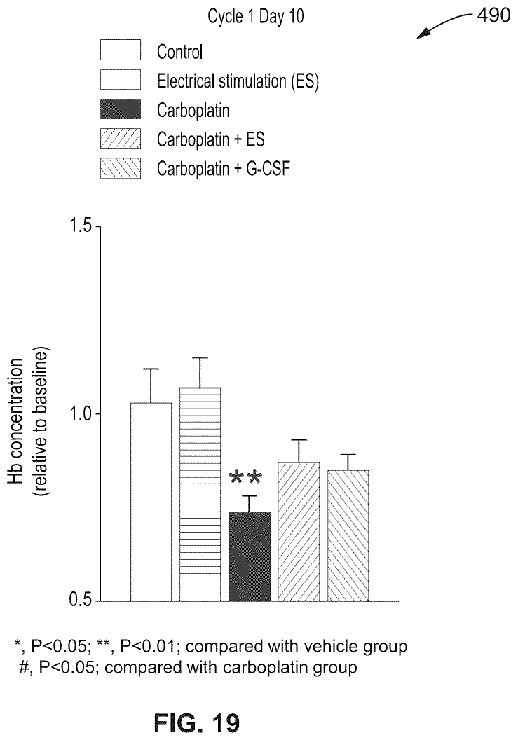

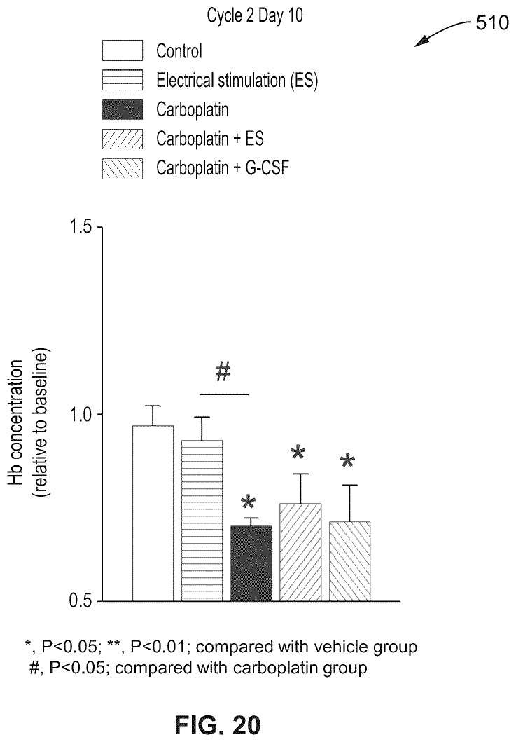

In FIG. 18 is shown plots for each of the five groups of rats which were tested over two cycles of testing. In FIG. 19 and FIG. 20 bar graphs of the results for each group in day 10 of the first and second cycle are shown, respectively. The P-value for statistical analysis (P) is noted in each of these figures.

Whether rescue by electrical stimulation or G-CSF, the concentration of hemoglobin was both significantly decreased after carboplatin administration. It can be seen that the ES group has increased concentrations in relation to the control group, while performing ES with the carboplatin provided a notable increase in Hb concentration.

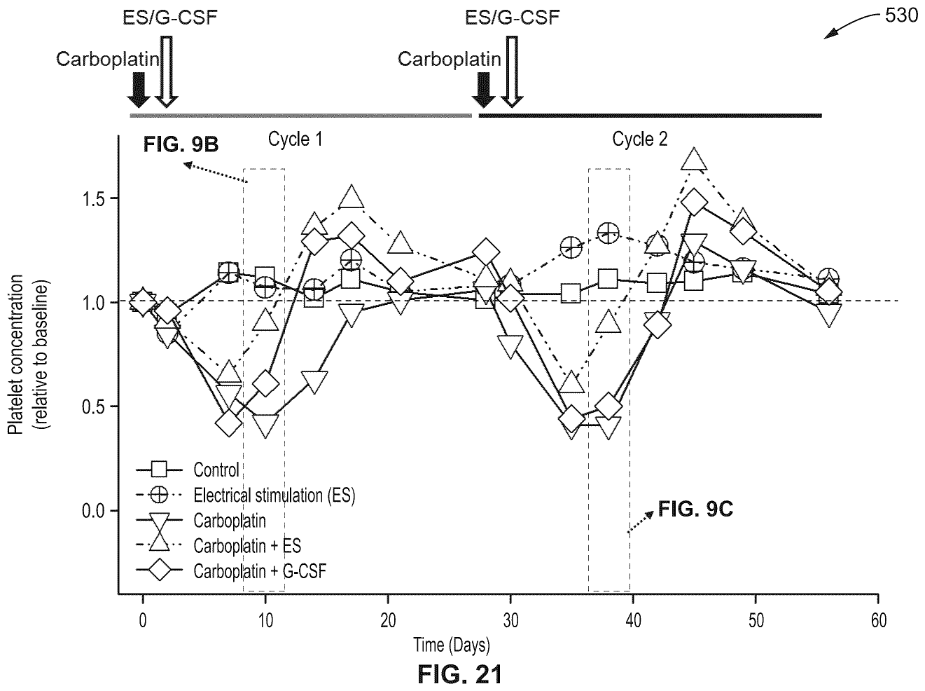

FIG. 21 through FIG. 23 illustrate an example 530, 550, 570 of results from evaluating platelet concentration at each time point from the rats in the same five experimental groups as described above.

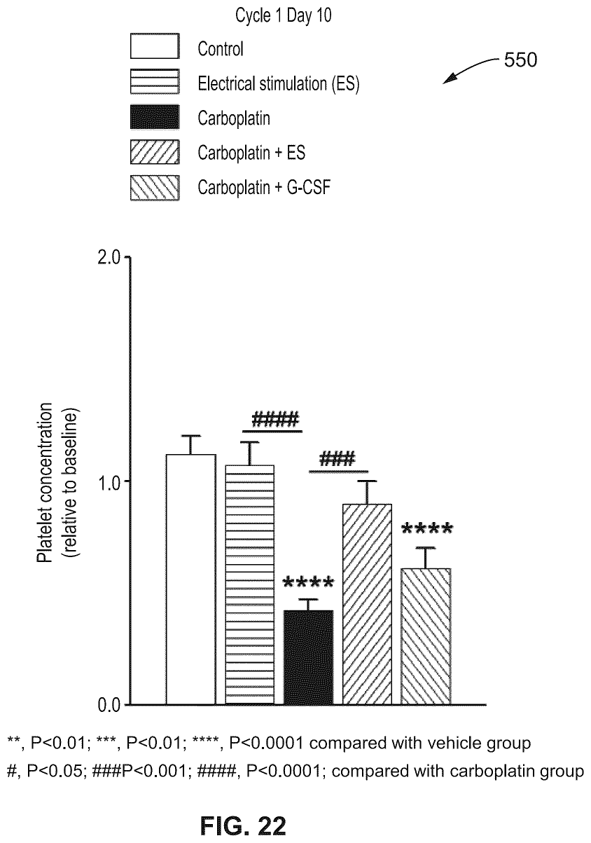

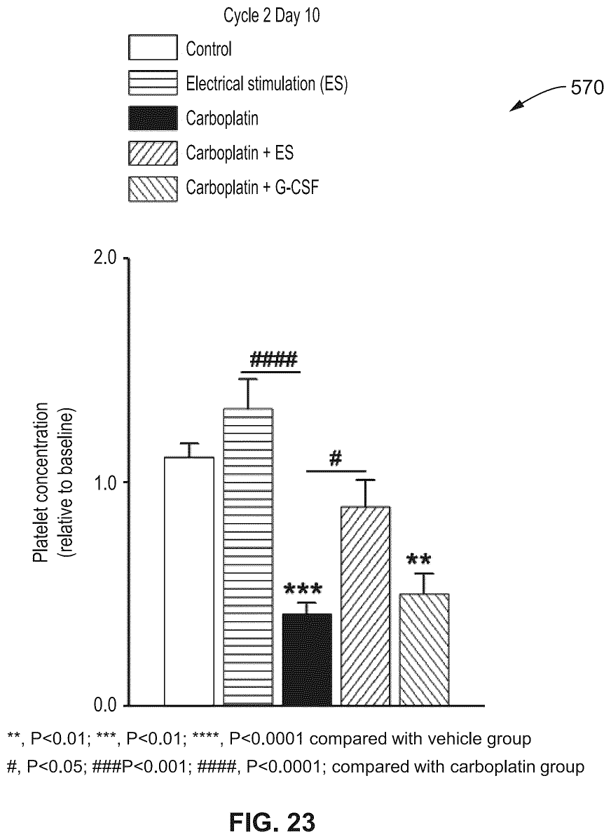

In FIG. 21 is shown plots for each of the five groups of rats which were tested over two cycles of testing. In FIG. 22 and FIG. 23 are bar graphs of the results for each group in day 10 of the first and second cycle, respectively. The P-value for statistical analysis (P) is noted in each of these figures. It can be seen in these bar charts that compared to the control group, the platelet counts were significantly reduced in the carboplatin and carboplatin+G-CSF groups. In contrast, there is markedly higher platelet count in the carboplatin +ES group. The data demonstrates that ES alleviates the adverse effect of thrombocytopenia caused by carboplatin.

2.1. Electrical Stimulation Reduces Hematological Adverse Events and Mortality after Chemotherapy

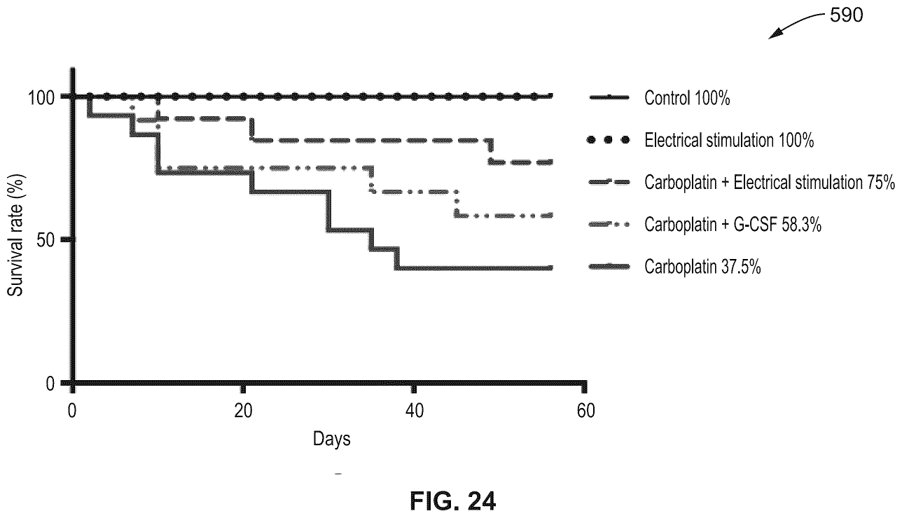

FIG. 24 illustrates an example embodiment 590 of results indicating survival rates of the rats in the same five groups (control, ES, carboplatin, carboplatin+ES, and carboplatin+G-CSF) as previously described after two cycles of carboplatin over a period of 60 days. The figure also depicts the survival rates with the chemotherapy alone (e.g., carboplatin) at a 37.5% survival rate, and chemically augmented chemotherapy (e.g., carboplatin G-CSF) at a 58.3 survival rate; whereas chemotherapy with the electrical stimulation resulted in a 75% survival rate.

The rats in the carboplatin+ES groups had higher survival rates compared to the carboplatin and carboplatin+G-CSF groups. ES is seen according to these tests to reduce the severity of chemotherapy-induced hematology toxicity and treatment-related mortality. Besides the recovery of the neutropenia and thrombocytopenia, electrical stimulation of sympathetic nerves can also decrease the mortality rate after two cycles of carboplatin.

### 3. Mechanism

### 3.1. Electrical Stimulation Preserves Nerve and Bone Marrow (BM) Microenvironment

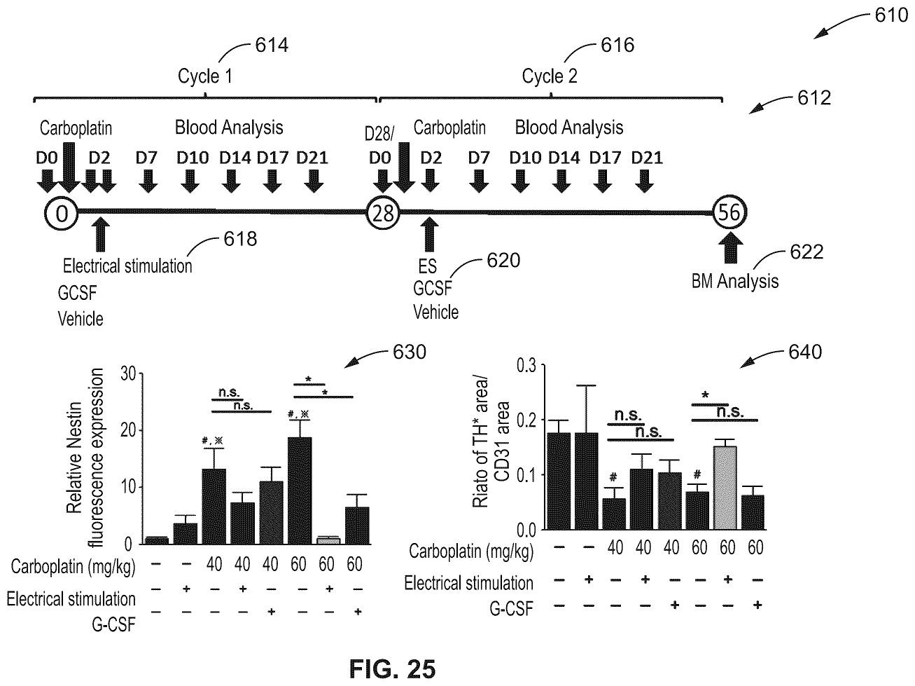

FIG. 25 illustrates example results 610 from the ES augmented treatments 612 in relation to preserving the nerve and bone marrow microenvironments. In the upper portion of the figure is shown the two cycles 614 and 616 of treatments 618 and 620, on day 0 through day 2 and the blood analysis on days 2, 7, 10, 14, 17 and 21, and BM analysis 622.

The testing utilized immunofluorescence staining of nestin for mesenchymal stem cell, tyrosine hydroxylase (TH+) for sympathetic nerve, and CD31 for vascular to evaluate the alteration of bone marrow microenvironment after three cycles of carboplatin.

As seen in the bar graphs 630 and 640 at the bottom of the figure, in comparison to the control group, there was more extensive expression of nestin from the rats receiving the chemotherapy (e.g., carboplatin). Use of carboplatin also resulted in reduced expression of TH+ in the sympathetic nerve; however, this reduction was not observed in the carboplatin+ES group. As for the area of TH+/CD31 evaluated by immunofluorescence staining, the rats in the carboplatin groups expressed lower levels than in the control group, but again this was not observed in the carboplatin+ES group.

The results indicate that exposure to chemotherapeutic agents (e.g., carboplatin) leads to damage of sympathetic nerve and proliferation of mesenchymal stem cells in compensation, and that this can be reversed through ES. These results demonstrated that carboplatin induced the damage of the sympathetic nerve and expansion of nestin+mesenchymal stem cell within bone marrow, which can be reversed by ES.

After two cycles of chemotherapy, exemplified as using carboplatin with different dosages of 40 mg/kg and 60 mg/kg, the bone marrow of the rats from the five experimental groups was analyzed by immunofluorescence to evaluate the alteration of the bone marrow microenvironment. The bone marrow tissue of rats was stained with nestin, TH and anti-CD31 antibody for mesenchymal stem cell, sympathetic nerve and endothelial cell, respectively. Compared to the control group, the area of the sympathetic nerve along with arteriole significantly decreased and the mesenchymal stem cells increased in the rats from the carboplatin group. Electrical stimulation was found to preserve the nerve structure and bone marrow microenvironment injured by chemotherapy.

3.2. Electrical Stimulation of Sympathetic Nerve Reduces Adhesion Molecules within the Bone Marrow Microenvironment

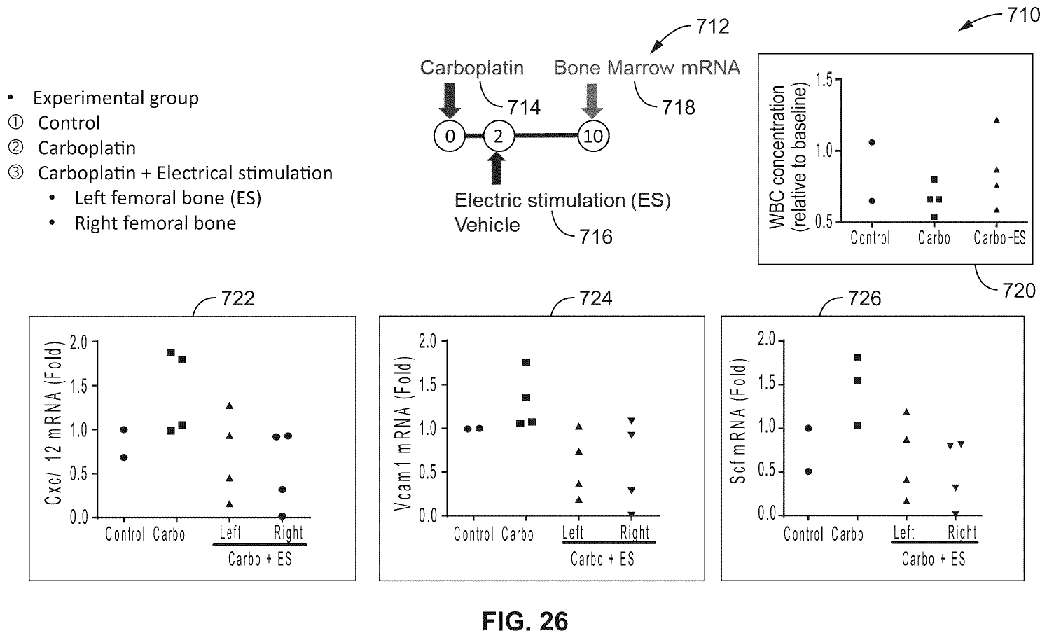

FIG. 26 illustrates an example of results 710 in which ES was found to degrade adhesion molecules within bone marrow and mobilize stem cells. In the upper portion of the figure a testing flow 712 is shown with carboplatin administration 714 at day 0, electrical stimulation (ES) 716 on day 2, and bone marrow mRNA tested 718 at day 10. Plots are shown on the right side and in the lower portion of the figure for white blood cell concentration 720, CXCL12 mRNA 722, Vcam1 mRNA 724 and SCF mRNA 726.

The mRNA level of CXCL12, VCAM1 and SCF within bone marrow were analyzed on the 10th day after carboplatin. There was found to be decreased mRNA level of CXCL12, VCAM1 and SCF in the carboplatin+ES group compared to either the control or carboplatin groups. ES on the left sciatic nerve induced similar results on both sides of the sciatic nerve. The results show that ES can reduce the level CXCL12, VCAM1 and SCF within bone marrow, thus facilitating hematopoietic cell mobilization from bone marrow to the peripheral blood.

3.3. Electrical Stimulation of Sympathetic Nerve Reduced Adhesion Molecules within the Bone Marrow Microenvironment

To study the etiology of recovery of leukopenia and thrombocytopenia after electrical stimulation, the mRNA of several types of adhesion molecules were analyzed which are responsible for the retention of hematopoietic stem cells within bone marrow. The mRNA level of CXCL12, VCAM1 and SCF were evaluated from the bone marrow of the rats receiving carboplatin and carboplatin+electrical stimulation. Both left (the side of electrical stimulation) and right (without electrical stimulation) femoral bones of the same rat from the carboplatin+electrical stimulation group were evaluated to identify whether electrical stimulation induce local or systemic effect. Accordingly, the results seen in FIG. 26 also demonstrates that electrical simulation decreases the mRNA level of CXCL12, VCAM1 and SCF, and mobilizes hematopoietic cells from bone marrow to peripheral blood consequently. Electrical stimulation induces systemic rather than local effects, since there was similar presentation from both femoral bone marrow of the same rats.

### 3.4. Electrical Stimulation of Sympathetic Nerve Promotes Hematopoietic Regeneration

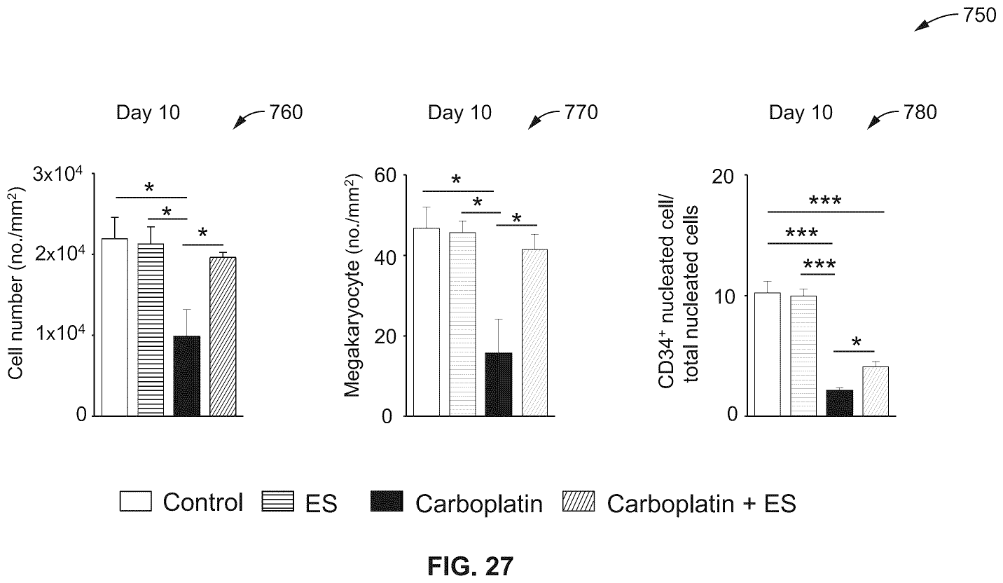

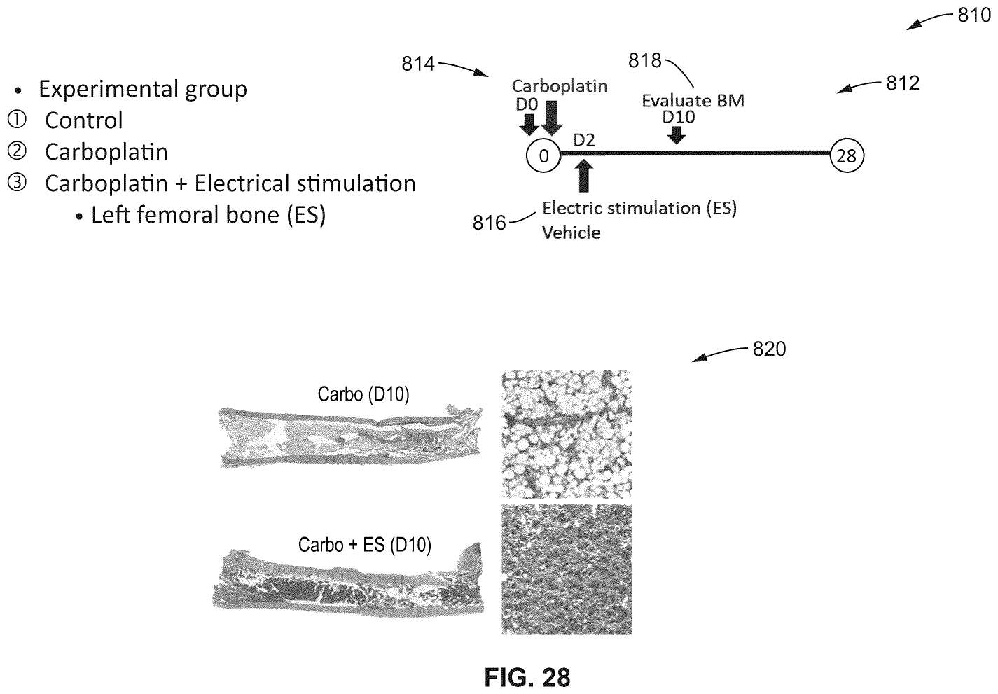

FIG. 27 and FIG. 28 illustrate example results 750, 810 and 820 of ES promoting hematopoietic regeneration.

In FIG. 27 is shown bar graphs of Cell number 760, megakaryocytes 770 and CD34 ratio of nucleated cells to total nucleated cells 780 for a control group, ES group, carboplatin group and carboplatin+ES group, shown at day 10 of testing. In FIG. 28 the testing profile is shown 812 with carboplatin 814 followed by electrical stimulation 816, evaluating BM 818 at day 10 in a 28-day cycle. The lower portion of the figure depicts images 820 showing an image and associated close up magnification of the bone marrow for the carboplatin group (upper images) and the carboplatin+ES group (lower images).

As seen in these figures, after carboplatin treatment, the cellularity of bone marrow was significantly increased in the carboplatin+ES group, compared to the carboplatin only group. These findings indicated that ES promotes hematopoietic regeneration.

On the 10th day after carboplatin administration, the counts of total cell number, megakaryocytes and CD34+ precursor cells were significantly higher in the carboplatin+ES group compared to the carboplatin group. This data demonstrates that ES promotes hematopoietic regeneration after chemotherapy.

3.5. Electrical Stimulation of Autonomic Nerve Alters Gene Expression Profile within the Bone Marrow Microenvironment

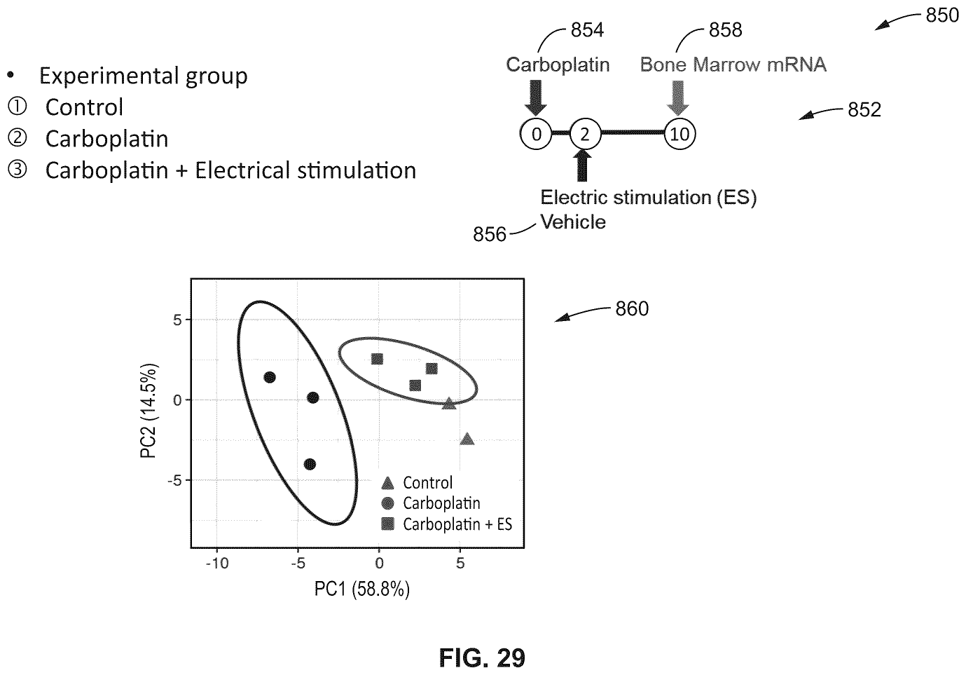

FIG. 29 illustrates example results 850 in which the addition of ES was shown to alter the gene expression profile within the bone marrow. The treatment schema 852 is shown with carboplatin administration 854 at day 0, followed by ES vehicle 856 at day 2, with bone marrow RNA sequencing performed 858 on the control, carboplatin and carboplatin+ES groups at Day 10 after chemotherapy.

The lower portion of the figure depicts a clustering analysis plot 860 of the control group, carboplatin only group, and carboplatin+ES group using Principle Component Analysis.

To identify the alteration of the genetic signature after electrical stimulation, bone marrow bulk mRNA-sequencing of the rats was performed for the control, carboplatin and carboplatin+ES groups. The plot 860 demonstrates that the gene modulating cell migration and activation revealed different gene expression levels among the rats of different groups. It can be seen that the data points for carboplatin+ES are clustered close to the control group while is separated from the carboplatin group using Principal Component Analysis—PC1 and PC2. The above demonstrates ES is able to alter the genetic signature which modulates cell migration and activation, and thus facilitate recovery.

### 3.6. Indirect Nerve Stimulation

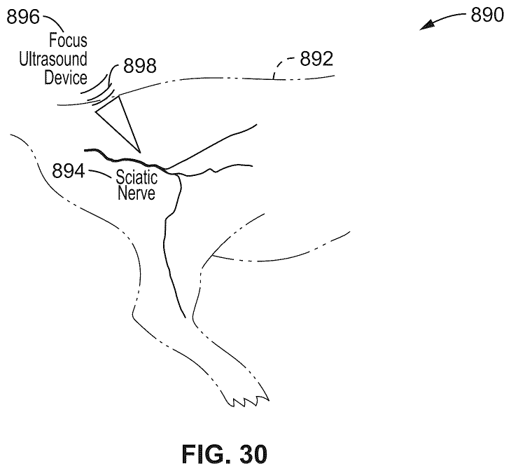

FIG. 30 illustrates an example 890 of indirect nerve stimulation, as described in Section 1.3, that may be utilized in the present disclosure. In some applications rather than directly stimulating the nerve with electrical signals passing through the electrode(s), the stimulation can be indirectly created. In the example shown a focused ultrasonic beam(s) 898 from an ultrasonic device 896 is direct at the nerve 894 (e.g., such as the sciatic nerve shown) being innervated on test subject 892.

### 4. Conclusion on First Section

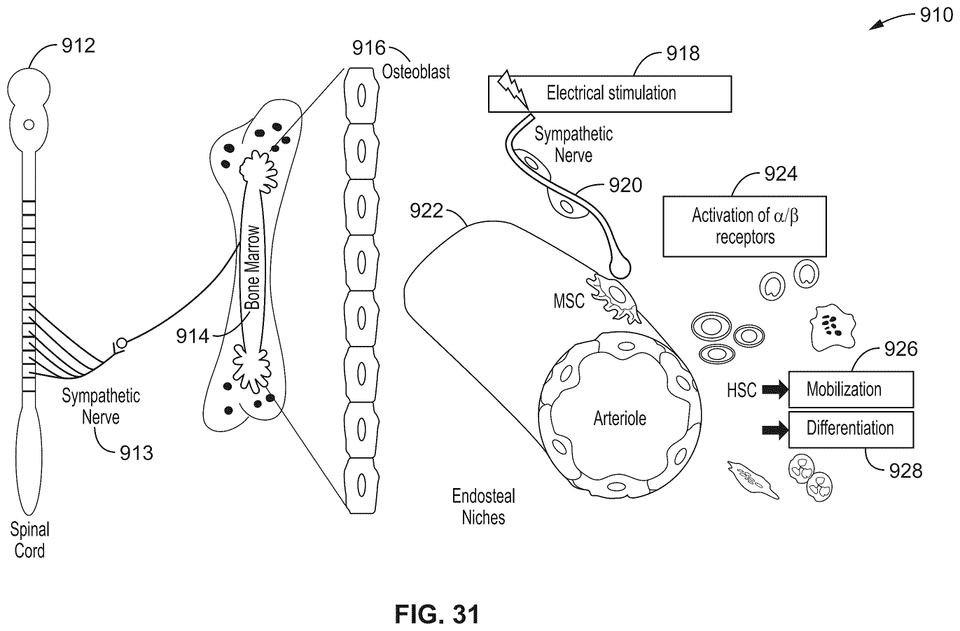

FIG. 31 illustrates an example embodiment 910 showing how the use of electrical stimulation (ES) modulates hematopoiesis and bone marrow microenvironment. Sympathetic nerves 913 from the spinal cord 912, begin at the first thoracic vertebra of the vertebral column and extend to the second or third lumbar vertebra. The postsynaptic sympathetic nerves enter into bone marrow 914 to regulate bone marrow niche. Electrical simulation 918 of sympathetic nerve 920 within bone marrow can activate the adrenergic receptors 924. on arteriole 922, to promote differentiation 928 and facilitate mobilization 926 of Hematopoiesis Stem Cell (HSC), which alleviates chemotherapy-related hematologic toxicity.

In conclusion, through electrical stimulating of bone marrow sympathetic nerve, the apparatus and method according to the present disclosure is able to promote hematopoietic mobilization and regeneration, which reduces chemotherapy-induced hematologic toxicity. ES can also rescue sympathetic nerves from chemotherapy-related injury and preserve the bone marrow microenvironment.

### 5. Biology Basis

### 5.1. Introduction to Pain Physiology

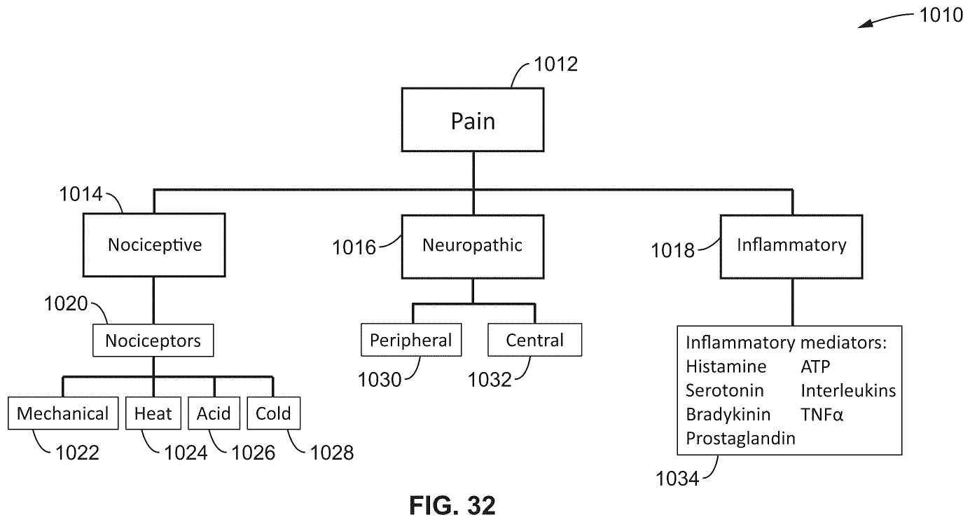

in FIG. 32 is illustrated 1010 a classification of pain. Pain 1012, commonly defined as “an unpleasant sensory and emotional experience associated with actual or potential tissue damage”, has become one of the major challenges of the modern healthcare system. Pain can be classified, according to its mechanism, as nociceptive pain 1014, inflammatory pain 1018, and neuropathic pain 1016, as shown in the figure. Nociceptive pain refers to the pain generated due to activation of nociceptors 1020 in cutaneous, somatic, or visceral structures. The nociceptors are activated by tissue injury and the subsequent pain acts as a physiological alarm system and is therefore usually ‘adaptive’. The nociceptors can be responsive for example to mechanical 1022, heat 1024, acid 1026 and cold 1028 conditions. Inflammatory pain refers to the pain generated due to inflammatory mediators 1034 being released following tissue injury and systemic or local inflammation. In contrast, neuropathic pain 1016 is the pain arising as a direct consequence of a lesion or disease affecting the somatosensory nervous system either in the peripheral nervous system (PNS) 1030 or in the central nervous system (CNS) 1032, which is described as a maladaptive response.

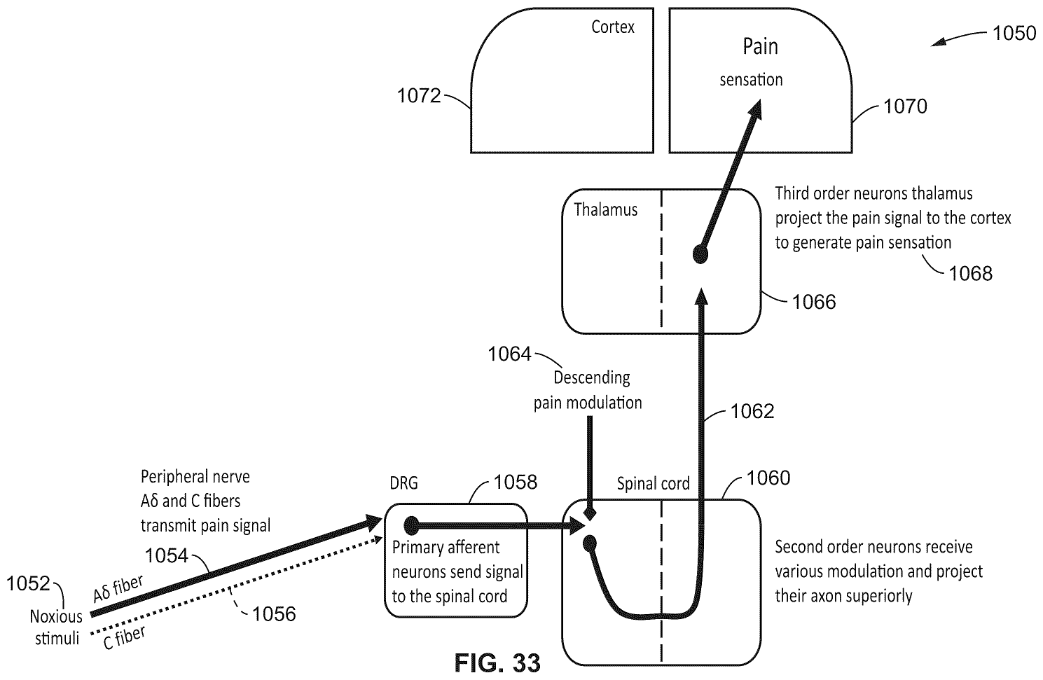

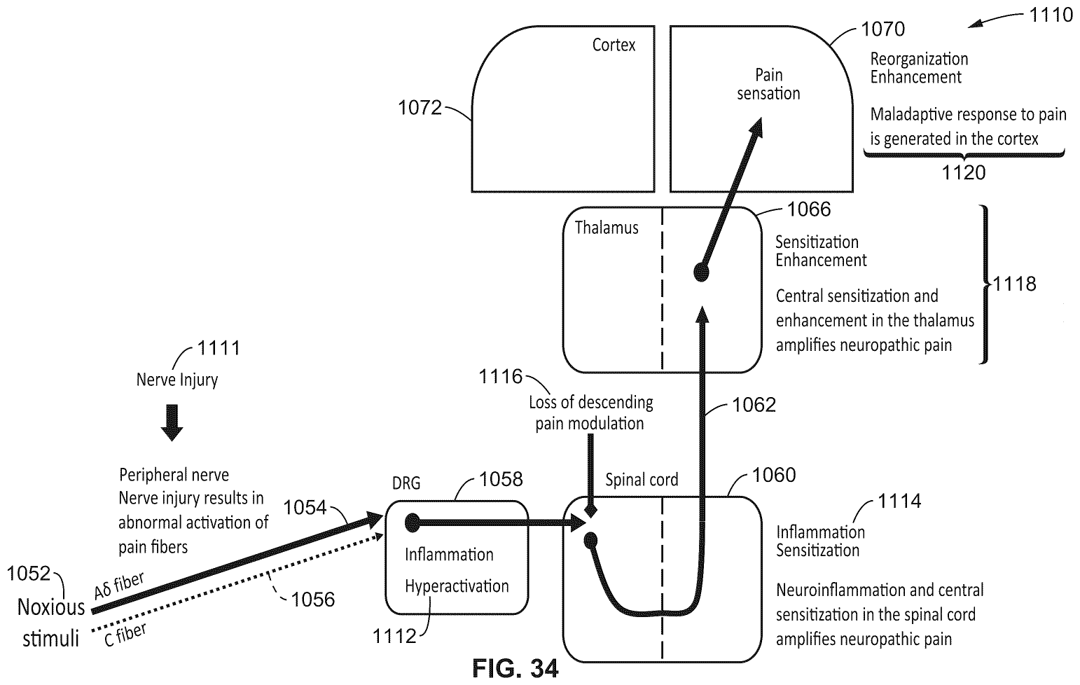

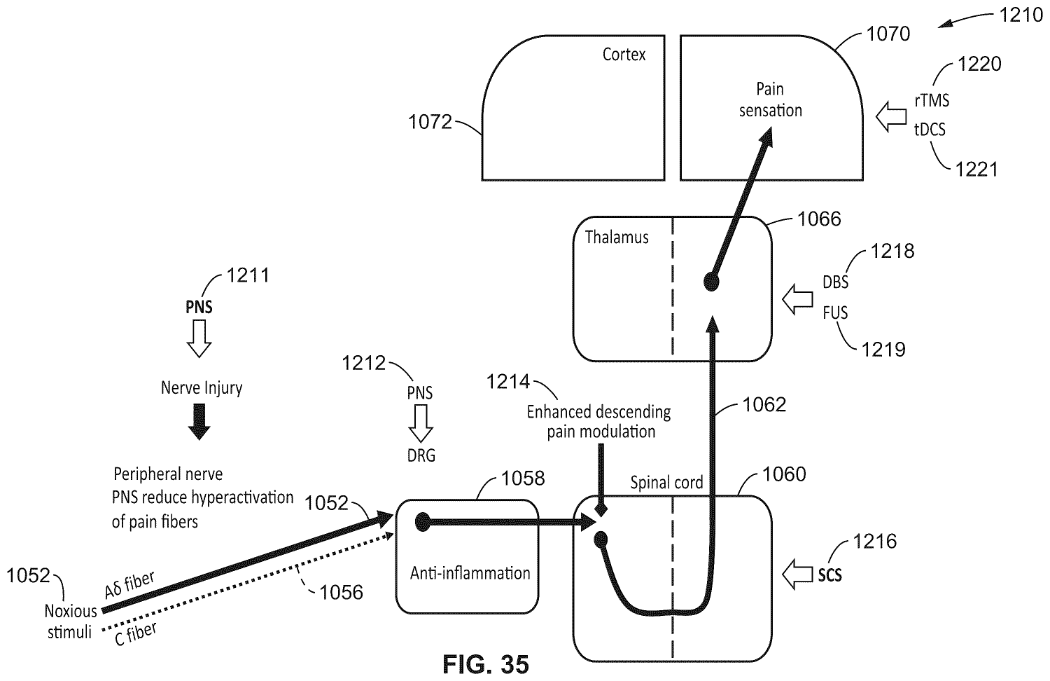

FIG. 33 through FIG. 35 shows operation of the neuron pathways while FIG. 34 depicts cases of nerve injury, and FIG. 35 shows application of peripheral nerve stimulation (PNS) when there is a nerve injury.

FIG. 33 illustrates neuronal circuitry and the pathway of pain 1050 in response to a nerve injury. The primary afferent neurons and the spinothalamic tract are the key transmitters of pain signals. As shown in the figure, the electrochemical signal arising from the nociceptors are noxious stimuli 1052 transmitted through the AS fibers 1054 and the C fibers 1056 of the primary afferent neurons. The cell bodies of the primary afferent neurons reside in the dorsal root ganglion (DRG) 1058, and synapse onto second order neurons located within the dorsal horn of the spinal cord 1060.

In addition to the signals received from primary afferent neurons, second-order neurons in the dorsal horn of the spinal cord 1060 also receive input from the descending pain modulation pathway 1064. Neurons originating from the rostral ventral medulla-periaqueductal gray matter send descending projections to these second-order neurons.

The second-order neurons integrate signals from both the primary afferent neurons 1058 and the descending pain modulation pathway 1064. They combine these inputs and transmit an output signal by crossing over (decussating) and projecting their axons superiorly in the anterolateral region of the spinal cord, forming the spinothalamic tract. Neurons within the spinothalamic tract 1066 synapse with third-order neurons in the ventral posterior nucleus of the thalamus (1066), which further relay the pain signal 1068 to the primary somatosensory cortex.

It should be appreciated that cortex 1072 is an anatomical location, whereas the pain sensation 1070 is a neuronal event occurring in the cortex. The dashed lines in 1060 and 1066 indicate a midline, since pain signals travel the contralateral side of the cortex.

### 5.2. Neurophysiology of Neuropathic Pain

In FIG. 34 is illustrated 1110 the pathway of pain in response to a peripheral nerve injury 1111 which results in abnormal activation of the pain fibers. Compared with the previous figure, at the dorsal root ganglion (DRG) 1058 there is now inflammation and hyperactivation. There is a loss 1116 of descending pain modulus at the spinal cord with inflammation sensitization 1114 in which there is neuroinflammation and central sensitization in the spinal cord which amplifies neuropathic pain. At the Thalamus there is sensitization enhancement 1118. The pain sensation 1070 has reorganization enhancement 1120 of maladaptive response to pain generated in the cortex.

Neuropathic pain (NP), defined as pain caused by a primary lesion of the nervous system, is characterized by abnormal activation of pain conducting pathways. Mechanical allodynia and thermal hypersensitivity are commonly observed in patients with NP and animal models of NP, including rats with L5 nerve root ligation (NRL). Dysregulated pain signaling and modulation in the central nervous system (CNS) play a critical role in NP. After NRL, the pathogenesis of NP is characterized by neuroinflammation in the spinal cord dorsal horn (SCDH) at the corresponding level. Injury of a peripheral nerve has been shown to induce proliferation and hypertrophy of spinal cord astrocytes via activation of the mitogen-activated protein kinase signaling pathway. Activation of extracellular signal-regulated kinase in spinal microglia and astrocytes following spinal nerve injury has also been reported. Such changes in spinal cord glial cells are known to facilitate hyperactivation of somatosensory neurons in the SCDH and result in NP. Together, these findings suggest a pivotal role of CNS neuroinflammation in the development of acute pain hypersensitivity and the subsequent transition from acute to chronic NP.

### 5.3. Innervation and Regulation Mechanisms—Neuromodulation

FIG. 35 illustrates the application of neuromodulation in pain conditions as were shown in FIG. 34. Neuromodulation (electrical) techniques, such as peripheral nerve stimulation (PNS) 1212 and spinal cord stimulation (SCS), have been used to treat debilitating pain in patients who fail to respond to, or are unable to tolerate pharmacological treatments. Emerging clinical evidence supports the clinical efficacy of neuromodulation in the management of chronic pain, including the use of transcutaneous electrical nerve stimulation, dorsal root ganglion stimulation, spinal cord stimulation (SCS), deep brain stimulation (DBS) 1218 or focused ultrasound (FUS) 1219 at the thalamus 1066, or repetitive transcranial magnetic stimulation (rTMS) 1220, and transcranial direct current stimulation (tDCS) 1221 at the cortex 1072. Some researchers have reported a reduction in pain scores of more than 50% and a decrease in the doses of required analgesics in a large number of NP patients receiving PNS. Similar results were obtained in patients with chronic back pain, who had lower pain scores and analgesic consumption after PNS. Previous studies have demonstrated that PNS can increase the pain threshold and shed light on potential mechanisms of PNS, such as gate-control induced paresthesia, inflammatory modulation, and endogenous pain inhibition pathways. It was reported that PNS at a frequency of 100-10000 Hz facilitated gate-control induced paresthesia to achieve analgesia. On the other hand, lower frequency (2-30 Hz) stimulation is reported to have a modulatory effect on neuroinflammation in the peripheral and central nervous system. It is important to note that the effectiveness heavily depends on the stimulation protocols as addressed in Section 5.1.

### 6. Animal Experiments

### 6.1. Validation of the Therapeutic Effects of PNS

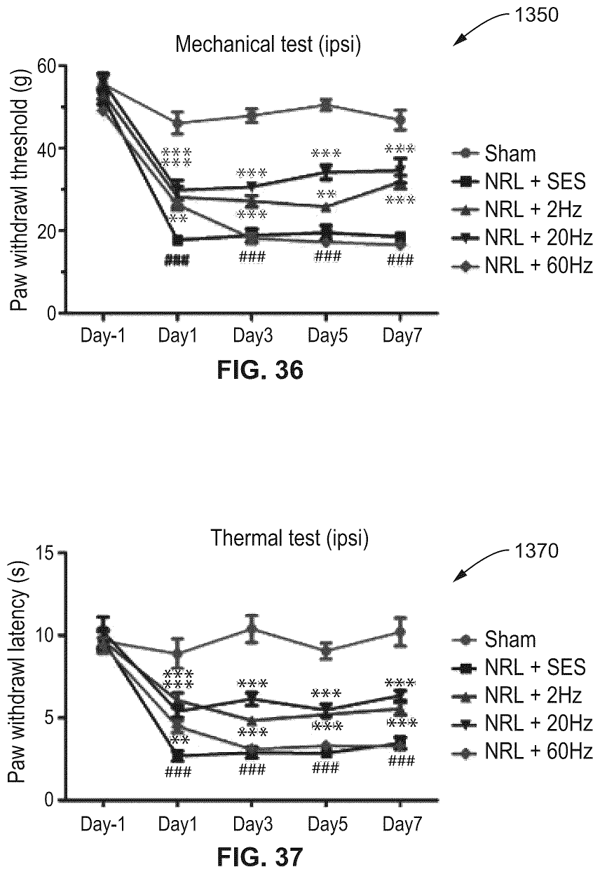

FIG. 36 through FIG. 37 illustrate results from animal experiments to validate the therapeutic effects of PNS.

Animal experiments were conducted using rats with right L5 nerve root ligation (NRL) and Sciatic Nerve Stimulation (SNS). Ipsilateral SNS was performed at 2 Hz, 20 Hz, and 60 Hz frequencies. Behavioral tests were performed to assess pain and thermal hypersensitivity before and after NRL and SNS. Expression of inflammatory proteins in the L5 spinal cord and the immunohistochemical alterations of spinal cord astrocytes and microglia were examined on post-injury day 7 (PID7) following NRL and SNS.

In FIG. 36 are shown a mechanical test (ipsi) 1350 of paw withdrawal threshold with respect today for a control (sham) and for NRL with Sham Electrical Stimulation (SES), then showing NRL with Sciatic Nerve Stimulation (SNS) at frequencies of 2 Hz, 20 Hz and 60 Hz.

In FIG. 37 a plot 1370 is also shown of a thermal test depicting paw withdrawal latency (seconds) in each of these cases over the experimental period.

It is seen in the figure that with SES the rats with L5 injury showed a decreased pain threshold and latency on the von Frey and Hargreaves tests (paw withdrawal threshold and paw withdrawal latency tests). Both 2 Hz and 20 Hz SNS alleviated pain behavior and hyperactivation of Spinal Cord Dorsal Horn (SCDH) neurons. On Post-Injury Day 7 (PID7), NRL resulted in increased activation of spinal cord inflammatory cells.

In addition, 2 Hz and 20 Hz SNS suppressed the activation of spinal cord astrocytes and microglia following NRL on PID7. Activity of the descending serotonergic pain modulation pathway showed an increase early on PID1 following 2 Hz and 20 Hz SNS. These animal experimental results support that both 2 Hz and 20 Hz SNS can alleviate NP behaviors and hyperactivation of pain conducting pathways. SNS may regulate neuroinflammation and reduce inflammatory protein expression, astrocytic gliosis, and microglia activation. Our preclinical animal investigations showed the efficacy of neuromodulation to alleviate neuropathic pain and neuroinflammation in a rat model on nerve injury.

### 6.2. Validation of Stereotactic Electric Nerve Stimulation

Both invasive and transcutaneous approaches were conducted for animal experiments using rats with right L5 NRL. FIG. 36 as discussed above shows animal experiments using hook electrodes at the sciatic nerve. Ipsilateral SNS was performed at 20 Hz. Behavioral tests were conducted to assess pain and thermal hypersensitivity before and after NRL and stereotactic SNS respectively. The immunohistochemical alterations of L5 spinal cord astrocytes and microglia were examined on Post-Injury Day 7 (PID7) following NRL and stereotactic SNS respectively.

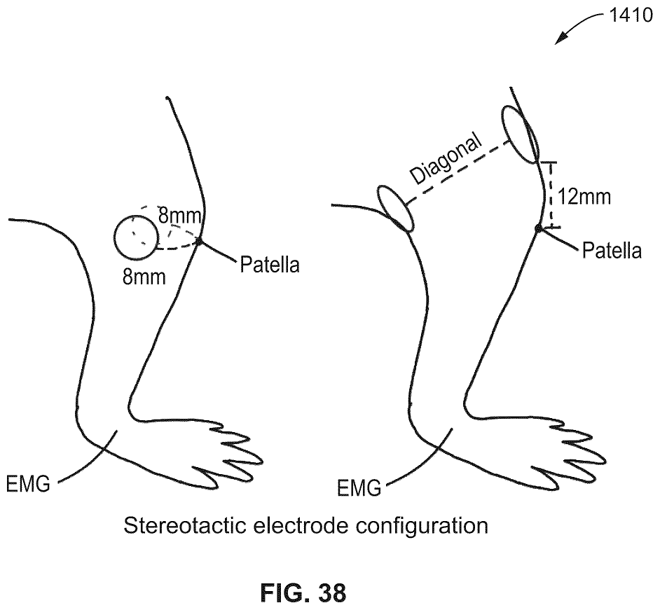

In FIG. 38 is illustrated an example of stereotactic electrode configuration 1410, with the location of electrode pairs for stereotactic electric stimulation.

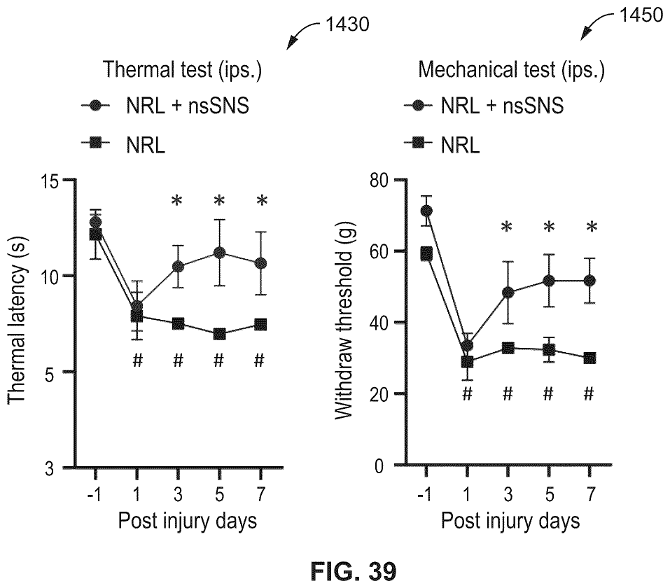

In FIG. 39 are plots indicating thermal 1430 and mechanical testing 1450 of thermal latency and withdrawal threshold comparing NRL+nsSNS, and NRL.

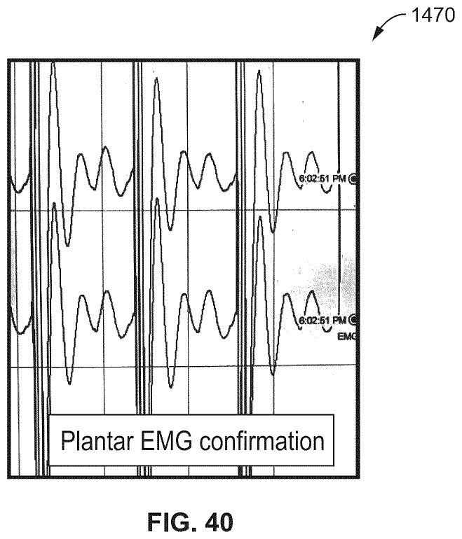

FIG. 40 are plots 1470 of Plantar Electromyography (EMG) signals recorded to confirm and fine-tune the stimulation parameters. In the animal pain behavior experiments, rats with L5 injury showed a decrement in both pain threshold and latency in the von Frey and Hargreaves tests. In contrast to this, the application of stereotactic stimulation alleviated the painful behaviors showing increments in both pain threshold and latency in both the von Frey and Hargreaves tests. Further, examination showed the elevated activation of spinal cord inflammatory cells following nerve injury was also reduced after the application of stereotactic electric nerve stimulation. This experiment validated the benefit of stereotactic electric nerve stimulation with proper stimulation protocols.

### 7. Methods, Techniques, and Stimulus Protocols

The present disclosure presents the electrical neuromodulation (bioelectronics medicine) methods and techniques for neuropathic pain relief. The present technique is called the Peripheral Neuromodulation Pain Alleviation (PNPA) system. The devices and methods are more completely described in the following.

### 7.1. Stimulus Specifications

Targeted nerves include:
Upper extremity: radial nerve
Lower extremity: sciatic nerve and tibial nerve

Frequency: range 1-100 Hz

### Waveform:

(1) Monophasic and biphasic traditional uniform pulse train.
(2) Monophasic and biphasic pulse train with varying pulse arrival time.
(3) Monophasic and biphasic burst pulse train—duty cycle consisting of on time (high frequency pulse train) and off time.
(4) Biomimetic waveforms that emulate biological signals such as EMG, EKG, and pulse trains with pulses appear at stochastic time (i.e., non-periodical arrival times).

Voltage: 0.1V to 30V

Amplitude: 0.1 to 50 mA

Pulse width: 50 to 2000 μsec

### 7.2. Treatment Protocol

### 7.2.1. Exemplary Embodiment of PNPA on Sciatic Nerve

Frequency: range 1-100 Hz

### Waveform:

(1) Monophasic and biphasic traditional uniform pulse train.
(2) Monophasic and biphasic pulse train with varying pulse arrival time.
(3) Monophasic and biphasic burst pulse train—duty cycle consisting of on time (high frequency pulse train) and off time.
(4) Biomimetic waveforms that emulate biological signals such as EMG, EKG, and pulse trains with pulses appear at stochastic time (i.e., non-periodical arrival times).

Voltage: 0.1V to 30V

Amplitude: 0.1 to 50 mA

Pulse width: 50 to 2000 μsec

### 7.2.2. Exemplary Treatment Protocol Specifications:

Timing of intervention: application of the PNPA system is initiated preferentially within 24-hours or as soon as possible after onset of lower extremity pain in the case of sciatic nerve stimulation.

Treatment course: each treatment course consists of 1-2 cycles of sciatic nerve stimulation.

Treatment cycle: one cycle includes 1-3 sessions of sciatic nerve stimulation, while each session is separated by 2 days, and each cycle is separated by an interval of one week.

Duration: each session comprises 1-hour sciatic nerve electric stimulation.

Evaluation: pain scores(behaviors) are evaluated at the end of each cycle in the treatment protocol.

### 7.3. Peripheral Neuromodulation Pain Alleviation (PNPA) System

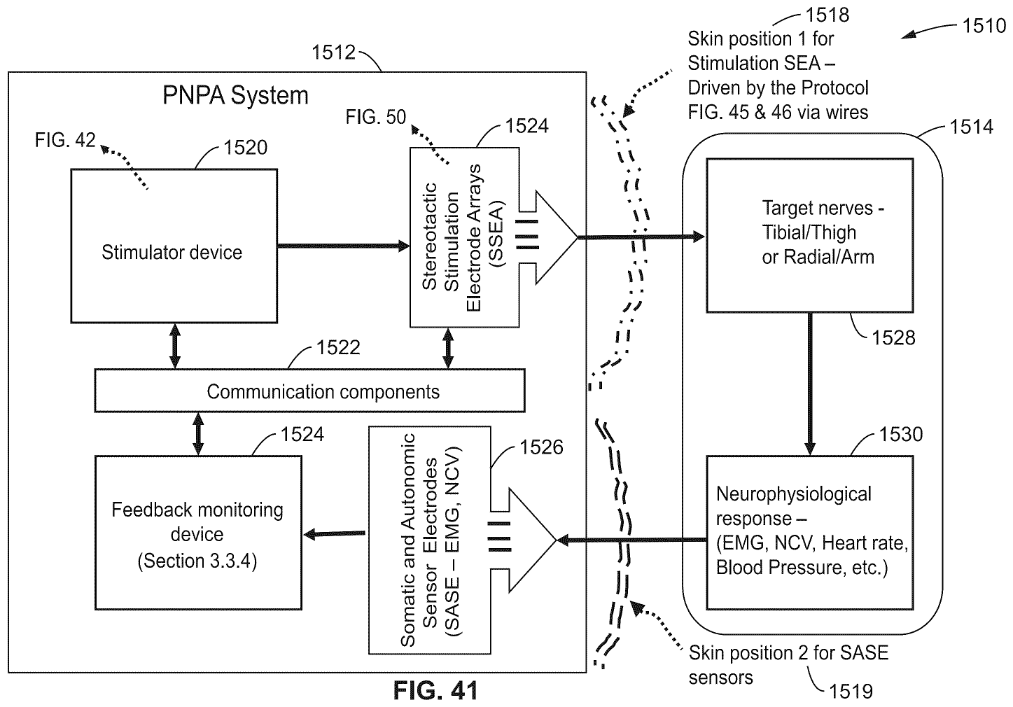

FIG. 41 illustrates an example embodiment 1510 of the PNPA system. In one embodiment of the disclosed PNPA system, the devices and methods are more completely described in the following. The devices and methods are directed towards producing therapeutic effects using an energy source to transmit electric energy to peripheral nerves via a non-invasive manner. The PNPA system can transmit electric energy directed to, or near peripheral nerves in the body and extremities in a patient to modulate or stimulate the targeted nerves to produce therapeutics effects including but not limited to pain modulation, analgesia, paresthesia, and modulation of neuroinflammation.

The major differences between the PNPA system of this disclosure and the currently commercially available spinal cord stimulation techniques include the following. (1) PNPA uses stimulation signals delivered on the peripheral nerves such as the sciatic and tibial nerve in contrast to the spinal cord. (2) PNPA delivers stimulation signals in a non-invasive manner, through the stereotactic electrode array applied on the body surface in proximity to the target nerve. (3) PNPA stimulation signals can consist of different waveforms including biomimetic waveforms, in contrast to the high-frequency pulse trains used in spinal cord stimulators.

In the PNPA system of this disclosure, the stereotactic electrode array is specially designed to target the nerves at the thigh and arm. The optimization method utilized is able to obtain montages of stimulation which are capable of selectively focusing on the radial nerve for upper extremity and the sciatic and tibial nerve for the lower extremity.

In the PNPA system shown in FIG. 41, the neuromodulation system 1512 is composed of a stimulator device 1520, a set of Stereotactic Stimulation Electrode Arrays (SSEA) 1524, at a first skin position 1518. A feedback monitoring device 1524 receiving input from Somatic and Autonomic Sensor Electrodes (SASE) at a second skin position 1519, such as for receiving EMG and Nerve Conduction Velocity (NCV)) 1526. Also, there are communication components (wired and/or wireless) 1522 to interconnect the devices.

The PNPA system is directed into a target 1514, in particular tibial (thigh) or radial (arm) 1528, and for collecting neurophysiological response 1530 (EMG, NCV, heart rate, blood pressure, and so forth).

The stimulator device in the PNPA system is comprised of a graphic user interface, microcontroller firmware, and a current/voltage driver (generator) differentiates the hardware. The SSEA are comprised of multiple ring structured electrode arrays, which include stimulation electrodes and somatic/autonomic sensor electrodes. The stimulation electrodes deliver the generated electric signal from the stimulator device to the target nerve in the arm/thigh. The neurophysiological responses following the stimulation are recorded by the somatic/autonomic sensor electrodes (SASE). The somatic/autonomic sensor electrodes provide feedback signal to the feedback monitoring device.

### 7.3.1. Stimulator Device

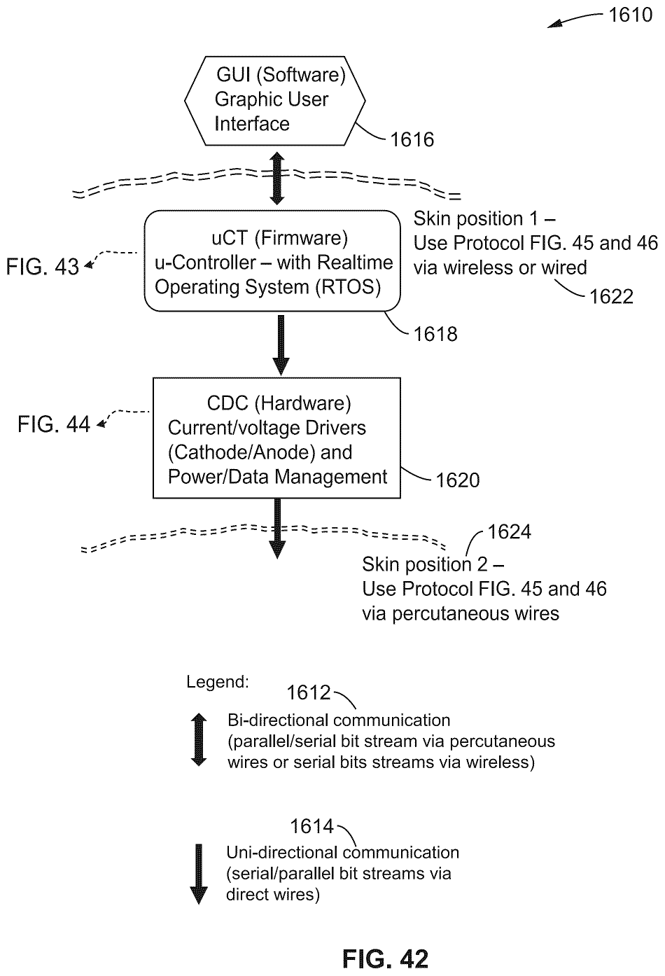

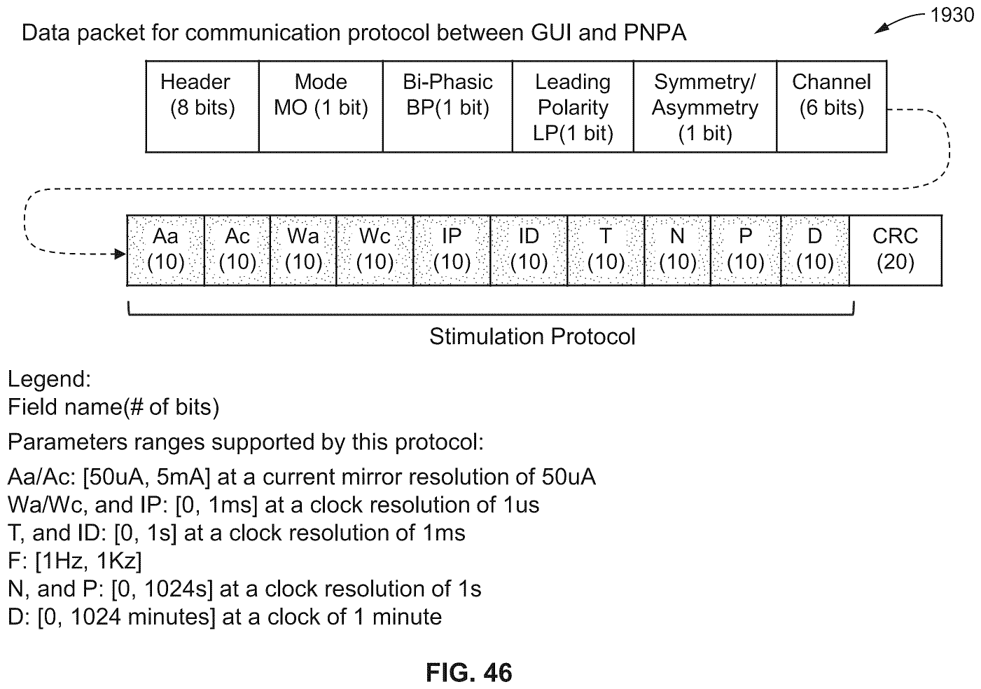

FIG. 42 illustrates an example embodiment 1610 of the PNPA system having a Graphic User Interface (GUI) 1616, a microcontroller with firmware (uCT) 1618, and current/voltage driver hardware (CDC) 1620. It will be noted that the GUI is connected to the uCT with a bi-directional communication link 1612, whereas the uCT is coupled to the CDC level of hardware with a unidirectional link 1614. The main functions of the circuitry and the dedicated microcontroller firmware are to faithfully decode a protocol (FIG. 46) specified by the GUI and convert it into an electrical stimulation waveform (e.g., pulse train of biphasic current pulses). In addition, the GUI implements the algorithm described in Section 7.3.2 to obtain the optimal stimulation current montage and its information is also included in the protocol.

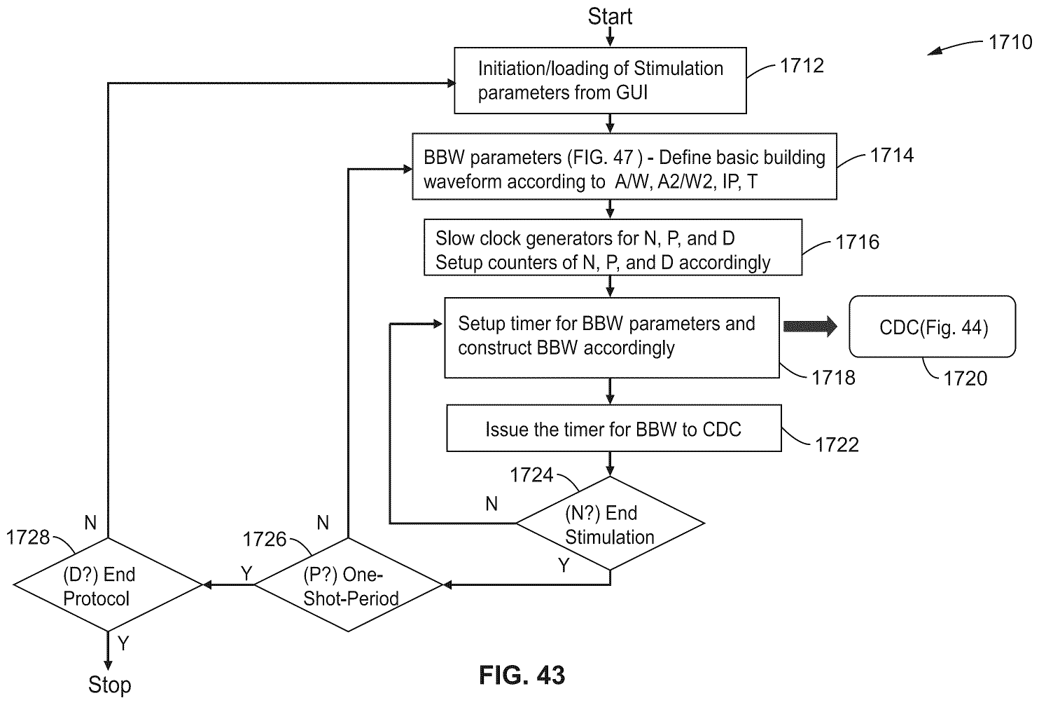

FIG. 43 illustrates an example embodiment 1710 of process flow on the microcontroller-based implementation of uCT using a Real-Time Operating System (RTOS). Protocol commands are issued from the GUI to the uCT to initialize and load 1712 stimulation parameters.

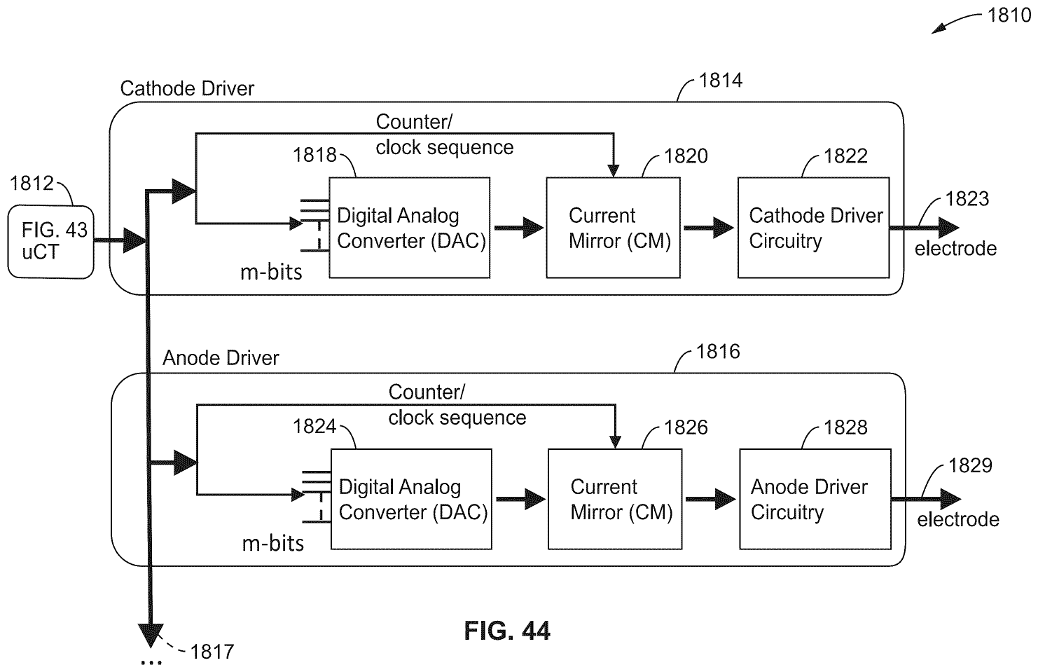

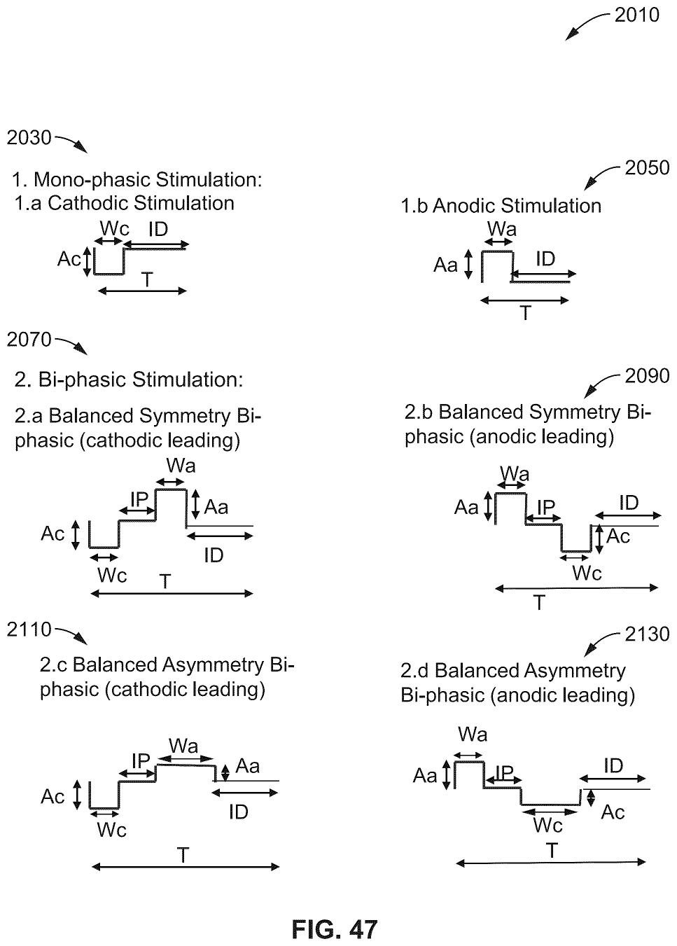

The uCT decodes these commands and generates 1714 Basic Building Waveform (BBW) parameters according to A/W, A2/W2, IP, and T as depicted in FIG. 47. It configures 1716 the slow clock generators and counters for N, P, and D, and sets up timers 1718 for BBW parameters for the CDC (which is shown in FIG. 44). The uCT then issues 1722 the timer for BBW to the CDC.

Check 1724 determines if intraburst (N) stimulation parameters have been completed. It will be noted that each stimulation pulse train can have on (N) times in which stimulation waveforms are generated, and off times in which no stimulation is generated. If there are more on (N) time stimulation parameters to be addressed, then execution moves back to block 1718 for another pass.

Otherwise, check 1726 is performed to determine if the stimulation pulse train (burst) period (P) parameters are completed. If they are not, then execution returns to block 1714.

Otherwise, check 1728 determines if the total stimulation duration has been built up. If it has been completed, then this processing ends, otherwise execution returns to block 1712. It will be noted the hierarchy of checks 1724, 1726 and 1728, which allows configuring multiple intrabursts (N) within a given one-shot period (P) and multiple one-shot periods before the end (D) of the protocol.

Accordingly, the uCT decodes the protocol and establishes clock generators and timers based on basic building waveform (FIG. 47) to produce the control signals for CDC (FIG. 44) such that the electric simulation waveform is generated.

FIG. 44 illustrates an example embodiment 1810 of CDC hardware which generates both cathodic and anodic electric pulses for the stereotactic stimulation electrode arrays, which subsequently produce an electric field and/or current to induce anterograde and/or retrograde membrane potential propagations in the targeted peripheral nerve. By way of example and not limitation, only one pair of drivers is depicted. It should be appreciated, however, that the present disclosure can support any desired number 1817 of such driver pairs.

The electric pulses to be generated from CDC hardware are controlled and adjusted by the uCT firmware 1812 (seen in FIG. 43) and all the stimulation parameters are displayed in the GUI unit in a real-time manner for clinicians to operate the PNPA system. By way of example, using the communication protocol outlined at FIG. 46, the GUI issues a command to the controller (uCT) to initiate the loading of stimulation parameters and setup of the stimulus by the CDC to the targeted nerve through the electrode (EA) as shown in FIG. 43.

The Current Driver Circuitry (CDC) supports both anode and cathode current stimulation waveforms. Each driver is composed of a Digital to Analog Converter (DAC) 1818, 1824 of m bits (based on the desired output resolution) coupled to a current mirror 1820, 1826. Thus, output from the current mirror induces an output current amplitude of Aa within anode driver circuitry 1828 to be output 1829, and a current amplitude of Ac within cathode driver circuitry 1822 to be output 1823. The pulse width of each anode and cathode pulse is respectively specified by Wa and Wc, whose resolution is limited by the clock frequency.

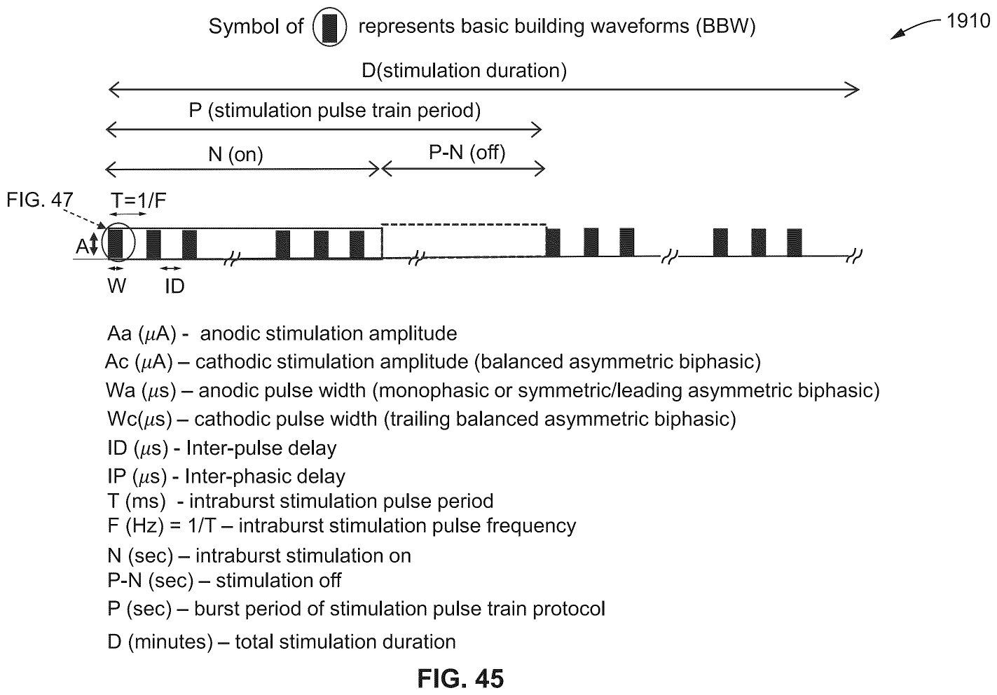

FIG. 45 illustrates an example embodiment 1910 of the waveforms of a treatment protocol. The parameters that specify the stimulation waveforms including specification of polarity and mode including LP (leading cathodic or anodic), MO (voltage or current), BP (biphasic), SY (symmetric or asymmetric biphasic); amplitudes, pulse widths, and delay time—Aa, Wa, Ac, Wc, IP, ID, T, N, P, and D. A pulse train (PT) is composed of a series of N (Counter N) basic building waveforms (BBWs). The One-Shot-Protocol (OSP) is in turn composed of a Pulse Train (PT) and followed by an idle latency of P-N (Counter P). The OSP is repeatedly generated until counter D is expired.

One embodiment with firmware implementation on a micro-controller (uCT) produces the desired stimulus by generating the proper timing sequence for CDC accordingly. The timing and counters of T, N, P, and D for controlling the stimulation parameters are set up and controlled by uCT to produce the proper waveforms defined by the parameter set. The basic timing is set to produce the basic building waveform (BBW) block of the pulse trains defined by N and P counters. Moreover, the uCT is allowed to change the basic building waveform block of the pulse trains every P period. It will be noted that the resolution of the parameters in the time domain is limited by the system clock frequency. The counters of T, N, P, and D are updated according to the uCT clock or corresponding slow clocks derived by the uCT. As an example, the resolution of 0.1 μs is achieved on a uCT with a clock of 10 MHz. The clock generators within the uCT are programmed to produce slower clocks for the counters. The device architecture can provide a wide range of parameters for each individual patient subject.

FIG. 46 illustrates an example embodiment 1930 of an example data packet format for communication between the GUI and the PNPA having the following fields. A header field (e.g., 8 bits) which contains basic information on the command, a mode field (MO) (e.g., 1 bit), a Bi-Phasic field (e.g., 1 bit), a Leading Polarity (LP) field (e.g., 1 bit), a Symmetry/Asymmetry field (e.g., 1 bit), and a Channel field (e.g., 6 bits), after which is a stimulation protocol field containing sub-fields of Aa, Ac, Wa, Wc, IP, ID, T, N, P, D, each of which are exemplified here as being 10 bits. The final field is shown as a Cyclic Redundancy Check (CRC) field, which provides an error checking mechanism so the receiver can determine if the packet has been received without errors, this is depicted using a 20-bit CRC, although other forms of error checking can be utilized without departing from the teachings of the present disclosure.

FIG. 47 illustrates example embodiments of elements within a set of basic building waveforms (BBW) used by the system in generating the stimulus protocols. The BBW can be realized by either micro-controller firmware or hardware, such as System-on-Chip (SoC). Mono-phasic stimulation is shown with both cathodic stimulation 2030 and anodic stimulation waveforms. Bi-phasic stimulation is shown having types with Balanced Symmetry Bi-phasic (cathodic leading) 2070, Balanced Symmetry Bi-phasic (anodic leading) 2090, Balanced Asymmetry Bi-phasic (cathodic leading) 2110, and Balanced Asymmetry Bi-phasic (anodic leading) 2130.

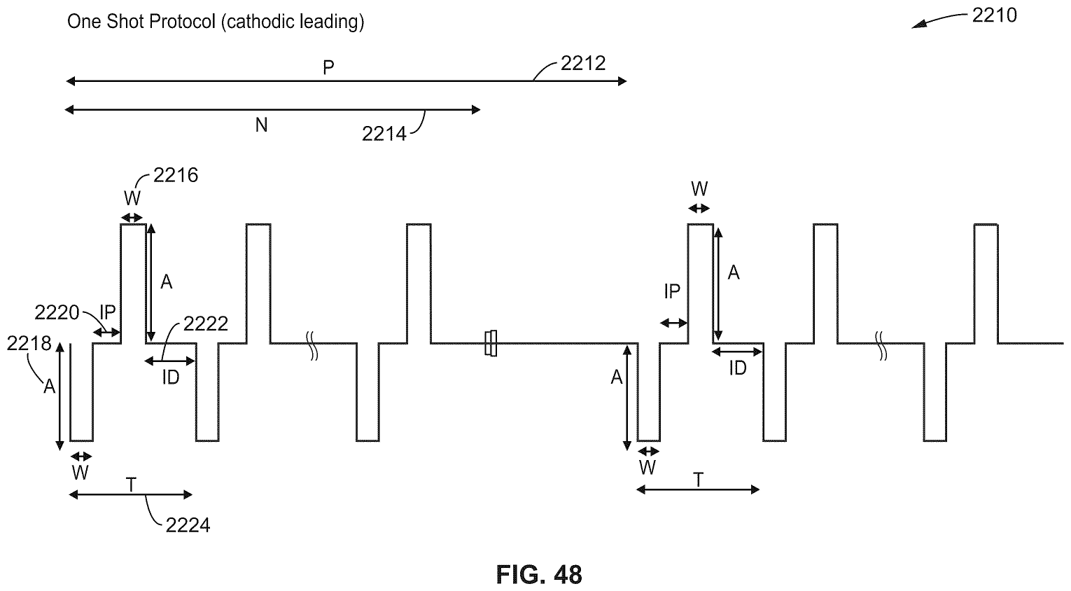

FIG. 48 illustrates an example implementation 2210 of the stimulation protocol of a balanced symmetric biphasic waveform. Note the following on and off times are not shown to scale and are only utilized for demonstrating what has been described in the previous sections. For example, the one-shot cathodic leading stimulation protocol is shown having 2 second on (N) 2214, within a total time P of 10 seconds 2212, thus leaving an 8 second off time (P-N), and this is repeated to last for 60 minutes (D) with BBW at 20 Hz (F=1/T) of balanced symmetric biphasic waveforms.

The waveforms are shown to have the following. A pulse width of 0.2 ms (W), pulse amplitude of 0.5 mA (A) 2218, 0.1 ms (IP) 2220, ID 2222, and T=50 ms 2224. The waveform can be realized using 0.1 μs resolution for W, IP, T, while using slower (e.g., processor generated clocks) at ms timescales for N, P, and a slower clock at 1 second for D.

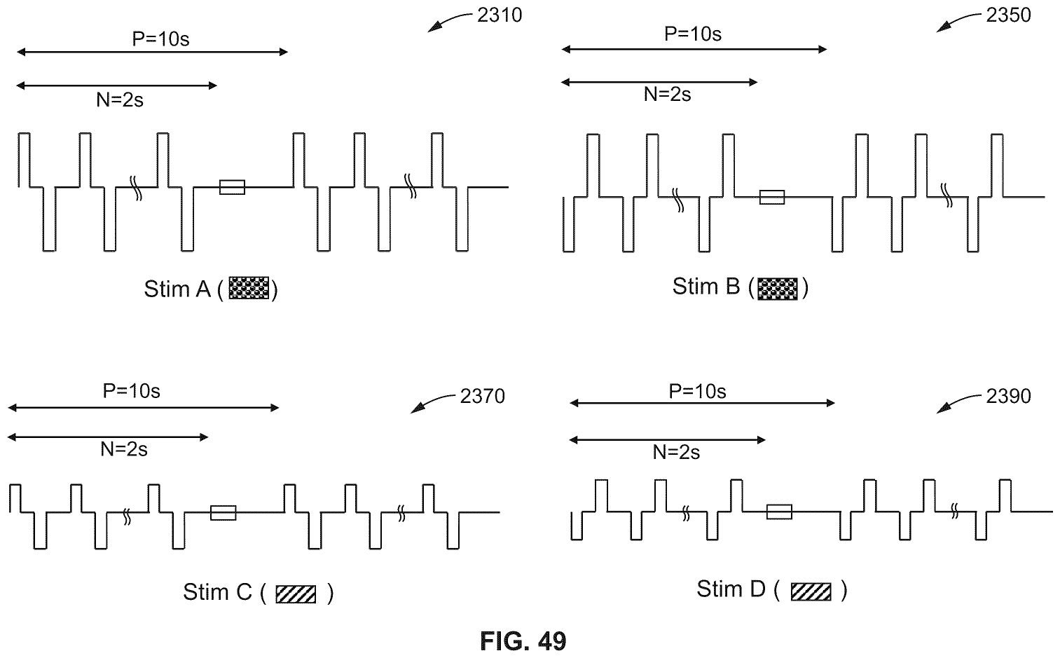

FIG. 49 illustrates an example embodiment 2350 of four different example stimulation protocols. Each electrode can be programmed as cathode or anode polarity in bipolar configuration mode or as cathode or anode as monopolar mode and each electrode can be assigned with a specific stimulation protocol. Furthermore, it is feasible to further support symmetric and asymmetric biphasic waveform with interphasic delay (IP) in either bipolar and/or monopolar configurations.

The stimulation specifications of the CDC hardware are as follows. The current passing through the stereotactic electrode array unit ranges from 0.1 mA to 50 mA, with voltage across the stereotactic electrode arrays ranging from 0.1 V to 30 V. The monophasic or biphasic current/voltage pulse may be delivered in stimulation protocols, including: (1) traditional uniform pulse train, (2) pulse train with varying pulse arrival time, (3) burst pulse train—each burst consists of both ON time and OFF time segments and this burst is periodically generated at a so called pulse train frequency (f_{B}); while each ON time segment is further made of another high frequency pulse train at (f_{H}), and (4) biomimetic waveform. The pulse train frequency (f_{B}) may be from 1 Hz to 100 Hz, preferably from about 2 Hz to 60 Hz, while the high frequency pulse train at (f_{H}) is adjusted based on the selected (f_{B}). The range of (f_{H}) is from 1 kHz to 10 kHz. The pulse width may be from 50 to 2000 microseconds. The preferred generated electric field can be directed parallel to the axis of the target nerve. Accordingly, the stimulator device of the PNPA system can support suitable waveform and optimal parameters including frequency, current, voltage, pulse width, and so forth. These waveforms are utilized in a stimulation montage described in the usage example of Section 7.3.2.1.

### 7.3.2 Stereotactic Stimulation Electrode Arrays (SSEA)

In one embodiment of the PNPA system of this disclosure, the stereotactic stimulation electrode arrays deliver the electric pulse generated by the stimulator device to the desired target area via the electrode arrays. The electrode array could be either an invasive or non-invasive one.

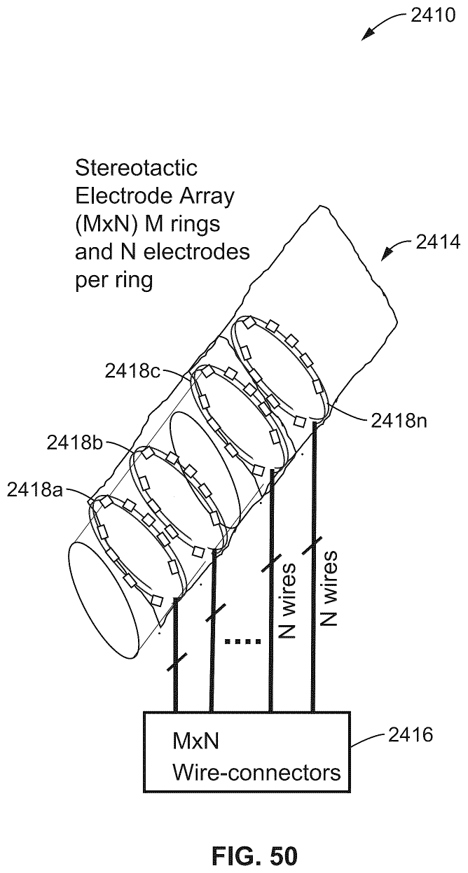

FIG. 50 illustrates an example embodiment 2410 of the Stereotactic Stimulation Electrode Array (SSEA), configured for delivering electrical stimulation non-invasively. The electrode current is set according to a specific optimal montage for the stereotactic electrode array, and the currents can be focally directed to the desired nerve/area in a stereotactic manner. The parameters resulting in the distribution of the electric currents vary according to the anatomic location of the targeted nerve. Specifically, the stereotactic stimulation electrode arrays consist of multiple ring structure stereotactic electrode arrays 2418a, 2418b, 2418c, through to 2418n. Each ring consisted of N independent electrodes and the SSEA consisted of M rings, which could deliver in total of M×N independent signals transcutaneously for stereotactic stimulations connected to the stimulator device 2416.

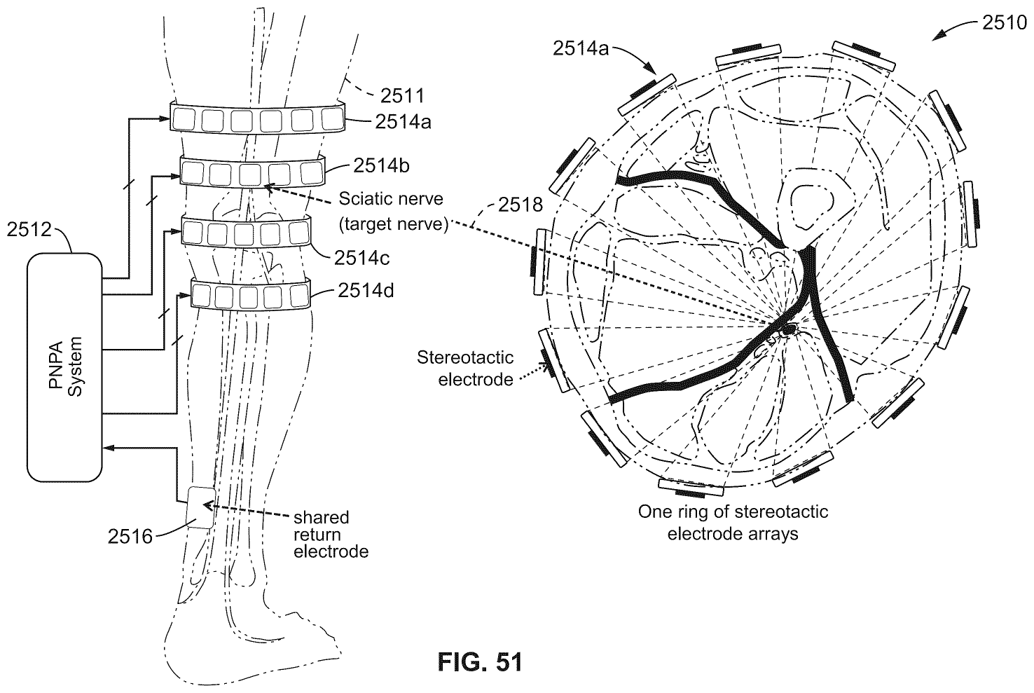

FIG. 51 illustrates an embodiment of 2510 of a multiple ring structure stereotactic electrode arrays wrapping around an extremity, in this case a patient's leg, with the figure showing in sciatic nerve 2518 in relation to other structures in both the plan view and the cross-section view. Each ring (band) is configured with an electrically conducting media sandwiched by electrodes and a non-conductive material layer for the band.

In the exemplary embodiment of sciatic-tibial nerve neuromodulation shown in the figure stereotactic stimulation electric arrays 2514a, 2514b, 2514c and 2514d are shown along with a shared return electrode 2516 on the appendage. The drawing on the right side shows a cross section of the appendage in which only one ring is seen. By way of example, and not limitation, this specific multiple ring structure of stereotactic arrays is specifically configured to target the sciatic-tibial nerve. The stereotactic electrodes are mounted in multiple thigh bands (rings) which, for example, can be wrapped around the leg in the region of the knee joint. The conducting media provides electrical conduction between the PNPA system 2512 and the electrode arrays and the patients tissue 2511 and thus prevents direct contact of the patient tissues and the stereotactic electrode array.

The electrodes in the array are separated by an insulating material. As an illustration example, the targeted sciatic and tibial nerve, located at the posterior position of the knee, can be selectively stimulated using the stereotactic electrode array being wrapped around the knee region. The electrode arrays are driven by an optimal current montage obtained by the optimization method as described in the next section. In one embodiment, the currents specified by the optimal montage can be at different frequencies, such that the Temporal Interference Stimulation (TIS) can be achieved. The electrical current stimuli montage generated by the stimulator device are applied to the stereotactic electrode arrays.

### 7.3.2.1. Targeting Sciatic Nerve Using a Montage by PNPA System

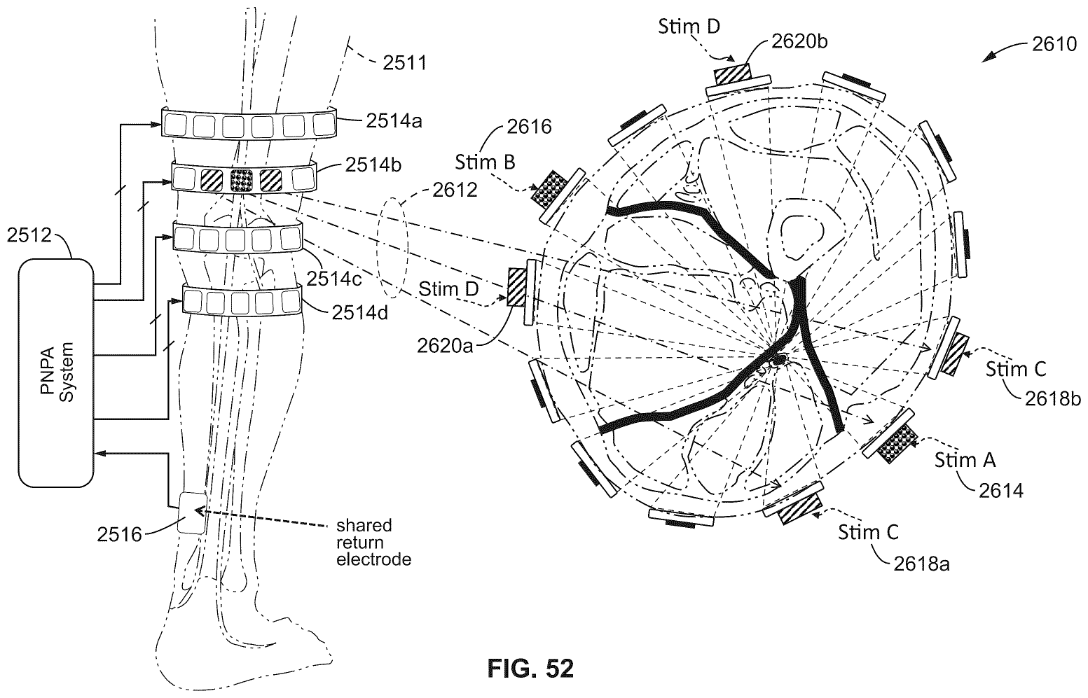

FIG. 52 illustrates an example embodiment 2610 of utilizing the multiple electrode ring structure of FIG. 51 connected to the PNPA system previously described, and using this interoperative combination in an optimization method with the GUI, the stimulator device and feedback monitor device. The optimal montage of current obtained by the optimization method is utilized to guide the GUI to drive the PNPA hardware/firmware system and accordingly the correct stimuli waveforms are applied to the stereotactic electrodes identified by the montage. As a result, the target nerve is focally stimulated by the desired stimulation protocols via transcutaneous approach.

The figure depicts the stimulation montage to target the sciatic nerve by properly activating a ring of the SSEA with stimulation waveforms of FIG. 49. By way of example and not limitation, here it is shown that there are six of the electrode arrays being activated 2614, 2616, 2618a, 2618b, 2620a, 2620b and 2622. In the Example the electrode arrays are activated 2612, a portion of which are most clearly seen on the left side portion of the figure, with arrows projecting to the figure on the right. It should be noted that there is an opposite polarity being applied by the system between Stim A and Stim B, as well as between Stim C and Stim D. In other words, anodic pulse is applied at Stim A, then the counter cathodic pulse is applied at Stim B. It is clear the summation of the currents is zero.

### 7.3.2.2. Optimal Montages for Stereotactic Electrode Array by Optimization Method

A novel technique has been developed utilizing established convex optimization theory to optimize the stimulation montage of stereotactic array electrodes. This technique is intended to target specific single peripheral nerves or a combination of multiple peripheral nerves, such as the sciatic, tibial, peroneal, and sural nerves in the thigh, knee, calf, heel, foot, as well as the radial, median, and ulnar nerves in the arm. The approach considers the thigh, calf, heel, foot, and arm as a volume conduction model, incorporating various tissues with distinct electrical conductivity, including skin, fat, muscle, bone, and nerves. The activation of action potential at a specific axon location is determined by the second-order partial spatial derivative of the voltage at that location, using the Passive Cable Equation commonly employed in neuromodulation.

To optimize the stimulation montage, the target tissue (e.g., the thigh) is divided into n small voxels in a 3-dimensional space (x, y, z). The induced voltage field resulting from stimulation currents at the electrodes of the stereotactic array (also applicable to surface electrode arrays) is represented as v (x, y, z) for each voxel. The Hessian Operator, a 3×3 matrix operator, is then applied to the voltage field, yielding the second-order partial spatial derivatives in the 3D space. This process generates a “hessian-field” expressed as a column vector H_{9n×1}. The “hessian-field” exhibits quasi-static behavior when low-frequency stimulation is applied to thigh and arm tissues. It should be noted that the model is often simplified from three dimensions to one dimension, considering that the direction of the peripheral nerve (e.g., sciatic nerve) can align with a specific coordinate. Consequently, the column vector is further reduced to H_{n×1} in the one-dimensional model, representing the second derivative with respect to the specific coordinate.

The column vector S_{m×1} represents the stimulation current sources (montages) applied at the electrodes of the stereotactic array or surface electrode array. The corresponding “hessian-field” H_{9n×1} at the target tissue (e.g., a set of thigh voxels containing the sciatic nerve) can be obtained through the matrix operation H_{9n×1}=K_{9n×m}*S_{m×1}. Each entry of the lead field matrix K_{9n×m} (Note: H_{n×1}=K_{n×m}*S_{m×1} for the one-dimensional model) specifies the value of the “hessian-field” at each voxel of the thigh or arm, contributed by the stimulation montage currents at the electrode system. Calculating each column vector in K_{9n×m} involves employing the Finite Electromagnetic Field Model of the thigh or arm using software tools like COMSOL Multiphysics (COMSOL Inc., Burlington, MA, USA). It is essential to note that the column vector depends on the locations of the current source electrode (one at the stereotactic array) and the return electrode, typically placed at the leg or other body structures.

The convex optimization method utilizes a cost function designed to balance the stimulation focality and intensity at the nerve tissues. Focality is represented by the total energy at non-target tissue regions, denoted as ∥Kₙₜ*S∥² using the L2 norm. Here, Kₙₜ, a submatrix of K, corresponds to the stimulated current at non-target tissues. On the other hand, the intensity energy at the target regions is expressed as the matrix operation Hₜ^{T}*Kₜ*S, where Hₜ is a column vector specifying the desired intensity of the “hessian-field” at the target nerve tissue(s), Kₜ is the submatrix of the leading field matrix K that depends on the current at the target tissue(s), and S is the vector of the stimulation montage current. Consequently, the optimal stimulation montage currents, S_{m×1}, can be obtained by solving the following optimization problem based on the cost function of stimulation focality and intensity energy, while adhering to constraints, such as the upper limit of current at each current source and ensuring the algebraic sum of currents S_{m×1} at the skin over the thigh, calf, or arm equals zero.

S=arg min(∥W*Kₙₜ*S∥²−λHₜ^{T}*Kₜ*S)

To incorporate the contribution of target and non-target voxels, a weighting matrix W is introduced. The regulation parameter A determines the energy tradeoff between the stimulation focality and the intensity at the target nerves. When A is small, the emphasis is on intensity, while a larger value emphasizes stimulation focality.

In one embodiment, a multiple-ring stereotactic electrode array is configured to generate a voltage field that maximizes the “hessian-field” at the voxels of the target nerve, aligning with the nerve's anatomical direction. The “hessian-field” at the target voxels activates voltage-gated ion channels at the target nerves, leading to the generation and propagation of action potentials along the nerves. This mechanism is crucial for inducing desired neurophysiological responses, including neuro-modulatory and analgesic effects, as demonstrated in preclinical animal experiments.

### 7.3.3. Feedback Monitoring Device

In one embodiment of the PNPA system of this disclosure, the feedback monitoring device detects the anterograde and/or retrograde neurophysiological response of the target nerve and output signals to regulate and optimize the electric impulse. The Somatic and Autonomic Sensor Electrodes (SASE) are utilized to detect the neurophysiological response. The detected signal may be filtered and can be used as closed-loop feedback to the stimulator device.

In an exemplary embodiment of the feedback monitoring device, the nerve conduction velocity (NCV) signals and electromyography (EMG) signals are recorded from the somatic sensor electrodes. The NCV and EMG neurophysiological responses are processed by the feedback monitoring device and are used to adjust the stimulation signal, thus forming a closed-loop system. In another exemplary embodiment, the vital sign metrics including the heart rate and the blood pressure are measured via the autonomic sensor electrodes. The heart rate variability is analyzed by Fourier transform in the feedback monitoring device. Characteristic frequencies representing the sympathetic and the parasympathetic system are used to monitor the effect of the PNPA system. Additionally, a safety alarm system is designed to indicate any heart rate increase or decrease by more than 20% of the baseline heart rate.

For the present medical applications, the PNPA system may be applied to the patient's upper and lower limb to stimulate the desired nerve. The stereotactic arrays may be positioned on the body surfaces. In a preferred embodiment of the PNPA system of this disclosure, the nerve which neuropathic pain condition is attributed to, is selected as the target. The anatomical location of the target nerve may be obtained by existing available techniques, such as ultrasound, computed tomography, and magnetic resonance examination and the information regarding the location of the target nerve can be used to adjust the parameters in the stimulation delivery device.

### 8. Preliminary Results of Neuropathic Pain Experiments

### 8.1. Experiment Protocol

### 8.1.1. Validation of the Therapeutic Effects of PNS

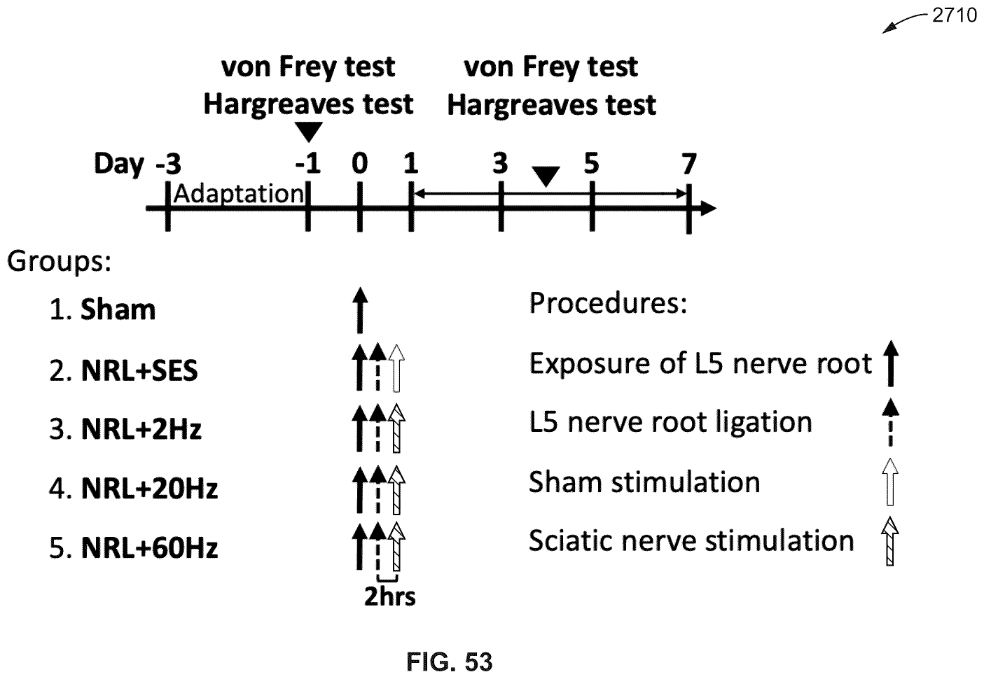

FIG. 53 illustrates an example of 5 different tests 2710 conducted to evaluate the effect of the neuromodulation in the peripheral nerve using sciatic nerve as an example. Five different sets of animal experiments, including neuromodulation with sciatic nerve stimulation (SNS) at three frequencies, were performed: Sham (exposure of L5 nerve root without ligation, N=10); NRL+Sham electrical stimulation (NRL+SES, N=10); NRL+2 Hz SNS (NRL+2 Hz, N=10); NRL+20 Hz SNS (NRL+20 Hz, N=10); and NRL+60 Hz SNS (NRL+60 Hz, N=10). SNS was performed ipsilateral to the ligated L5 nerve root at a location distal to the joint of L4, L5, and L6 nerve roots. The nerve was stimulated via hook electrode at frequencies of 2 Hz, 20 Hz, and 60 Hz with 0.2 ms biphasic square-wave pulses. In the NRL+SES group, Sham electrodes without electrical current were applied to the sciatic nerve. Starting three days before surgery, the rats were introduced to the testing environment daily to allow for acclimation. The rats were placed in the environment used for the mechanical and thermal tests for 30 minutes before the tests were performed. Before and 1, 3, 5, and 7 days after surgery, the plantar surface of both ipsilateral and contralateral hind paws was probed using electrical von Frey tips (BIO-EVF5, Bioseb, France) to measure the thresholds of mechanical touch sensitivity (von Frey test). The tip was gently applied upward onto the rat's middle plantar surface and force was slowly exerted until the rats withdrew, flicked, or licked their paw. The reading of the largest force (in grams) was automatically recorded. Each rat was tested five times separated by 15 s intervals. Hargreaves test was performed on preoperative day 1 and 1-, 3-, 5-, and 7-days post-surgery to evaluate thermal sensitivity using a Plantar Test Apparatus (Ugo Basile, Comerio, Italy). An infrared heat source (50 W) was adjusted so that naïve rats had withdrawal latencies of 9-12 s. The heat source was focused on the plantar surface of the ipsilateral and contralateral hind paw and the time taken to withdraw from the heat stimulus was recorded. Each hind paw was tested three times separated by intervals of 2 min.

### 8.1.2. Validation of Stereotactic Electric Nerve Stimulation

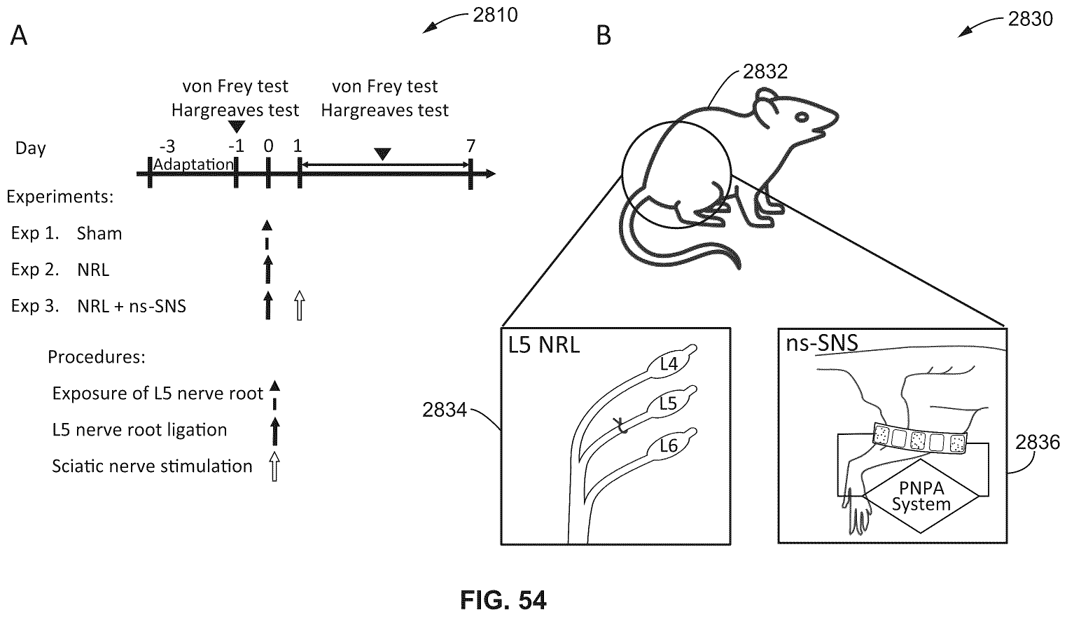

FIG. 54 is an illustration 2830 of evaluating the effect of non-invasive stereotactic neuromodulation using stereotactic stimulation electrode arrays (SSEA) in the peripheral nerve using sciatic nerve as an example. Three different sets of animal experiments were performed on the rats 2832. The Sham group (group 1) with the L5 nerve root exposed, but not ligated was used as the control. In the ns-SNS groups (groups 3), ns-SNS was performed on day 1 after NRL 2834 using the PNPA system with SSEA encircling 2836 the right thigh of the NRL rat to target the right sciatic nerve.

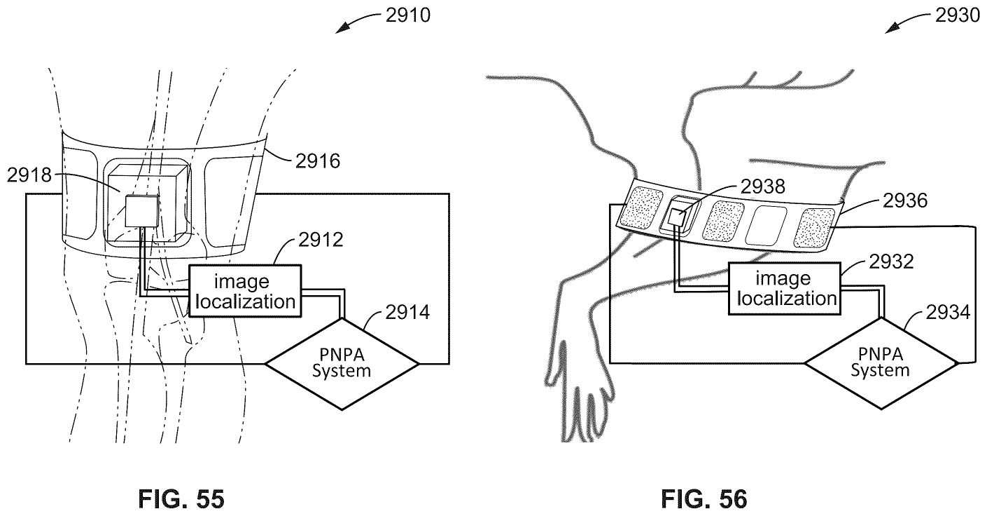

FIG. 55 and FIG. 56 illustrates 2910, 2930 demonstrating assess points of SSEA for ns-SNS, shown on a human and a rat. Specifically, two pairs of stereotactic electrodes were utilized in each case. In this exemplary embodiment, ultrasonic probe 2918, 2938, placed at the surface of thigh or arm or leg, was used to locate 2912, 2932 the target nerves, such as the sciatic nerve. Specifically, the direction and depth of the sciatic nerve was measured using ultrasound to tune the parameters of the electrodes connected to the PNPA system 2914, 2934.

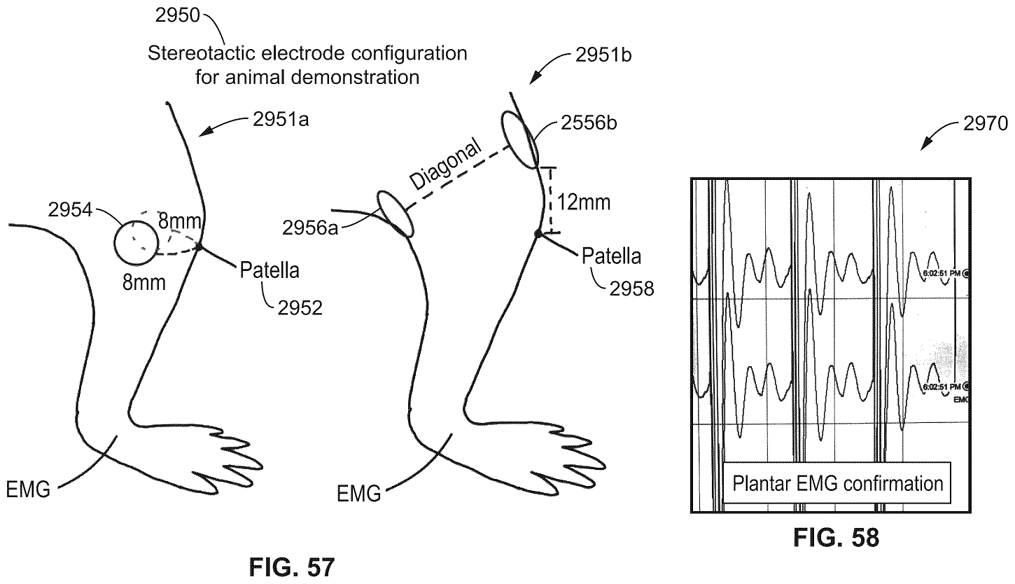

FIG. 57 illustrates 2950 additional details beyond FIG. 56, on electrode ring placement for the rat example. Two diagrams 2951 a, 2951 b are shown. The patella bone 2952 was identified by manual palpation as the body landmark to assist lead placement. In the first pair of electrodes, Lead #1 was placed at the same horizontal level to the patella, and 8 mm laterally 2954. Lead #2 was placed Opposing Lead #1, at the same horizontal level to the patella, located 8 mm medially. For the second pair of electrodes, Lead #1 was placed 2956a at the level 12 mm superior to the patella 2958, located at the anterior edge of the thigh and Lead #2 was placed 2956b opposing to Lead #1, located diagonally at the posterior edge of the thigh.

The stimulation parameters were as follows: Electrode pair 1: Two-second-long uniform biphasic pulse trains, separated by 8-s off intervals; Frequency: 20 Hz; Pulse width: 200-μs; Intensity: 4.0 mA (range: 1 to 10 mA). Electrode pair 2: Two-second-long uniform biphasic pulse trains, separated by 8-s off intervals; Frequency: 20 Hz; Pulse width: 200-μs; Intensity: 4.5 mA (range: 1 to 10 mA).

FIG. 58 illustrates an example 2970 of a Plantar EMG signal confirming delivery of nerve stimulation. The signal of plantar EMG was utilized as feedback return electrode in this experimental validation.

### 8.2. Experiment Evaluation Results

### 8.2.1. Animal Pain Behavior

To evaluate the effect of neuromodulation in the peripheral nerve using sciatic nerve as an example, we first evaluated mechanical (von Frey test) and thermal (Hargreaves test) pain sensitivity in L5 NRL rats. The Sham group with the L5 root exposed but not ligated was used as a control.

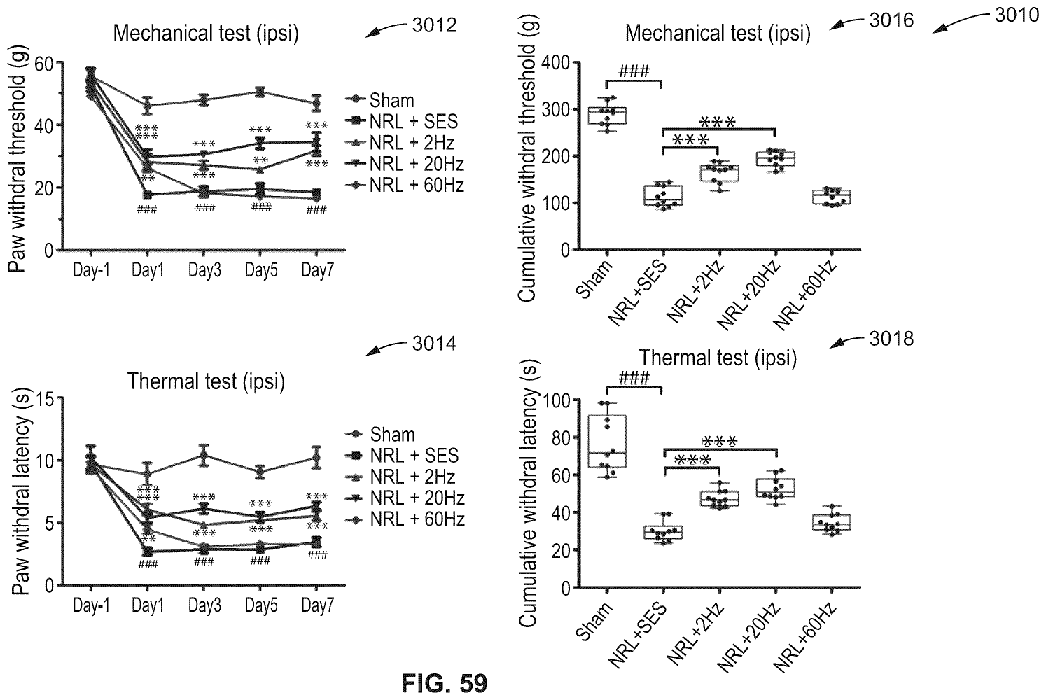

FIG. 59 illustrates testing results 3010, including a mechanical test 3012 of paw withdrawal threshold, a thermal test 3014 of paw withdrawal threshold, a mechanical test 3016 of cumulative paw withdrawal threshold, and a thermal test 3018 of paw withdrawal threshold.

As shown in the figure, compared to the Sham group, NRL+SES rats exhibited a significantly lower paw withdrawal threshold and decreased paw withdrawal latency from Post-Injury Day 1 (PID1) to PID7 on the ipsilateral side but not the contralateral side. To further investigate the therapeutic potential of SNS and the efficacy of different electrical frequencies, the pain behaviors of SNS rats following 2 Hz, 20 Hz, and 60 HZ stimulation were evaluated. On PID1, all frequencies of SNS increased the paw withdrawal thresholds and latencies compared to NRL+SES. On PID 3, 5, and 7, increased paw withdrawal thresholds and latencies compared to NRL+SES were observed for 2 Hz and 20 Hz SNS, but not 60 Hz SNS. The pain and thermal sensitivities of the contralateral paw were unchanged. The results showed that L5 NRL resulted in significantly decreased ipsilateral cumulative withdrawal thresholds and latencies and that the decreases were ameliorated by 2 Hz and 20 Hz SNS, but not 60 Hz SNS.

### 8.2.2. Histological and Biochemical Experiments

To evaluate the effect of neuromodulation in the cellular level and to investigate whether neuromodulation impacted the inflammatory response in the spinal cord, we performed western blot analysis and immunofluorescence staining.

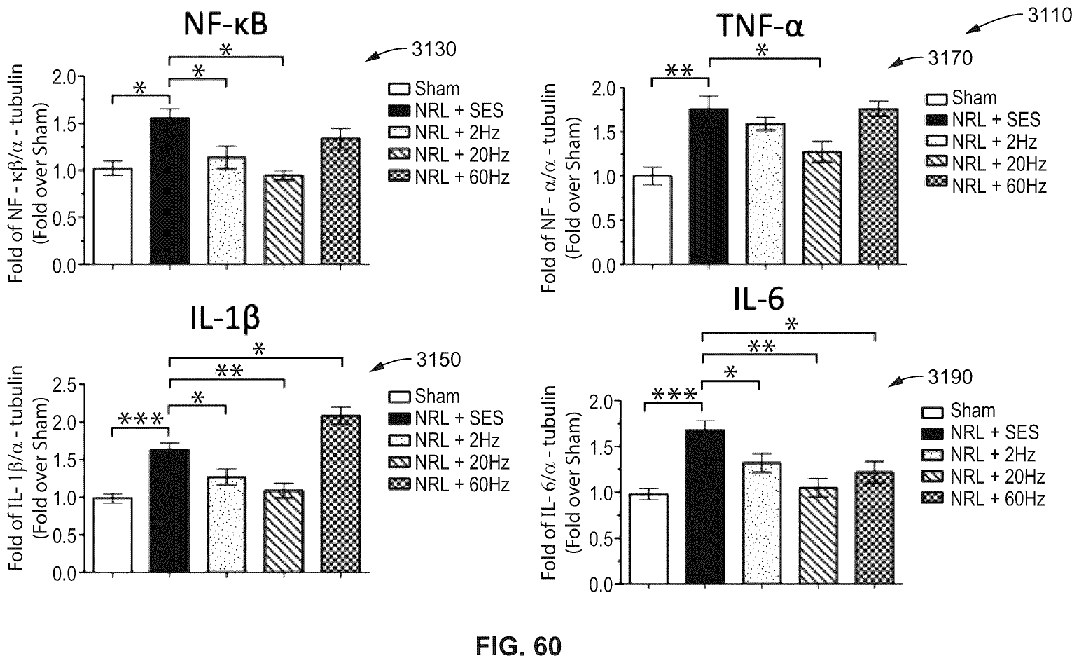

FIG. 60 illustrates testing of the expression 3110 of the inflammatory proteins including NF-κB 3130, TNF-α 3110, IL-1β 3150, and IL-6 3190 on PID7 were measured by western blotting. Seven days after nerve injury, the levels of spinal cord NF-κB, TNF-α, IL-1β, and IL-6 were significantly elevated by 1.6-fold, 1.8-fold, 1.6-fold, and 1.7-fold in the NRL+SES group compared to the Sham group, respectively. In the neuromodulation group, we observed that 20 Hz SNS reduced the expression of TNF-α, 2 Hz and 20 Hz SNS reduced the expression of NF-κB, and all frequencies of SNS reduced the expression of IL-1β 3 and IL-6.

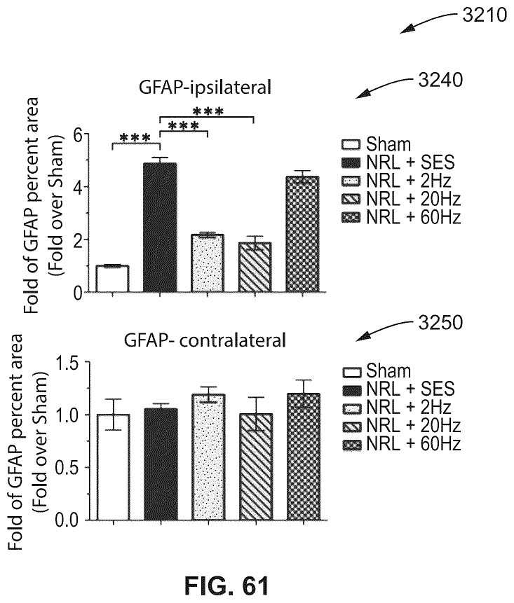

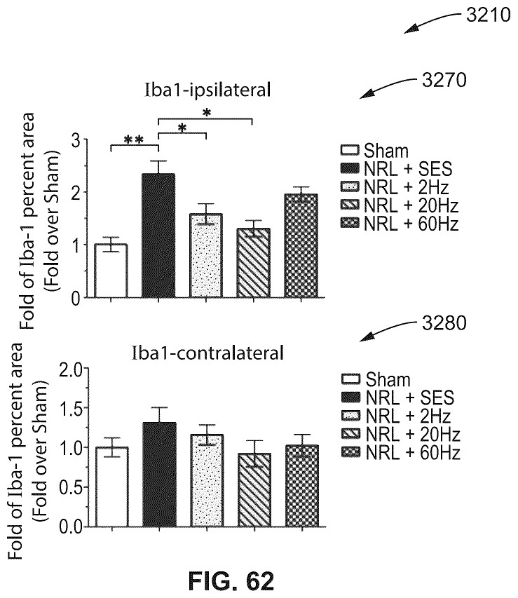

FIG. 61 and FIG. 62 illustrate tests on activation 3210 of the inflammatory cells in the spinal cord following SNS were measured by immunofluorescent staining. The amount of activated spinal cord astrocytes and microglia were measured on PID7.

In FIG. 61 the quantification of the ipsilateral GFAP 3240 signal in the L5 spinal cord revealed a 4.9-fold increase of GFAP area in the NRL+SES group compared to the Sham group. Notably, the increased ipsilateral signal was ameliorated by 2 Hz and 20 Hz SNS, but not 60 Hz SNS. The contralateral GFAP 3250 signal was not affected by L5 NRL and SNS.

In FIG. 62 the Iba1 immunofluorescence revealed a 2.3-fold increase of ipsilateral Iba1 3270 area in the NRL+SES group compared to the Sham group. The increase in ipsilateral Iba1 signal was mitigated by 2 Hz and 20 Hz SNS, but not 60 Hz SNS. The contralateral Iba1 (biomarker of microglia) 3280 signal was not affected by L5 NRL and SNS. Together, the histological and biochemical experiments demonstrated that neuromodulation with SNS was able to alleviate the neuroinflammatory responses in NP.

### 8.2.3. Validation of Electric Stimulation Using Stereotactic Stimulation Electrode Arrays (SSEA)

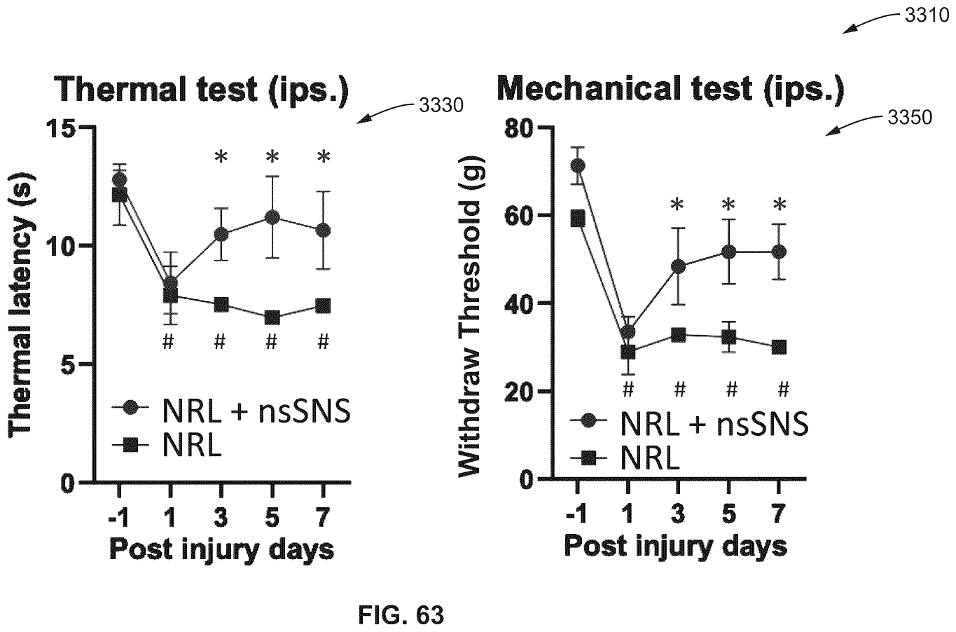

FIG. 63 illustrates experimental results 3310 of pain behavior in animals receiving ns-SNS. Thermal latency is shown 3330 as well as a mechanical test of withdrawal threshold 3350. The paw withdrawal latency and threshold on the ipsilateral side following to NRL was significantly decreased compared to baseline (day −1). Implementation of ns-SNS significantly increased the paw withdraw threshold and latency. This result demonstrated that ns-SNS with SSEA decreased the neuropathic pain phenotypes including thermal hypersensitivity and mechanical allodynia following L5 nerve injury. The elevated activation of spinal cord inflammatory cells following nerve injury was also reduced after the application of stereotactic electric nerve stimulation. This experiment validated the application of non-invasive stereotactic electric nerve stimulation.

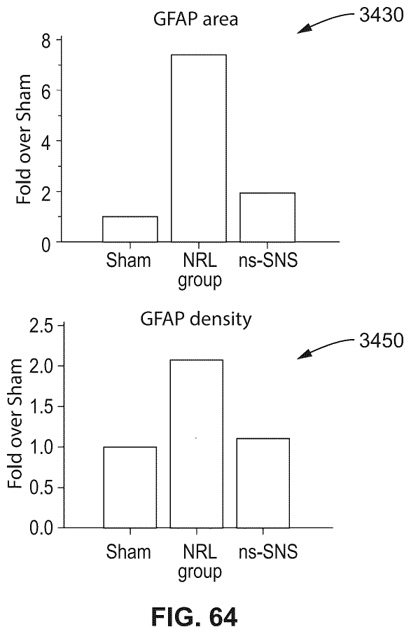

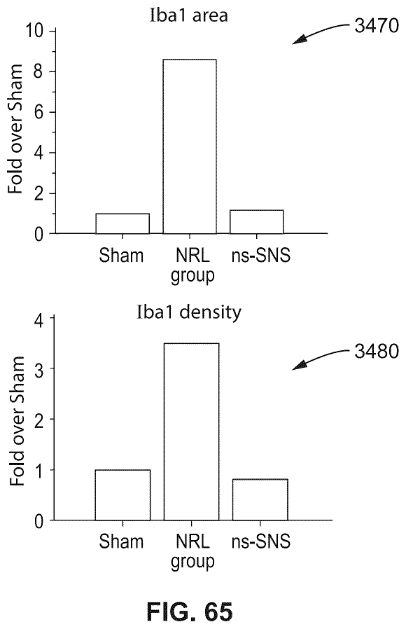

FIG. 64 and FIG. 65 illustrate activation of the inflammatory cells in the spinal cord following SNS were measured by immunofluorescent staining. The bar charts show GFAP area 3430, GFAP density 3450, Iba1 area 3470 and Iba1 density 3480. The amount of activated spinal cord astrocytes and microglia were measured on PID7. In the NRL group, the ipsilateral GFAP signal significantly increased compared to the Sham group. Notably, the increased ipsilateral signal was ameliorated by 2 ns-SNS. The Iba1 immunofluorescence staining showed increase of ipsilateral Iba1 area and density in the NRL group compared to the Sham group. The increase in ipsilateral Iba1 signal was mitigated by ns-SNS. Together, the histological and biochemical experiments demonstrated that ns-SNS with SSEA alleviates the neuroinflammatory response of spinal cord astrocytic gliosis following nerve injury of L5 NRL.

### 9. General Scope of the Embodiments

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple implementations of the technology which include, but are not limited to, the following:

A system of electrical peripheral nerve neuromodulation for alleviating painful neuropathic conditions, comprising: (a) a stimulator circuit; (b) stereotactic stimulation electrode arrays (SSEA), configured for securement at a first skin position of a patient; (c) somatic and autonomic sensor electrodes (SASE) configured for securement at a second skin position on the patient; (d) a feedback monitoring circuit configured for receiving input from said SASE for receiving electromyography (EMG) and nerve conduction velocity (NCV); (e) communication circuitry, wired and/or wireless, which provides interconnection between said stimulator circuit, SSEA, SASE and said feedback monitoring circuitry; and (f) wherein said stimulator circuit is configured for receiving control parameters for a stimulation protocol from an external control device, from which it performs assembling basic building waveforms, counters, and timers which when combined are directed to a current/voltage driver hardware (CDC), to generate the stimulation protocol.

A method of generating and delivering an electric field to the target a peripheral nerve, comprising: (a) generating an electric field which is customized for an anatomical location of a target nerve of interest, which may be superficial or deep in the body, and delivered thereto; (b) wherein said delivered electric field elicits action potentials from the target nerve; (c) wherein the elicited action potentials travel anterogradely to the nerve terminus and retrogradely to the central nervous system; and (d) wherein said retrograde action potential exerts neuromodulation effects in the central nervous system.

A peripheral nerve neuromodulation system comprising: (a) a stimulator device configured to generate an electric pulse sufficient to produce an electric field and/or current at the target nerve to induce an anterograde and/or retrograde membrane potential propagation in the targeted peripheral nerve; (b) a stimulation delivery device configured to deliver the electric pulse protocols at the target nerves at an upper and lower extremity through electrode array; (c) wherein said stimulation delivery device is configured to non-invasively deliver the electric pulse protocols at the target nerves by employing a ring structure stereotactic electrode array configured to surround the extremity; and (d) wherein non-invasively deliver of the electric pulse protocols at the target nerves is performed using electric pulse montages configured for selectively focusing on the target.

A stimulation delivery apparatus, comprising: (a) a ring structure stereotactic electrode array configured to non-invasively deliver electric pulse protocols at target nerves; (b) wherein said ring structure is configured for being positioned around an area of an arm, a thigh, or a leg; (c) wherein montages of electric pulses within a pulse protocol are applied between electrodes at different radial positions around said arm to selectively focus on the radial, median, and ulnar nerves at the elbow and arm, or on the sciatic, tibial, sural, peroneal, and sural nerves at the thigh and leg.

An electric stimulation feedback monitoring apparatus, comprising: (a) at least one somatic and autonomic sensor electrode (SASE); (b) wherein said at least one somatic and autonomic sensor electrode is combined with a system for generating electrical stimulation through a stereotactic stimulation electrode array (SSEA); and (c) wherein somatic and autonomic sensor electrode is configured for detecting anterograde and/or retrograde neurophysiological responses to generation of said electrical stimulation of a target nerve.

A method of electrical peripheral nerve neuromodulation which will alleviate painful neuropathic conditions via following mechanisms:

A method and device to generate and deliver an electric field to target a peripheral nerve.

A peripheral nerve neuromodulation system.

A stimulator apparatus configured to generate an electric pulse sufficient to produce an electric field and/or current at the target nerve to induce an anterograde and/or retrograde membrane potential propagation in the targeted peripheral nerve.

A stimulation delivery apparatus configured to either invasively or non-invasively deliver the electric pulse protocols at the target nerves at upper and lower extremity via electrodes or electrode array.

A stimulation delivery apparatus configured to non-invasively deliver the electric pulse protocols at the target nerves by employing the ring structure stereotactic electrode array around the thigh and other parts of lower extremity. With specifically optimal montages derived by the optimization method, the montages are capable of selectively focusing on the sciatic, tibial, and sural nerves.

A stimulation delivery apparatus configured to non-invasively deliver the electric pulse protocols at the target nerves by employing the surface electrode array (as shown in FIG. 6) on the thigh and other parts of lower extremity. With specifically optimal montages derived by the optimization method, the montages are capable of selectively focusing on the sciatic, tibial, and sural nerves.

A stimulation delivery apparatus configured to invasively deliver the electric pulse protocols at the target nerves by employing the cuff electrode on the thigh and other parts of lower extremity such that stimulation can specifically target at the sciatic, tibial, and sural nerves.

A stimulation delivery device configured to non-invasively deliver the electric pulse protocols at the target nerves of by employing the ring structure stereotactic electrode array around the elbow and arm. With specifically optimal montages derived by the optimization method, the montages are capable of selectively focusing on the radial, median, and ulnar nerves at the elbow and arm.

The apparatus or method or system of any preceding implementation, wherein neuromodulation effects in the central nervous system comprise modulation of spinal neuronal activity, modulation of neuroinflammation, and modulation of descending pain inhibition.

The apparatus or method or system of any preceding implementation, wherein neuromodulation effects in the peripheral nervous system comprise: decrease of neuronal activity of spinal dorsal horn pain transmitting neurons, decrease of dorsal root ganglia, spinal cord, and brain neuroinflammation, decrease of microglial and macrophage hyperactivation, decreased of satellite glia and astrocyte gliosis, and increased neuronal activities of descending pain inhibition pathway neurons.

The apparatus or method or system of any preceding implementation, wherein said neuromodulation effects in the peripheral nervous system addresses diseases of neuropathic conditions comprising: alcoholic neuropathy, diabetic neuropathy, post-amputation, chemotherapy-induced neuropathic pain, chemotherapy-induced cytopenia, multiple sclerosis, nerve or spinal cord compression from generation, herniated discs, arthritis, trauma, or neoplasm in the spine, and Herpes infection.

The apparatus or method or system of any preceding implementation, wherein the nerve associated with the neuropathic pain condition is attributed to, is selected as the target including the sciatic, tibial, and radial nerve.

The apparatus or method or system of any preceding implementation, wherein the nerve associated with the neuropathic pain condition is attributed to a cytopenia condition after chemotherapy is selected as the target nerve in the sciatic, tibial, peroneal, sural, and/or radial nerve.

The apparatus or method or system of any preceding implementation, wherein target nerves comprise sciatic, tibial, peroneal, sural, radial, median, and ulnar nerves.

The apparatus or method or system of any preceding implementation, further comprising an optimization method configured to generate an optimal montage of stimulation current having a selective focus on the target nerve.

The apparatus or method or system of any preceding implementation, wherein said target nerve is comprising sciatic, tibial, peroneal, and/or sural nerves in the thigh, knee, calf, heel, and foot.

The apparatus or method or system of any preceding implementation, wherein after stimulation, the nerve activates the proper molecular signal pathways along spinothalamic axis responding to pain treatment.

The apparatus or method or system of any preceding implementation, wherein after stimulation, the nerve activates the proper molecular signal pathways inside bone marrow, which respond to hematopoiesis after chemotherapy.

The apparatus or method or system of any preceding implementation, further comprising an optimization method to provide an optimal montage of stimulation current which is selectively focused on the target nerve.

The apparatus or method or system of any preceding implementation, wherein said ring structure stereotactic electrode array is configured to support temporal interference stimulation (TIS).

The apparatus or method or system of any preceding implementation, wherein said ring structure stereotactic electrode array is also configured for supporting ultrasound stimulation with an array of ultrasound transducers.

The apparatus or method or system of any preceding implementation, wherein each of the pulses has a duration from 20 microseconds to about 2000 microseconds.

The apparatus or method or system of any preceding implementation, wherein each of the pulses has a current passing through the SSEA from 0.1 mA to 50 mA.

The apparatus or method or system of any preceding implementation, wherein each of the pulses of said electrical stimulation of a target nerve have a voltage across the SSEA from 0.1 V to 30 V.

The apparatus or method or system of any preceding implementation, wherein each of the pulses of said electrical stimulation of a target nerve are delivered in monophasic or biphasic modes of a burst pulse train, tonic currents, sinusoidal, and/or combinations of modes.

The apparatus or method or system of any preceding implementation, wherein the pulses of said electrical stimulation have a frequency from 1 Hz to 100 Hz.

The apparatus or method or system of any preceding implementation, wherein distributed currents in the electrodes of the SSEA directs a generated electric field to the desired nerve/area in a stereotactic method using anatomical parameters.

The apparatus or method or system of any preceding implementation, wherein said anatomical parameters used in the distribution of the electric currents vary according to the information of the anatomic location of the target nerve.

The apparatus or method or system of any preceding implementation, wherein said feedback signals are utilized to direct the parameters in the device as closed-loop feedback.

The apparatus or method or system of any preceding implementation, wherein said feedback signals used in the closed-loop feedback include nerve conduction velocity (NCV) signals, electromyography (EMG) signals, heart rate, the blood pressure as neurophysiological responses; and wherein said feedback signals are used to adjust generated stimulation signals from said SSEA, and allows said SASE to monitor autonomic response following the application of a stimulation through said SSEA.

The apparatus or method or system of any preceding implementation, in which the peripheral nerve is in the leg or the arm.

The apparatus or method or system of any preceding implementation, wherein the configuration of the electric field generated is customized according to the nerve of interest, which may be superficial or deep in the human body.

The apparatus or method or system of any preceding implementation, wherein the generated electric field is delivered to the region of interest (the anatomical location of the target nerve).

The apparatus or method or system of any preceding implementation, wherein the delivered electric field elicits action potentials of the target nerve.

The apparatus or method or system of any preceding implementation, wherein the elicited action potentials travel anterogradely to the nerve terminus and retrogradely to the central nervous system.

The apparatus or method or system of any preceding implementation, wherein the retrograde action potential exerts neuromodulation effects in the central nervous system.

The apparatus or method or system of any preceding implementation, wherein the neuromodulation effects in the central nervous system include, but are not limited to, modulation of spinal neuronal activity, modulation of neuroinflammation, and modulation of descending pain inhibition.

The apparatus or method or system of any preceding implementation, wherein the neuromodulation effects in the peripheral nervous system include, but are not limited to, decrease of neuronal activity of spinal dorsal horn pain transmitting neurons with reduced glutamate release and decreased c-fos activity, decrease of dorsal root ganglia neuroinflammation with reduced GFAP and Iba1 expression decrease of spinal cord neuroinflammation with reduced GFAP and Iba1 expression, and increased neuronal activities of descending pain inhibition pathway neurons with increased serotonin release in the rostral ventral medulla region and increased endorphin release in the periaqueductal gray matter.

The apparatus or method or system of any preceding implementation, wherein the electrodes include a cuff electrode, such as in the case of minimally invasive surgery being utilized for addressing chronic neuropathy pain.

The apparatus or method or system of any preceding implementation, wherein the target nerves include, but are not limited to the sciatic, tibial, sural, radial, median, and ulnar nerves.

The apparatus or method or system of any preceding implementation, wherein an optimization method can provide an optimal montage of stimulation current such that a selective focus on the nerve (i.e., sciatic, tibial, and sural nerves in the thigh, knee, calf, heel, and foot) is performed.

The apparatus or method or system of any preceding implementation, wherein after stimulation, the nerve activates the proper molecular signal pathways along spinothalamic axis responding to pain treatment.

The apparatus or method or system of any preceding implementation, wherein after stimulation, the nerve activates the proper molecular signal pathways inside bone marrow, which respond to hematopoiesis after chemotherapy.

The apparatus or method or system of any preceding implementation, wherein an optimization method can provide an optimal montage of stimulation current such that a selective focus on the nerve, such as radial nerve in the arm, is performed.

The apparatus or method or system of any preceding implementation, wherein the ring structure stereotactic electrode array can be arranged to support Temporal Interference Stimulation (TIS).

The apparatus or method or system of any preceding implementation, wherein the ring structure stereotactic electrode array can be arranged to support ultrasound stimulation with array of ultrasound transducers.

The apparatus or method or system of any preceding implementation, wherein a feedback monitoring device, configured to detect the anterograde and/or retrograde neurophysiological response of the target nerve.

The apparatus or method or system of any preceding implementation, wherein each of the pulses has a duration from about 20 microseconds to about 2000 microseconds.

The apparatus or method or system of any preceding implementation, wherein each of the pulses has a current passing through the stereotactic electrode array unit from about 0.1 mA to about 50 mA.

The apparatus or method or system of any preceding implementation, wherein each of the pulses has a voltage across the stereotactic electrode array unit from about 0.1 V to about 30 V.

The apparatus or method or system of any preceding implementation, wherein each of the pulses may be delivered in monophasic or biphasic modes of burst pulse train, tonic currents, sinusoidal, and combinations of modes.

The apparatus or method or system of any preceding implementation, wherein the pulses have a frequency from about 1 Hz to about 100 Hz.

The apparatus or method or system of any preceding implementation, wherein the distributed currents in the electrodes of the stereotactic electrode arrays directs the generated electric field to the desired nerve/area in a stereotactic method using anatomical parameters.

The apparatus or method or system of any preceding implementation, wherein parameters used in the distribution of the electric currents vary according to the information of the anatomic location of the target nerve.

The apparatus or method or system of any preceding implementation, wherein the feedback signals are used to direct the parameters in the device of implementation 2-1 as closed-loop feedback.

The apparatus or method or system of any preceding implementation, wherein the feedback signals used in the closed-loop feedback include the nerve conduction velocity (NCV) signals, electromyography (EMG) signals, heart rate, the blood pressure as neurophysiological responses. The feedback signals are used to adjust the generated stimulation signal in the device of implementation 2-3 and monitor the autonomic response following the application of the stimulation using the device of implementation 2-3.

The apparatus or method or system of any preceding implementation, wherein the at least one of the disease or the neuropathic condition includes alcoholic neuropathy, diabetic neuropathy, post-amputation, chemotherapy-induced neuropathic pain, chemotherapy-induced cytopenia, multiple sclerosis, nerve or spinal cord compression from generation, herniated discs, arthritis, trauma, or neoplasm in the spine, Herpes infection are applied.

The apparatus or method or system of any preceding implementation, wherein the nerve which neuropathic pain condition is attributed to, is selected as the target including the sciatic, tibial, and radial nerve.

The apparatus or method or system of any preceding implementation, wherein the nerve which cytopenia condition after chemotherapy is attributed to, is selected as the target including the sciatic, tibial, and radial nerve.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has . . . a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10⁰, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A system of electrical peripheral nerve neuromodulation for alleviating painful neuropathic conditions, comprising:
a stimulator circuit;
stereotactic stimulation electrode arrays (SSEA), configured for securement at a first skin position of a patient;
somatic and autonomic sensor electrodes (SASE) configured for securement at a second skin position on the patient;
a feedback monitoring circuit configured for receiving input from said SASE for receiving electromyography (EMG) and nerve conduction velocity (NCV);
communication circuitry, wired and/or wireless, which provides interconnection between said stimulator circuit, SSEA, SASE and said feedback monitoring circuitry; and
wherein said stimulator circuit is configured for receiving control parameters for a stimulation protocol from an external control device, from which it performs assembling basic building waveforms, counters, and timers which when combined are directed to a current/voltage driver hardware (CDC), to generate the stimulation protocol.

2. A method of generating and delivering an electric field to the target a peripheral nerve, comprising:
generating an electric field which is customized for an anatomical location of a target nerve of interest, which may be superficial or deep in the body, and delivered thereto;
wherein said delivered electric field elicits action potentials from the target nerve;
wherein the elicited action potentials travel anterogradely to the nerve terminus and retrogradely to the central nervous system; and
wherein said retrograde action potential exerts neuromodulation effects in the central nervous system.

3. The method of claim 2, wherein neuromodulation effects in the central nervous system comprise modulation of spinal neuronal activity, modulation of neuroinflammation, and modulation of descending pain inhibition.

4. The method of claim 2, wherein neuromodulation effects in the peripheral nervous system comprise: decrease of neuronal activity of spinal dorsal horn pain transmitting neurons, decrease of dorsal root ganglia, spinal cord, and brain neuroinflammation, decrease of microglial and macrophage hyperactivation, decreased satellite glia and astrocyte gliosis, and increased neuronal activities of descending pain inhibition pathway neurons.

5. The method of claim 2, wherein said neuromodulation effects in the peripheral nervous system addresses diseases of neuropathic conditions comprising: alcoholic neuropathy, diabetic neuropathy, post-amputation, chemotherapy-induced neuropathic pain, chemotherapy-induced cytopenia, multiple sclerosis, nerve or spinal cord compression from generation, herniated discs, arthritis, trauma, or neoplasm in the spine, and Herpes infection.

6. The method of claim 5, wherein the nerve associated with the neuropathic pain condition is selected as the target treatment area including the sciatic, tibial, peroneal, sural, and radial nerve.

7. The method of claim 5, wherein the nerve associated with the neuropathic pain condition is attributed to a cytopenia condition after chemotherapy and is selected as the target nerve in the sciatic, tibial, peroneal, sural, and/or radial nerve.

8. The method of claim 2, wherein target nerves comprise sciatic, tibial, peroneal, sural, radial, median, and ulnar nerves.

9. A peripheral nerve neuromodulation system comprising:
a stimulator device configured to generate an electric pulse sufficient to produce an electric field and/or current at the target nerve to induce an anterograde and/or retrograde membrane potential propagation in the targeted peripheral nerve;
a stimulation delivery device configured to deliver the electric pulse protocols at the target nerves at an upper and lower extremity through an electrode array;
wherein said stimulation delivery device is configured to non-invasively deliver the electric pulse protocols at the target nerves by employing a ring structure stereotactic electrode array configured to surround the extremity; and
wherein non-invasive delivery of the electric pulse protocols at the target nerves is performed using electric pulse montages configured for selectively focusing on the target.

10. The system of claim 9, further comprising an optimization method configured to generate an optimal montage of stimulation current having a selective focus on the target nerve.

11. The system of claim 9, wherein said target nerve is comprising sciatic, tibial, peroneal, and/or sural nerves in the thigh, knee, calf, heel, and foot.

12. The system of claim 9, wherein after stimulation, the nerve activates the proper molecular signal pathways along spinothalamic axis responding to pain treatment.

13. The system of claim 12, wherein after stimulation, the nerve activates the proper molecular signal pathways inside bone marrow, which responds to hematopoiesis after chemotherapy.

14. A stimulation delivery apparatus, comprising:
a ring structure stereotactic electrode array configured to non-invasively deliver electric pulse protocols at target nerves;
wherein said ring structure is configured for being positioned around an area of an arm, a thigh, a leg;
wherein montages of electric pulses within a pulse protocol are applied between electrodes at different radial positions around said arm to selectively focus on the radial, median, and ulnar nerves at the elbow and/or arm or on the sciatic tibial, sural, peroneal, and sural nerves at the thigh and leg.

15. The apparatus of claim 14, further comprising an optimization method to provide an optimal montage of stimulation current which is selectively focused on the target nerve.

16. The apparatus of claim 14, wherein said ring structure stereotactic electrode array is configured to support temporal interference stimulation (TIS).

17. The apparatus of claim 14, wherein said ring structure stereotactic electrode array is also configured for supporting ultrasound stimulation with an array of ultrasound transducers.

18. An electric stimulation feedback monitoring apparatus, comprising:
at least one somatic and autonomic sensor electrode (SASE);
wherein said at least one somatic and autonomic sensor electrode is combined with a system for generating electrical stimulation through a stereotactic stimulation electrode array (SSEA); and
wherein somatic and autonomic sensor electrode is configured for detecting anterograde and/or retrograde neurophysiological responses to the generation of said electrical stimulation of a target nerve.

19. The apparatus of claim 18, wherein each of the pulses has a duration from 20 microseconds to about 2000 microseconds.

20. The apparatus of claim 18, wherein each of the pulses has a current passing through the SSEA from 0.1 mA to 50 mA.

21. The apparatus of claim 18, wherein each of the pulses of said electrical stimulation of a target nerve have a voltage across the SSEA from 0.1 V to 30 V.

22. The apparatus of claim 18, wherein each of the pulses of said electrical stimulation of a target nerve are delivered in monophasic or biphasic modes of a burst pulse train, tonic currents, sinusoidal, and/or combinations of modes.

23. The apparatus of claim 18, wherein the pulses of said electrical stimulation have a frequency from 1 Hz to 100 Hz.

24. The apparatus of claim 18, wherein distributed currents in the electrodes of the SSEA direct a generated electric field to the desired nerve, or nerve area, in a stereotactic method using anatomical parameters.

25. The apparatus of claim 24, wherein said anatomical parameters used in the distribution of the electric currents vary according to the information of the anatomic location of the target nerve.

26. The apparatus of claim 25, wherein said feedback signals are utilized to direct the parameters in the device as closed-loop feedback.

27. The apparatus of claim 26:
wherein said feedback signals used in the closed-loop feedback include nerve conduction velocity (NCV) signals, electromyography (EMG) signals, heart rate, and blood pressure as neurophysiological responses; and
wherein said feedback signals are used to adjust generated stimulation signals from said SSEA, which allows said SASE to monitor autonomic response following the application of stimulation through said SSEA.
